(19) United States
(12) Patent Application Publication
(10) Pub. No.: US 2024/0132522 A1
(43) Pub. Date: Apr. 25, 2024

(54) HETEROCYCLIC COMPOUNDS AND METHODS OF USE THEREOF

(71) Applicant: 1cBio, Inc., Moraga, CA (US)

(72) Inventors: Jayakanth KANKANALA, Frisco, TX (US); Jeremy D. PETTIGREW, Burnaby (CA); Aurelia CONTE, Basel (CH); Jiyun CHEN, Moraga, CA (US); Son Minh PHAM, San Francisco, CA (US)

(21) Appl. No.: 18/458,039

(22) Filed: Aug. 29, 2023

Related U.S. Application Data
(60) Provisional application No. 63/402,417, filed on Aug. 30, 2022.

(51) Int. Cl. C07D 519/00; A61P 35/00; C07D 471/04

(52) U.S. Cl. CPC C07D 519/00; A61P 35/00; C07D 471/04

(57) ABSTRACT

The present disclosure relates to compounds of Formula (I):

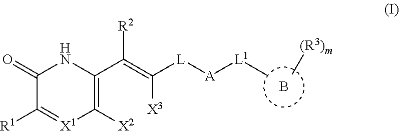

and to their prodrugs, pharmaceutically acceptable salts, pharmaceutical compositions, methods of use, and methods for their preparation. The compounds disclosed herein are useful for modulating PARP1 activity and may be used in the treatment of disorders in which PARP1 activity is implicated, such as cancer.

### CROSS REFERENCE TO RELATED APPLICATIONS

This application claims priority to and the benefit of U.S. Provisional Patent Application No. 63/402,417, filed Aug. 30, 2022, the contents of which are incorporated by reference herein in their entirety for all purposes.

### BACKGROUND

The poly(ADP-ribose) polymerase (PARP) family of enzymes comprises 17 members, among which PARP1 plays an important role in the repair of damaged DNA. PARP1 works through binding to single strand breaks (SSBs) and catalyzing the formation of large branched chains of poly(ADP-ribose) (PAR) in an NAD+ dependent manner. This process, known as PARylation, recruits different DNA repair enzymes and facilitates the release of PARP from DNA. All PARP inhibitors approved thus far are NAD+ competitive inhibitors that compete with NAD+ for the PARP catalytic site. They are thought to work by blocking the enzymatic activity of PARP as well as preventing the release of PARP from damaged DNA, an effect known as PARP trapping. Inhibition of PARP leads to the accumulation of DNA SSBs and double strand breaks (DSBs) and an increase in apoptosis.

There are currently four PARP inhibitors marketed in the US, approved for the treatment of BRCA-mutated breast and ovarian cancer. The inhibitors differ from each other in their PARP trapping efficiency. Homologous recombination (HR)-deficient tumor cells with deleterious alterations in BRCA1 or BRCA2 are particularly sensitive to PARP inhibitors.

The approved PARP inhibitors inhibit both PARP1 and PARP2 enzymes. All these first generation PARP1/2 inhibitors have hematological toxicities like neutropenia, anemia, and thrombocytopenia associated with them. These toxicities are believed to be mediated in large part by PARP2 inhibition.

PARP inhibitors are under testing in combination with immunotherapeutic, chemotherapeutic, and different targeted agents. The various toxicities associated with these agents may be compounded when used in combination with a PARP1/2 inhibitor, so a PARP inhibitor that causes minimal additional toxicity is desirable. Therefore, a highly selective PARP1 inhibitor may have utility not only as a monotherapy for certain cancers, but also as part of combination therapies, and would fill an unmet need for effective, more tolerable treatments for patients suffering from cancer.

### SUMMARY

The present disclosure relates to small molecule, potent inhibitors of poly [ADP-ribose]polymerase 1 (PARP1), designed for the treatment of cancer. It is hypothesized that PARP1-selective inhibitors will have an improved therapeutic index and reduced toxicity as compared to first generation PARP inhibitors.

In some aspects, the present disclosure provides a compound of Formula (I):

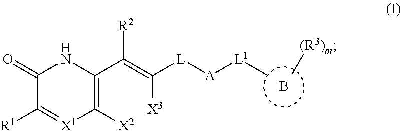

or a pharmaceutically acceptable salt thereof, wherein A, B, L, L¹, X¹, X², X³, R¹, R², R³, and m are detailed herein.

In some aspects, the present disclosure provides a compound obtainable by, or obtained by, a method for preparing a compound as described herein (e.g., a method comprising one or more steps described in Schemes 1-8).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-4).

In some aspects, the present disclosure provides a method of modulating PARP1 activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating PARP1 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating PARP1 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

In an aspect, the present disclosure provides a compound of Formula (I):

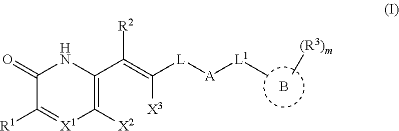

wherein:
A is an optionally substituted C₂₋₉ heterocyclyl comprising at least one nitrogen, or an optionally substituted C₃₋₈ cycloalkylene;
B is a C₆₋₁₀ aryl optionally substituted with one or more R³, or a C₂₋₉ heteroaryl optionally substituted with one or more R³
L is a bond, —CR⁴R⁵—, —C(O)—, —O—, —S—, —S(O)—, —S(O)₂—, —CR⁴R⁵O—, —OCR⁴R⁵—, —NR⁴—, —C(R⁴R⁵)NR^{4′}—, —C(O)NR^{4′}— or —NR^{4′}C(R⁴R⁵)—;
wherein R⁴, R^{4′} and R⁵ are independently —H, C₁₋₆ alkyl, or C₃₋₆ cycloalkyl, or R⁴ and R⁵ combine with the atom or atoms to which they are attached to form a C₂₋₉ heterocyclyl or C₃₋₈ cycloalkyl;
L¹ is a bond, —C(O)—, —NH—, —CH₂NH—, —O—, —CH₂O—, or —CH₂—; X¹ is N or CR¹;
R¹ is —H, C₁₋₆ alkyl, C₃₋₆ cycloalkyl, C₁₋₆ haloalkyl, or C₁₋₆ alkoxy;
X² and X³ are each independently N or CR² provided that both X² and X³ are not both N; R² is —H, halogen, C₁₋₆ alkyl, C₃₋₆ cycloalkyl, C₁₋₆ haloalkyl, or C₁₋₆ alkoxy; R³ is halogen, C₁₋₆ alkyl, C₁₋₆ haloalkyl, C₁₋₆ alkoxy, —C(O)NR⁴R⁵, or —NR⁴C(O)R⁵; and
m is 1, 2, 3, or 4;
with the proviso that when (i) X¹ is N, X² is CH, and X³ is CH, or (ii) when X¹ is CH, X² is N, and X³ is CH, then A is not unsubstituted 1,4-piperazine; and with the proviso that when (i) A is a bridged, bicyclic piperazine, then R³ is not chloro;
or a pharmaceutically acceptable salt thereof.

In some embodiments, the compound is of Formula (II):

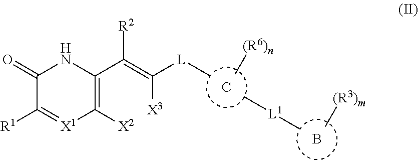

wherein C is a 4-8 membered heterocyclyl comprising at least one nitrogen atom;
R⁶ is a halogen, cyano, —OH, C₁₋₃ alkyl, or two R⁶ together form a carbonyl, C₃₋₆ spirocycl, C₄₋₆ spiroheterocyclyl, C₃₋₆ fused bicyclyl, or C₃₋₆ fused heterocyclyl;
L¹ is bond, —C(O)—, —O—, —CH₂O—, —NR⁷—, or —CH₂NR⁷—;
   wherein R⁷=H, C₁₋₃ alkyl, or cyclopropyl and;
n=0, 1, 2, 3, or 4;
or a pharmaceutically acceptable salt thereof.

In some embodiments, C is a spiro heterocyclyl, a fused heterocyclyl, or a bridged heterocyclyl. In some embodiments, C is a monocyclic heterocyclyl.

In some embodiments, the compound is of Formula (II-a):

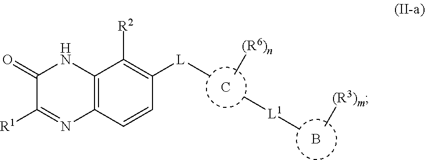

or a pharmaceutically acceptable salt thereof.

In some embodiments, the compound is of Formula (II-b):

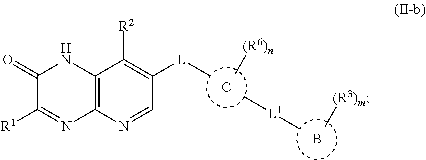

or a pharmaceutically acceptable salt thereof.

In some embodiments, the compound is of Formula (II-c):

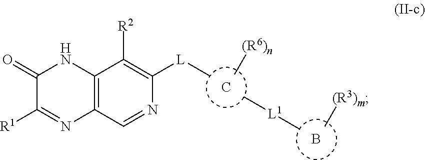

or a pharmaceutically acceptable salt thereof.

In some embodiments, the compound is of Formula (II-d):

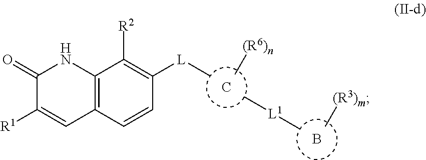

or a pharmaceutically acceptable salt thereof.

In some embodiments, the compound is of Formula (II-e):

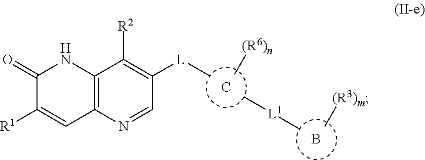

or a pharmaceutically acceptable salt thereof.

In some embodiments, the compound is of Formula (II-f):

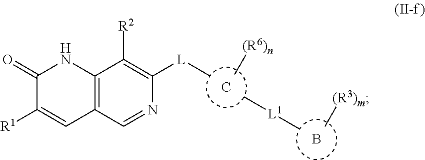

or a pharmaceutically acceptable salt thereof.

In some embodiments, the compound is of Formula (III):

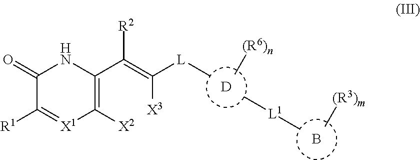

wherein D is a 3-8 membered carbocyclyl or bridged carbobicyclyl; and
R⁷ is H, or C₁₋₆ alkyl;
or a pharmaceutically acceptable salt thereof.

In some embodiments, the compound is of Formula (III-a):

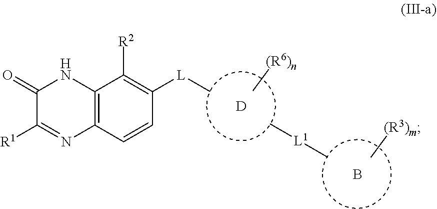

or a pharmaceutically acceptable salt thereof.

In some embodiments, the compound is of Formula (III-b):

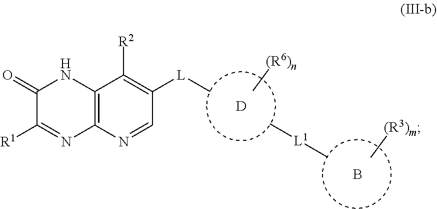

or a pharmaceutically acceptable salt thereof.

In some embodiments, the compound is of Formula (III-c):

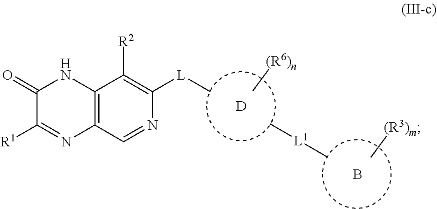

or a pharmaceutically acceptable salt thereof.

In some embodiments, the compound is of Formula (III-d):

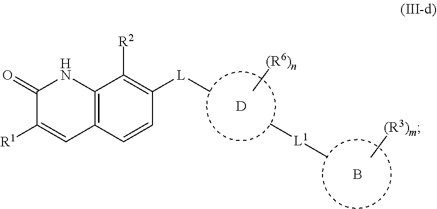

or a pharmaceutically acceptable salt thereof.

In some embodiments, the compound is of Formula (III-e):

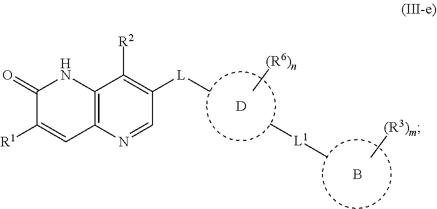

or a pharmaceutically acceptable salt thereof.

In some embodiments, the compound is of Formula (III-f):

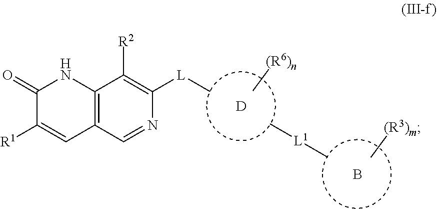

or a pharmaceutically acceptable salt thereof.

In some embodiments, L is —CR⁴R⁵—NR^{4′}— or —C(O)NR^{4′}—.

In some embodiments:
R¹ is ethyl, X¹ is CH, X² is N, X³ is CH, and R² is H;
R¹ is methyl, X¹ is N, X² is CH, X³ is CH, and R² is fluoro;
R¹ is ethyl, X¹ is N, X² is N, X³ is CH, and R² is H;
R¹ is ethyl, X¹ is N, X² is CH, X³ is N, and R² is H;
R¹ is ethyl, X¹ is CH, X² is CH, X³ is CH, and R² is H;
R¹ is ethyl, X¹ is CH, X² is CH, X³ is N, and R² is H;
R¹ is methyl, X¹ is N, X² is CH, X³ is CH, and R² is H;
R¹ is trifluoromethyl, X¹ is N, X² is CH, X³ is CH, and R² is H; or
R¹ is 1,1,1-trifluoroethyl, X¹ is N, X² is CH, X³ is CH, and R² is H.

In some embodiments, B is a C₂₋₉ heteroaryl optionally substituted with one or more R³. In some embodiments, B is selected from

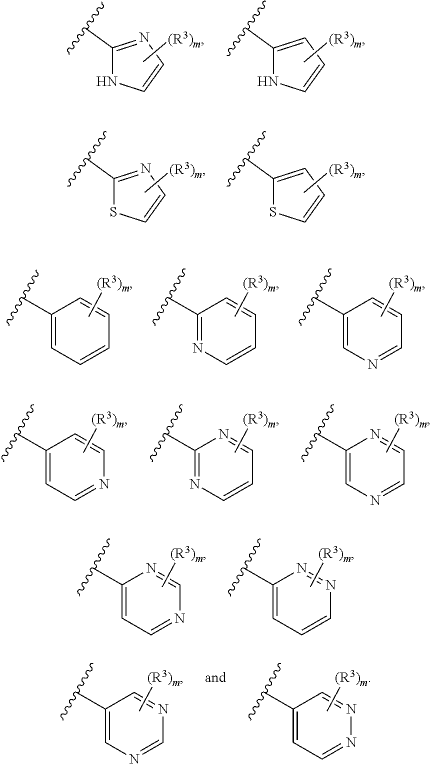

In some embodiments, B is

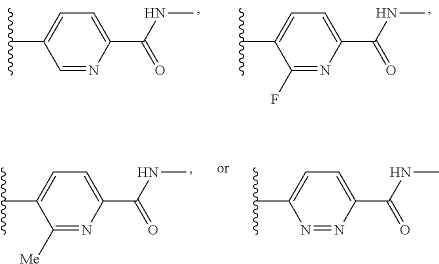

wherein [symbol] denotes points of attachment. In some embodiments, wherein B is a C₆₋₁₀ aryl optionally substituted with one or more R³. In some embodiments, B is

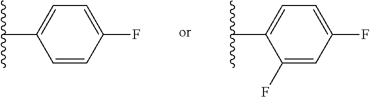

wherein [symbol] denotes points of attachment.

In some embodiments, C is

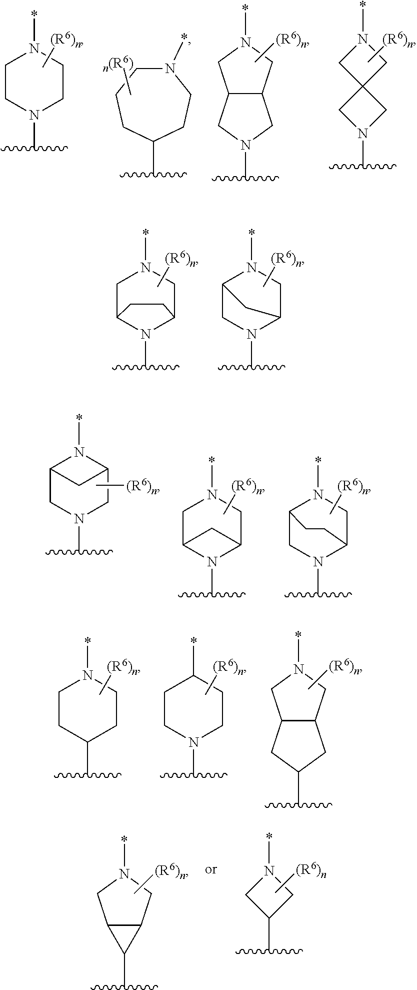

wherein * and [symbol] denote points of attachment. In some embodiments, C is

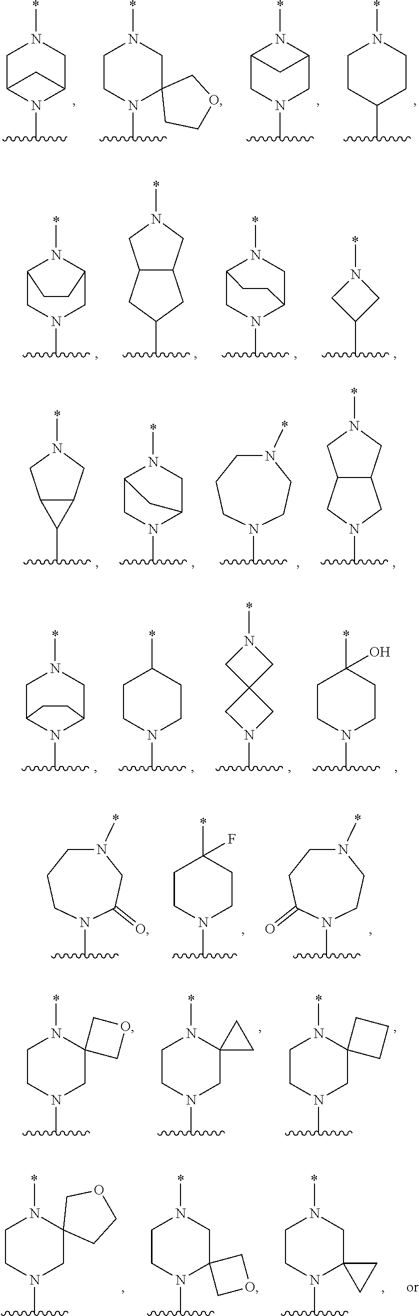

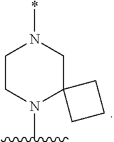 [SMILES: *N1CCN(*)C2(CCC2)C1]

In some embodiments, D is or

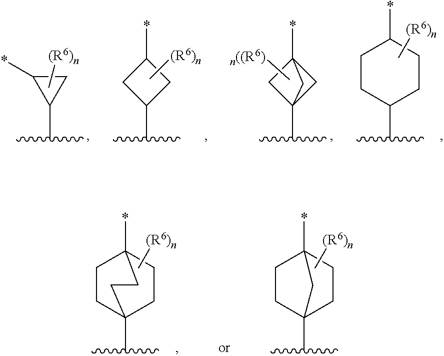

wherein * and [symbol] denote points of attachment. In some embodiments, D is

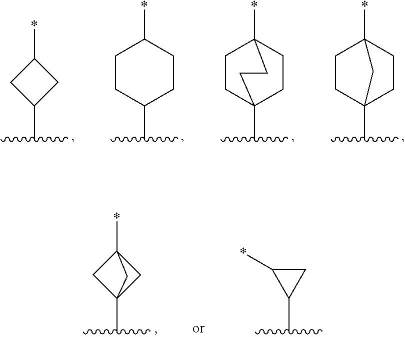 [SMILES: *C1CC(*)C1 ; *C1CCC(*)CC1 ; *C12CCC(*)(CC1)CC2 ; *C12CCC(*)(CC1)C2 ; *C12CC(*)(C1)C2 ; *C1CC1*]

wherein * and [symbol] denote points of attachment.

In some embodiments, L is

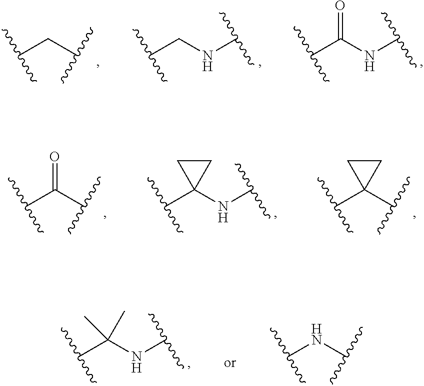

wherein [symbol] denotes points of attachment.

In some embodiments, L¹ is a bond

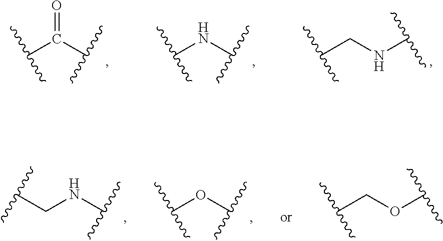

wherein [symbol] denotes points of attachment.

In some embodiments, the present disclosure provides a compound having any one of the structures shown in Table 1, or a pharmaceutically acceptable salt thereof.

In some embodiments, the present disclosure provides a pharmaceutical composition comprising any compound disclosed herein and a pharmaceutically acceptable excipient.

In some embodiments, the present disclosure is directed to a method of treating a cancer with a compound of the present disclosure or a pharmaceutical composition comprising a compound of the present disclosure to a subject in need thereof.

In some embodiments, the present disclosure is directed to a method of treating a cancer with a BRCAness phenotype comprising administering a compound of the present disclosure or a pharmaceutical composition thereof to a subject in need thereof.

In some embodiments, the present disclosure is directed to a method of treating a breast cancer, ovarian cancer, prostate cancer, or pancreatic cancer comprising administering a compound of the present disclosure to a subject in need thereof.

In some embodiments, the present disclosure provides a method of treating a HR-deficient cancer comprising administering a compound of the present disclosure to a subject in need thereof.

In some embodiments, the present disclosure provides a method of treating a BRCA1- or BRCA2-mutant cancer, comprising administering an effective amount of a compound of the present disclosure, or the pharmaceutical composition thereof, to a subject in need thereof.

In some embodiments, the present disclosure provides a method of treating a HR-deficient cancers, a BRCA1- or BRCA2-mutant cancer, or a cancer with a BRCAness phenotype in a subject, by administering an effective amount of any one of the compounds or pharmaceutical compositions disclosed herein to a subject in need thereof.

In some embodiments, the cancer with a BRCAness phenotype is selected from the group consisting of breast cancer, ovarian cancer, prostate cancer, and pancreatic cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to heterocyclic derivatives, prodrugs, and pharmaceutically acceptable salts thereof, which may modulate PARP1 activity and are accordingly useful in methods of treatment of the human or animal body. The present disclosure also relates to processes for the preparation of these compounds, to pharmaceutical compositions comprising them and to their use in the treatment of disorders in which PARP1 is implicated.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables X, L, and Y are described herein, the disclosure may be interpreted as excluding structures for non-operable compound caused by certain combinations of variables X, L, and Y (e.g., when each of X, L, and Y is —O—).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups.

In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

It is understood that when a variable has two attachments to the rest of the formula of the compound, the two attachments could be at the same atom or different atoms of the variable. For example, when a variable (e.g., variable X) is cycloalkyl or heterocycloalkyl, and has two attachments to the rest of the formula of the compound, the two attachments could be at the same atom or different atoms of the cycloalkyl or heterocycloalkyl.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. For example, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein.

Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight (w/w). Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As used herein, the phrase “compound of the disclosure” refers to those compounds which are disclosed herein, both generically and specifically.

### Compounds of the Present Disclosure

In some aspects, the present disclosure provides a compound of Formula (I):

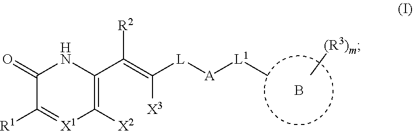

or a pharmaceutically acceptable salt thereof, wherein:
A is an optionally substituted C₂₋₉ heterocyclyl comprising at least one nitrogen, or an optionally substituted C₃₋₈ cycloalkylene;
B is a C₆₋₁₀ aryl optionally substituted with one or more R³, or a C₂₋₉ heteroaryl optionally substituted with one or more R³;
L is a bond, —CR⁴R⁵—, —C(O)—, —O—, —S—, —S(O)—, —S(O)₂—, —CR⁴R⁵O—, —OCR⁴R⁵—, —NR⁴—, —C(R⁴R⁵)NR^{4′}—, —C(O)NR^{4′}— or —NR^{4′}C(R⁴R⁵)—;
wherein R⁴, R^{4′} and R⁵ are independently —H, C₁₋₆ alkyl, or C₃₋₆ cycloalkyl, or R⁴ and R⁵ combine with the atom or atoms to which they are attached to form a C₂₋₉ heterocyclyl or C₃₋₈ cycloalkyl;
L¹ is a bond, —C(O)—, —NH—, —CH₂NH—, —O—, —CH₂O—, or —CH₂—;
X¹ is N or CR¹;
R¹ is H, C₁₋₆ alkyl, C₃₋₆ cycloalkyl, C₁₋₆ haloalkyl, or C₁₋₆ alkoxy;
X² and X³ are each independently N or CR² provided that X² and X³ are not both N;
R² is —H, halogen, C₁₋₆ alkyl, C₃₋₆ cycloalkyl, C₁₋₆ haloalkyl, or C₁₋₆ alkoxy;
R³ is halogen, C₁₋₆ alkyl, C₁₋₆ haloalkyl, C₁₋₆ alkoxy, —C(O)NR⁴R⁵, or —NR⁴C(O)R⁵; and
m is 1, 2, 3, or 4;
with the proviso that when (i) X¹ is N, X² is CH, and X³ is CH, or (ii) when X¹ is CH, X² is N, and X³ is CH, then A is not unsubstituted 1,4-piperazine; and with the proviso that when (i) A is a bridged, bicyclic piperazine, then R³ is not chloro.

In some aspects, the present disclosure provides a compound of Formula (II):

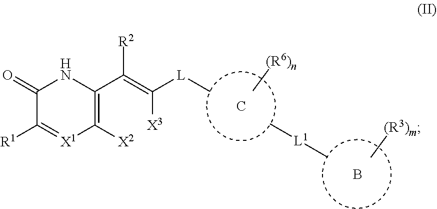

or a pharmaceutically acceptable salt thereof, wherein B, L, X¹, X², X³, R¹, R², R³, R⁴, R^{4′}, R⁵, and m are described herein for Formula (I) and:
C is a 4-8 membered heterocyclyl comprising at least one nitrogen atom;
R⁶ is a halogen, cyano, —OH, C₁₋₃ alkyl, or two R⁶ together form a carbonyl, C₃₋₆ spirocycl, C₄₋₆ spiroheterocyclyl, C₃₋₆ fused bicyclyl, or C₃₋₆ fused heterocyclyl;
L¹ is a bond, —C(O)—, —O—, —CH₂O—, —NR⁷—, or —CH₂NR⁷—;
wherein R⁷=—H, C₁₋₃ alkyl, or cyclopropyl; and
n=0, 1, 2, 3, or 4.

In some aspects, the present disclosure provides a compound of Formula (III):

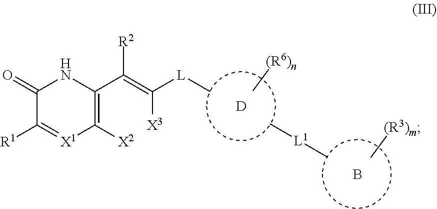

or a pharmaceutically acceptable salt thereof, wherein B, L, L¹, X¹, X², X³, R¹, R², R³, R⁴, R^{4′}, R⁵, R⁶, m, and n are described herein for formula (II) and:
D is a 3-8 membered carbocyclyl or bridged carbobicyclyl; and
R⁷ is —H, or C₁₋₆ alkyl.

It is understood that, for a compound of the present disclosure, variables A, B, C, D, L, L¹, X¹, X², X³, R¹, R², R³, R⁴, R^{4′}, R⁵, R⁶, R⁷, m, and n can each be, where applicable, selected from the groups described herein, and any group described herein for any of variables A, B, C, D, L, L¹, X¹, X², X³, R¹, R², R³, R⁴, R^{4′}, R⁵, R⁶, R⁷, m, and n can be combined, where applicable, with any group described herein for one or more of the remainder of variables A, B, C, D, L, L¹, X¹, X², X³, R¹, R², R³, R⁴, R^{4′}, R⁵, R⁶, R⁷, m, and n.

In some embodiments, Formula (II) is Formulae (II-a)-(II-f) or a pharmaceutically acceptable salt thereof.

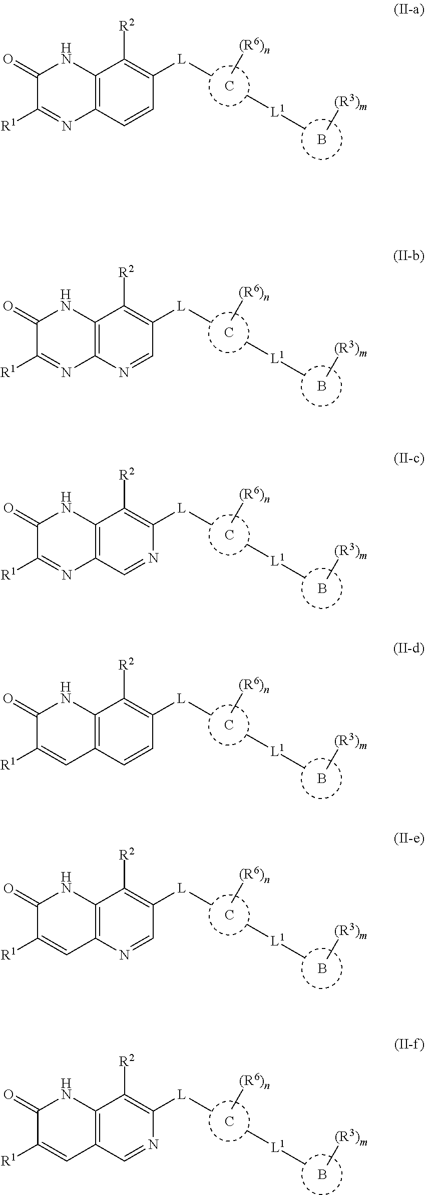

In some embodiments, Formula (III) is Formulae (III-a)-(III-f) or a pharmaceutically acceptable salt thereof.

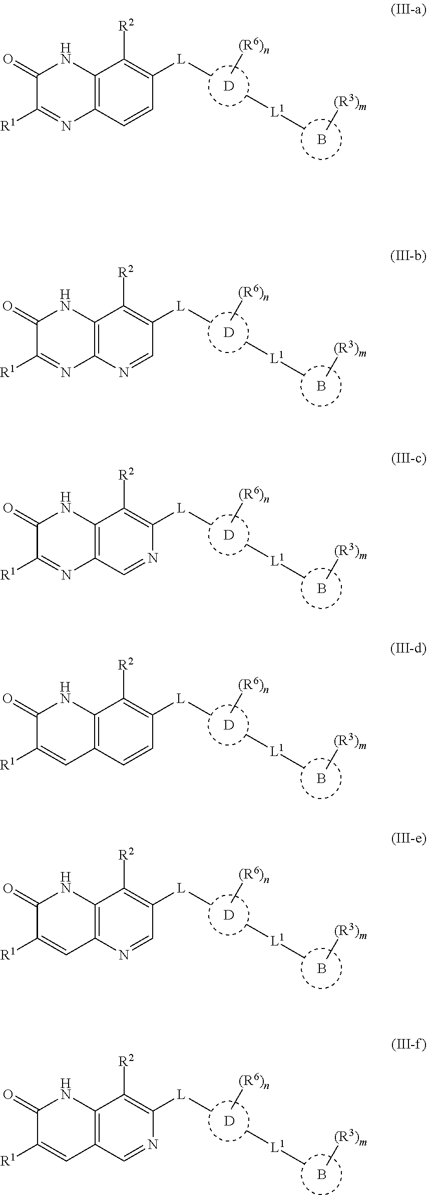

In some embodiments, A is an optionally substituted C₂₋₉ heterocyclyl comprising at least one nitrogen, or an optionally substituted C₃₋₈ cycloalkylene. In some embodiments, A

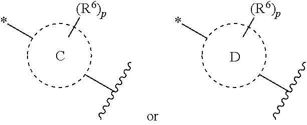

wherein * and [symbol] denote points of attachment. In some embodiments, A is

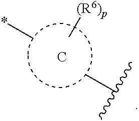

In some embodiments, A is

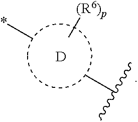

In some embodiments, B is a C₆₋₁₀ aryl optionally substituted with one or more R³, or a C₂₋₉ heteroaryl optionally substituted with one or more R³. In some embodiments, B is

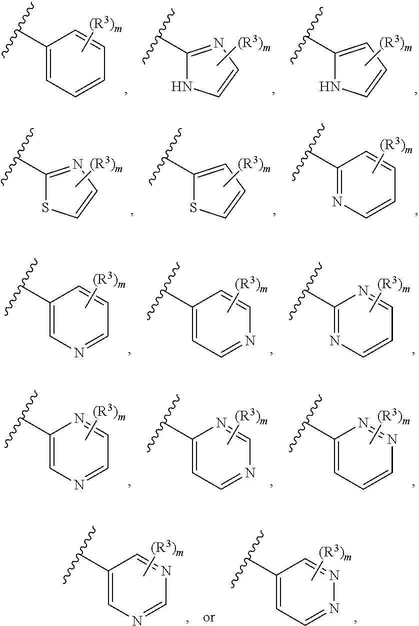

wherein [symbol] denotes a point of attachment. In some embodiments, B is

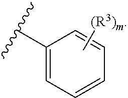

In some embodiments, B is

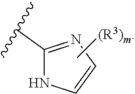

In some embodiments, B is

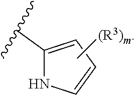

In some embodiments, B is

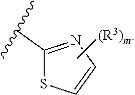

In some embodiments, B is

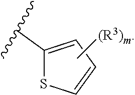

In some embodiments, B is

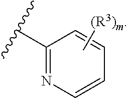

In some embodiments, B is

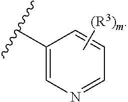

In some embodiments, B is

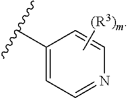

In some embodiments, B is

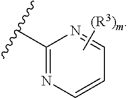

In some embodiments, B is

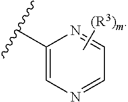

In some embodiments, B is

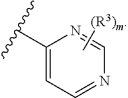

In some embodiments, B is

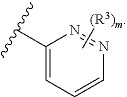

In some embodiments, B is

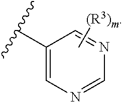

In some embodiments, B is

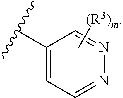

In some embodiments, C is a 4-8 membered heterocyclyl comprising at least one nitrogen atom. In some embodiments, C is

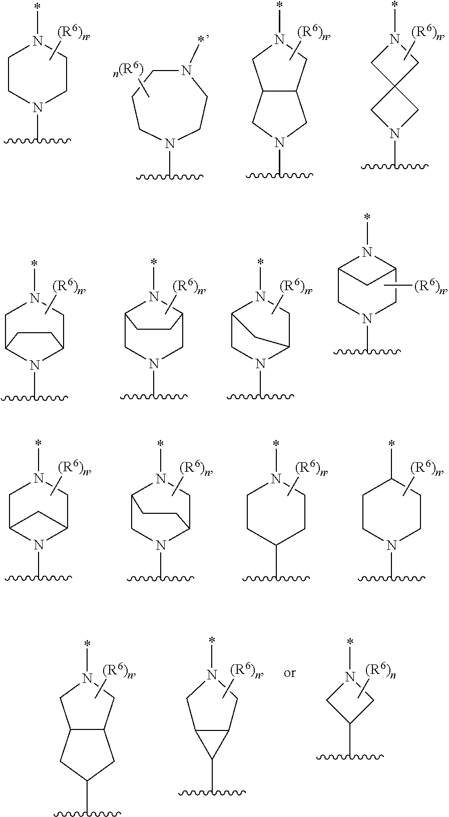

wherein * and [symbol] denote points of attachment. In some embodiments, C is

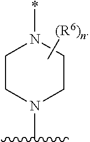

In some embodiments, C is

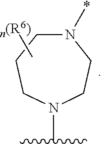

In some embodiments, C is

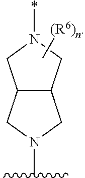

In some embodiments, C is

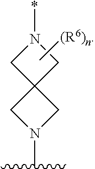

In some embodiments, C is

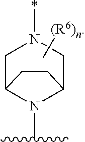

In some embodiments, C is

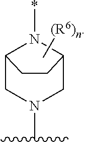

In some embodiments, C is

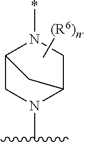

In some embodiments, C is

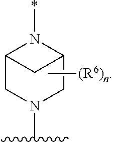

In some embodiments, C is

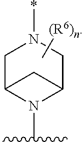

In some embodiments, C is

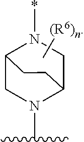

In some embodiments, C is

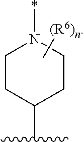

In some embodiments, C is

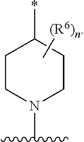

In some embodiments, C is

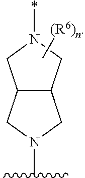

In some embodiments, C is

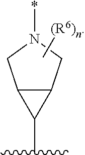

In some embodiments, C is

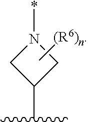

In some embodiments, D is a 3-8 membered carbocyclyl or bridged carbobicyclyl. In some embodiments, D is

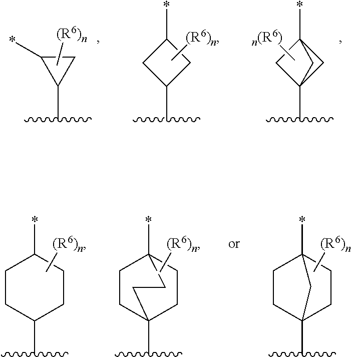

wherein * and [symbol] denote points of attachment. In some embodiments, D is

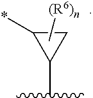

In some embodiments, D is

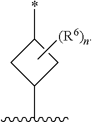

In some embodiments, D is

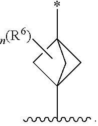

In some embodiments, D is

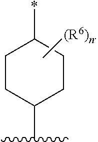

In some embodiments, D is

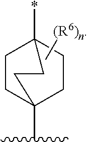

In some embodiments, D is

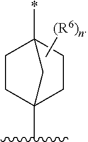

In some embodiments, L is a bond, —CR⁴R⁵—, —C(O)—, —O—, —S—, —S(O)—, —S(O)₂—, —CR⁴R⁵O—, —OCR⁴R⁵—, —NR⁴—, —C(R⁴R⁵)NR^{4′}—, —C(O)NR^{4′}— or —NR^{4′}C(R⁴R⁵)—. In some embodiments, L is a bond. In some embodiments, L is —CR⁴R⁵—. In some embodiments, L is —C(O)—. In some embodiments, L is —O—. In some embodiments, L is —S—. In some embodiments, L is —S(O)—. In some embodiments, L is —S(O)₂—. In some embodiments, L is —CR⁴R⁵O—. In some embodiments, L is —OCR⁴R⁵—. In some embodiments, L is —NR⁴—. In some embodiments, L is —C(R⁴R⁵)NR^{4′}—. In some embodiments, L is —C(O)NR^{4′}—. In some embodiments, L is —NR^{4′}C(R⁴R⁵)—.

In some embodiments, L¹ is a bond, —C(O)—, —NH—, —CH₂NH—, —O—, —CH₂O—, or —CH₂—. In some embodiments, L¹ is a bond. In some embodiments, L¹ is —C(O)—. In some embodiments, L¹ is a —NH—. In some embodiments, L¹ is a —CH₂NH—. In some embodiments, L¹ is —O—. In some embodiments, L¹ is —CH₂O—. In some embodiments, L¹ is —CH₂—.

In some embodiments, X¹ is N or CR¹. In some embodiments, X¹ is N. In some embodiments, X¹ is CR¹.

In some embodiments, X² and X³ are each independently N or CR² provided that both X² and X³ are not N. In some embodiments, both X² and X³ are CR². In some embodiments, X² is N and X³ is CR². In some embodiments, X³ is N and X² is CR².

In some embodiments, R¹ is —H, C₁₋₆ alkyl, C₃₋₆ cycloalkyl, C₁₋₆ haloalkyl, or C₁₋₆ alkoxy. In some embodiments, R¹ is —H, -Me, -Et, —Pr, -iPr, cyclopropyl, —CF₃, —CHFMe, —CF₂Me, —CH₂CH₂F, —CH₂CHF₂, —CH₂CF₃, or —OMe. In some embodiments, R¹ is —H. In some embodiments, R¹ is -Me. In some embodiments, R¹ is -Et. In some embodiments, R¹ is —Pr. In some embodiments, R¹ is -iPr. In some embodiments, R¹ is cyclopropyl. In some embodiments, R¹ is —CF₃. In some embodiments, R¹ is —CHFMe. In some embodiments, R¹ is —CF₂Me. In some embodiments, R¹ is —CH₂CH₂F. In some embodiments, R¹ is —CH₂CHF₂. In some embodiments, R¹ is —CH₂CF₃. In some embodiments, R¹ is —OMe.

In some embodiments, R² is —H, halogen, C₁₋₆ alkyl, C₃₋₆ cycloalkyl, C₁₋₆ haloalkyl, or C₁₋₆ alkoxy. In some embodiments, R² is —H, —F, —Cl, —Br, -Me, -Et, -iPr, cyclopropyl, —CF₃, or —OMe. In some embodiments, R² is —H. In some embodiments, R² is —F. In some embodiments, R² is —Cl. In some embodiments, R² is —Br. In some embodiments, R² is -Me. In some embodiments, R² is -Et. In some embodiments, R² is -iPr. In some embodiments, R² is cyclopropyl. In some embodiments, R² is —CF₃. In some embodiments, R² is —OMe.

In some embodiments, R³ is halogen, C₁₋₆ alkyl, C₁₋₆ haloalkyl, C₁₋₆ alkoxy, —C(O)NR⁴R⁵, or —NR⁴C(O)R⁵. In some embodiments, R³ is —F, —Cl, —Br, -Me, —CF₃, —OMe, —C(O)NR⁴R⁵, or —NR⁴C(O)R⁵. In some embodiments, R³ is —F. In some embodiments, R³ is —Cl. In some embodiments, R³ is —Br. In some embodiments, R³ is -Me. In some embodiments, R³ is —CF₃. In some embodiments, R³ is —OMe. In some embodiments, R³ is —C(O)NR⁴R⁵. In some embodiments, R³ is —NR⁴C(O)R⁵.

In some embodiments, R⁴ and R⁵ are independently —H, C₁₋₆ alkyl, or C₃₋₆ cycloalkyl, or R⁴ and R⁵ combine with the atom or atoms to which they are attached to form a C₂₋₉ heterocyclyl or C₃₋₈ cycloalkyl. In some embodiments, R⁴ and R⁵ are independently —H, -Me, -Et, cyclopropyl or R⁴ and R⁵ together form the following rings,

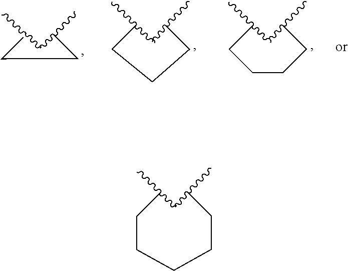

wherein [symbol] denotes points of attachment. In some embodiments, R⁴ and R⁵ are both —H. In some embodiments, R⁴ is —H and R⁵ is -Me. In some embodiments, R⁴ is —H and R⁵ is -Et. In some embodiments, R⁴ is —H and R⁵ is cyclopropyl. In some embodiments, R⁴ and R⁵ are both -Me. In some embodiments, R⁴ and R⁵ together form the following ring,

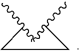

In some embodiments, R⁴ and R⁵ together form the following ring,

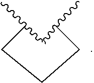

In some embodiments, R⁴ and R⁵ together form the following ring,

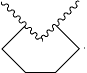

In some embodiments, R⁴ and R⁵ together form the following ring,

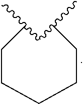

In some embodiments, R^{4′} is —H, C₁₋₆ alkyl, or C₃₋₆ cycloalkyl. In some embodiments, R^{4′} is —H, -Me, -Et, cyclopropyl. In some embodiments, R^{4′} is —H. In some embodiments, R^{4′} is -Me. In some embodiments, R^{4′} is -Et. In some embodiments, R^{4′} is cyclopropyl.

In some embodiments, R⁶ is a halogen, cyano, —OH, C₁₋₃ alkyl, or two R⁶'s together form a carbonyl, C₃₋₆ spirocycl, C₄₋₆ spiroheterocyclyl, C₃₋₆ fused bicyclyl, or C₃₋₆ fused heterocyclyl. In some embodiments, R⁶ is a —F, cyano, —OH, -Me, -Et, —Pr, -iPr or two R⁶'s together form the following rings,

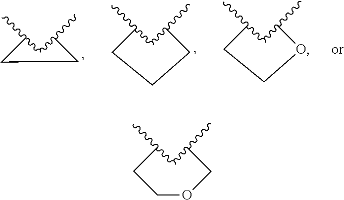

wherein [symbol] denotes points of attachment. In some embodiments, R⁶ is —F. In some embodiments, R⁶ is cyano. In some embodiments, R⁶ is —OH. In some embodiments, R⁶ is -Me. In some embodiments, R⁶ is -Et. In some embodiments, R⁶ is —Pr. In some embodiments, R⁶ is -iPr. In some embodiments, two R⁶'s together form

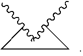

In some embodiments, two R⁶'s together form

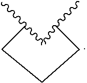

In some embodiments, two R⁶'s together form

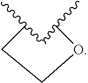

In some embodiments, two R⁶'s together form

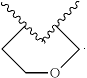

In some embodiments, R⁷ is —H, cyclopropyl, or C₁₋₆ alkyl. In some embodiments, R⁷ is —H, cyclopropyl, -Me, -Et, —Pr, or -iPr. In some embodiments, R⁷ is —H. In some embodiments, R⁷ is cyclopropyl. In some embodiments, R⁷ is -Me. In some embodiments, R⁷ is -Et. In some embodiments, R⁷ is —Pr. In some embodiments, R⁷ is -iPr.

In some embodiments, m is 1, 2, 3, or 4. In some embodiments, m is 1. In some embodiments, m is 2. In some embodiments, m is 3. In some embodiments, m is 4.

In some embodiments, n is 0, 1, 2, 3, or 4. In some embodiments, n is 0. In some embodiments, n is 1. In some embodiments, n is 2. In some embodiments, n is 3. In some embodiments, n is 4.

In some embodiments, the compound is selected from the compounds described in Table 1 and prodrugs and pharmaceutically acceptable salts thereof.

TABLE 1

| Compound | | |
|---|---|---|
| No. | Name | Structure |
| 1 | 5-(6-((7-ethyl-6-oxo-5,6-dihydro-1,5- naphthyridin-3-yl)methyl)-3,6- diazabicyclo[3.1.1]heptan-3-yl)-N- methylpicolinamide | 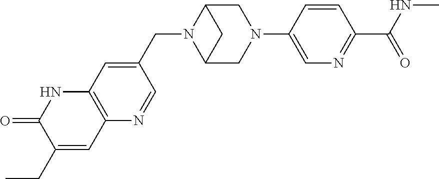 [SMILES: CCc1cc2ncc(CN3C4CC3CN(c3ccc(C(=O)NC)nc3)C4)cc2[nH]c1=O] |
| 2 | 5-(3-((7-ethyl-6-oxo-5,6-dihydro-1,5- naphthyridin-3-yl)methyl)-3,6- diazabicyclo[3.1.1]heptan-6-yl)-N- methylpicolinamide | 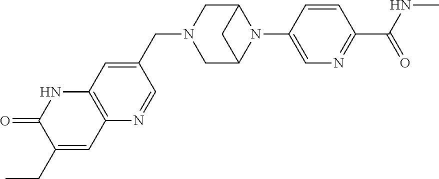 [SMILES: CCc1cc2ncc(CN3CC4CC(C3)N4c3ccc(C(=O)NC)nc3)cc2[nH]c1=O] |
| 3 | 5-(8-((7-ethyl-6-oxo-5,6-dihydro-1,5- naphthyridin-3-yl)methyl)-3,8- diazabicyclo[3.2.1]octan-3-yl)-N- methylpicolinamide | 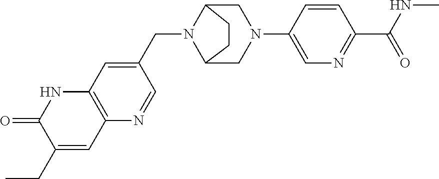 [SMILES: CCc1cc2ncc(CN3C4CCC3CN(c3ccc(C(=O)NC)nc3)C4)cc2[nH]c1=O] |
| 4 | 5-(5-((7-ethyl-6-oxo-5,6-dihydro-1,5- naphthyridin-3-yl)methyl)-2,5- diazabicyclo[2.2.2]octan-2-yl)-N- methylpicolinamide | 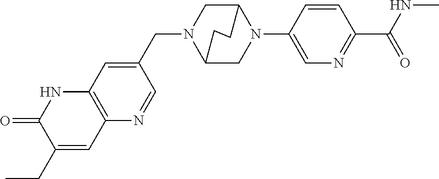 [SMILES: CCc1cc2ncc(CN3CC4CCC3CN4c3ccc(C(=O)NC)nc3)cc2[nH]c1=O] |
| 5 | 5-(5-((7-ethyl-6-oxo-5,6-dihydro-1,5- naphthyridin-3-yl)methyl)-2,5- diazabicyclo[2.2.1]heptan-2-yl)-N- methylpicolinamide | 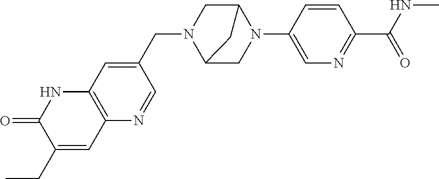 [SMILES: CCc1cc2ncc(CN3CC4CC3CN4c3ccc(C(=O)NC)nc3)cc2[nH]c1=O] |
| 6 | 5-(4-((7-ethyl-6-oxo-5,6-dihydro-1,5- naphthyridin-3-yl)methyl)-1,4-diazepan-1-yl)-N- methylpicolinamide | 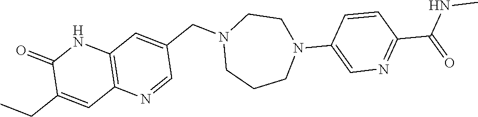 [SMILES: CCc1cc2ncc(CN3CCCN(c4ccc(C(=O)NC)nc4)CC3)cc2[nH]c1=O] |
| 7 | 5-(5-((7-ethyl-6-oxo-5,6-dihydro-1,5- naphthyridin-3-yl)methyl)hexahydropyrrolo[3,4- c]pyrrol-2(1H)-yl)-N-methylpicolinamide | 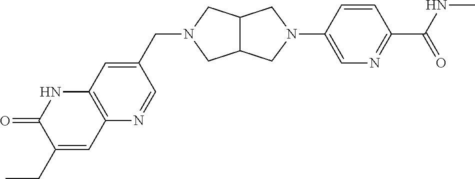 [SMILES: CCc1cc2ncc(CN3CC4CN(c5ccc(C(=O)NC)nc5)CC4C3)cc2[nH]c1=O] |
| 8 | 5-(3-((7-ethyl-6-oxo-5,6-dihydro-1,5- naphthyridin-3-yl)methyl)-3,8- diazabicyclo[3.2.1]octan-8-yl)-N- methylpicolinamide | 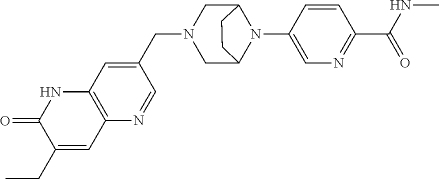 [SMILES: CCc1cc2ncc(CN3CC4CCC(C3)N4c3ccc(C(=O)NC)nc3)cc2[nH]c1=O] |
| 9 | 5-(6-((7-ethyl-6-oxo-5,6-dihydro-1,5- naphthyridin-3-yl)methyl)-2,6- diazaspiro[3.3]heptan-2-yl)-N- methylpicolinamide | 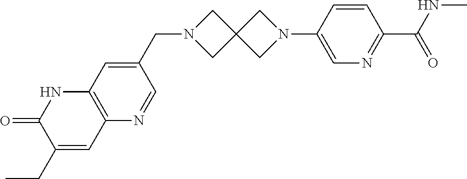 [SMILES: CCc1cc2ncc(CN3CC4(C3)CN(c3ccc(C(=O)NC)nc3)C4)cc2[nH]c1=O] |
| 10 | 5-(4-((7-ethyl-6-oxo-5,6-dihydro-1,5- naphthyridin-3-yl)methyl)-2-oxo-1,4-diazepan- 1-yl)-N-methylpicolinamide | 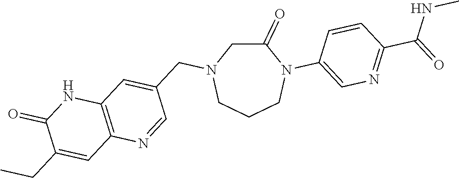 [SMILES: CCc1cc2ncc(CN3CCCN(c4ccc(C(=O)NC)nc4)C(=O)C3)cc2[nH]c1=O] |
| 11 | 5-(4-((7-ethyl-6-oxo-5,6-dihydro-1,5- naphthyridin-3-yl)methyl)-7-oxo-1,4-diazepan- 1-yl)-N-methylpicolinamide | 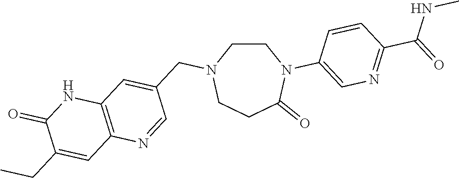 [SMILES: CCc1cc2ncc(CN3CCC(=O)N(c4ccc(C(=O)NC)nc4)CC3)cc2[nH]c1=O] |
| 12 | 5-(5-((7-ethyl-6-oxo-5,6-dihydro-1,5- naphthyridin-3-yl)methyl)-2-oxa-5,8- diazaspiro[3.5]nonan-8-yl)-N- methylpicolinamide | 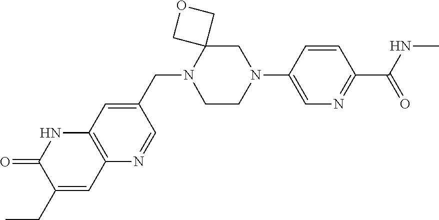 [SMILES: CCc1cc2ncc(CN3CCN(c4ccc(C(=O)NC)nc4)CC34COC4)cc2[nH]c1=O] |
| 13 | 5-(4-((7-ethyl-6-oxo-5,6-dihydro-1,5- naphthyridin-3-yl)methyl)-4,7- diazaspiro[2.5]octan-7-yl)-N- methylpicolinamide | 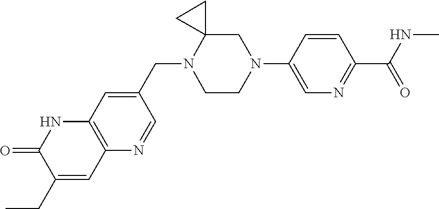 [SMILES: CCc1cc2ncc(CN3CCN(c4ccc(C(=O)NC)nc4)CC34CC4)cc2[nH]c1=O] |
| 14 | 5-(5-((7-ethyl-6-oxo-5,6-dihydro-1,5- naphthyridin-3-yl)methyl)-5,8- diazaspiro[3.5]nonan-8-yl)-N- methylpicolinamide | 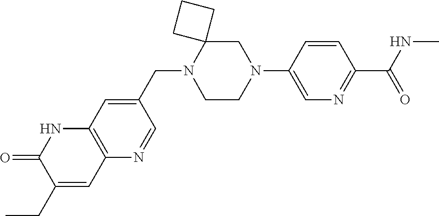 [SMILES: CCc1cc2ncc(CN3CCN(c4ccc(C(=O)NC)nc4)CC34CCC4)cc2[nH]c1=O] |
| 15 | 5-(6-((7-ethyl-6-oxo-5,6-dihydro-1,5- naphthyridin-3-yl)methyl)-2-oxa-6,9- diazaspiro[4.5]decan-9-yl)-N- methylpicolinamide | 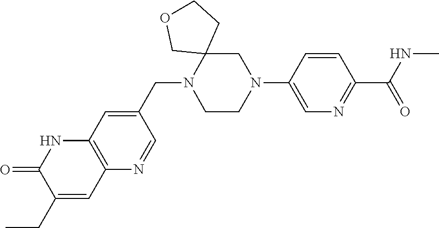 [SMILES: CCc1cc2ncc(CN3CCN(c4ccc(C(=O)NC)nc4)CC34CCOC4)cc2[nH]c1=O] |
| 16 | 5-(8-((7-ethyl-6-oxo-5,6-dihydro-1,5- naphthyridin-3-yl)methyl)-2-oxa-5,8- diazaspiro[3.5]nonan-5-yl)-N- methylpicolinamide | 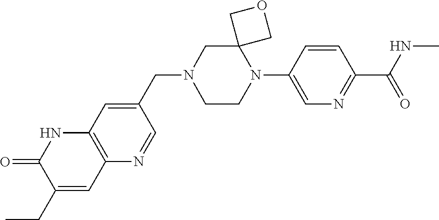 [SMILES: CCc1cc2ncc(CN3CCN(c4ccc(C(=O)NC)nc4)C4(COC4)C3)cc2[nH]c1=O] |
| 17 | 5-(7-((7-ethyl-6-oxo-5,6-dihydro-1,5- naphthyridin-3-yl)methyl)-4,7- diazaspiro[2.5]octan-4-yl)-N- methylpicolinamide | 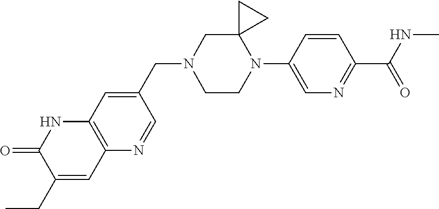 [SMILES: CCc1cc2ncc(CN3CCN(c4ccc(C(=O)NC)nc4)C4(CC4)C3)cc2[nH]c1=O] |
| 18 | 5-(8-((7-ethyl-6-oxo-5,6-dihydro-1,5- naphthyridin-3-yl)methyl)-5,8- diazaspiro[3.5]nonan-5-yl)-N- methylpicolinamide | 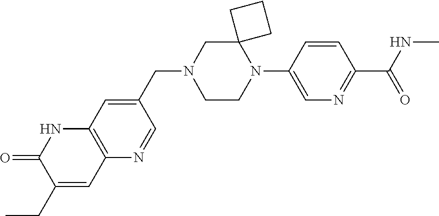 [SMILES: CCc1cc2ncc(CN3CCN(c4ccc(C(=O)NC)nc4)C4(CCC4)C3)cc2[nH]c1=O] |
| 19 | 5-(9-((7-ethyl-6-oxo-5,6-dihydro-1,5- naphthyridin-3-yl)methyl)-2-oxa-6,9- diazaspiro[4.5]decan-6-yl)-N- methylpicolinamide | 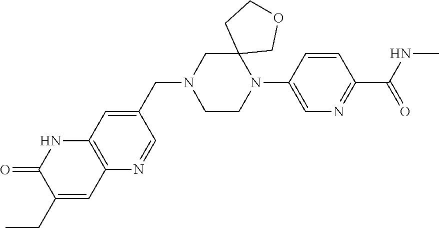 [SMILES: CCc1cc2ncc(CN3CCN(c4ccc(C(=O)NC)nc4)C4(CCOC4)C3)cc2[nH]c1=O] |
| 20 | 5-((1-((7-ethyl-6-oxo-5,6-dihydro-1,5- naphthyridin-3-yl)methyl)piperidin-4-yl)amino)- N-methylpicolinamide | 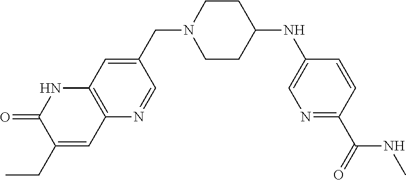 [SMILES: CCc1cc2ncc(CN3CCC(Nc4ccc(C(=O)NC)nc4)CC3)cc2[nH]c1=O] |
| 21 | 5-((2-((7-ethyl-6-oxo-5,6-dihydro-1,5- naphthyridin-3- yl)methyl)octahydrocyclopenta[c]pyrrol-5- yl)amino)-N-methylpicolinamide | 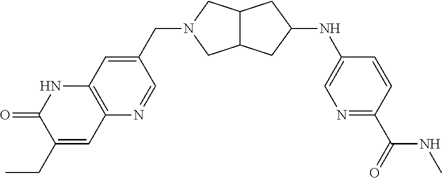 [SMILES: CCc1cc2ncc(CN3CC4CC(Nc5ccc(C(=O)NC)nc5)CC4C3)cc2[nH]c1=O] |
| 22 | 5-((3-((7-ethyl-6-oxo-5,6-dihydro-1,5- naphthyridin-3-yl)methyl)-3- azabicyclo[3.1.0]hexan-6-yl)amino)-N- methylpicolinamide | 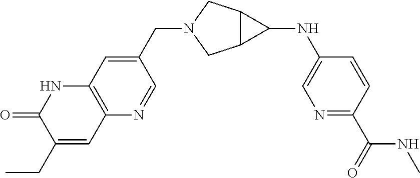 [SMILES: CCc1cc2ncc(CN3CC4C(C3)C4Nc3ccc(C(=O)NC)nc3)cc2[nH]c1=O] |
| 23 | 5-((1-((7-ethyl-6-oxo-5,6-dihydro-1,5- naphthyridin-3-yl)methyl)azetidin-3-yl)amino)- N-methylpicolinamide | 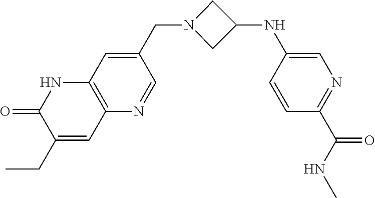 [SMILES: CCc1cc2ncc(CN3CC(Nc4ccc(C(=O)NC)nc4)C3)cc2[nH]c1=O] |
| 24 | 5-(((1-((7-ethyl-6-oxo-5,6-dihydro-1,5- naphthyridin-3-yl)methyl)azetidin-3- yl)methyl)amino)-N-methylpicolinamide | 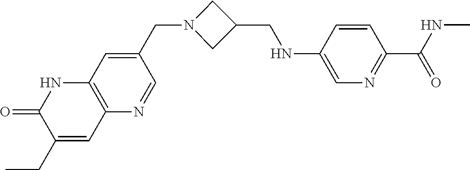 [SMILES: CCc1cc2ncc(CN3CC(CNc4ccc(C(=O)NC)nc4)C3)cc2[nH]c1=O] |
| 25 | 5-((1-((7-ethyl-6-oxo-5,6-dihydro-1,5- naphthyridin-3-yl)methyl)azetidin-3-yl)oxy)-N- methylpicolinamide | 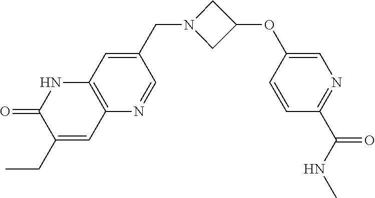 [SMILES: CCc1cc2ncc(CN3CC(Oc4ccc(C(=O)NC)nc4)C3)cc2[nH]c1=O] |
| 26 | 5-((1-((7-ethyl-6-oxo-5,6-dihydro-1,5- naphthyridin-3-yl)methyl)azetidin-3- yl)methoxy)-N-methylpicolinamide | 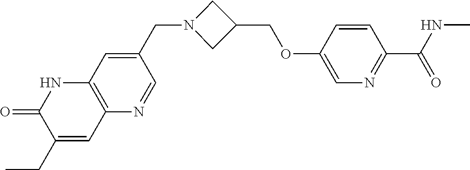 [SMILES: CCc1cc2ncc(CN3CC(COc4ccc(C(=O)NC)nc4)C3)cc2[nH]c1=O] |
| 27 | 5-(4-((7-ethyl-6-oxo-5,6-dihydro-1,5- naphthyridin-3-yl)methyl)-4-hydroxypiperidin- 1-yl)-N-methylpicolinamide | 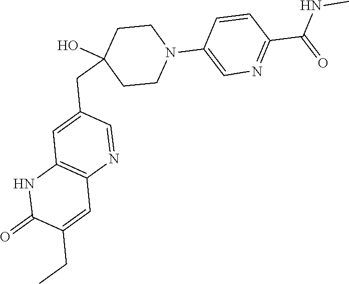 [SMILES: CCc1cc2ncc(CC3(O)CCN(c4ccc(C(=O)NC)nc4)CC3)cc2[nH]c1=O] |
| 28 | 5-(4-((7-ethyl-6-oxo-5,6-dihydro-1,5- naphthyridin-3-yl)methyl)-4-fluoropiperidin-1- yl)-N-methylpicolinamide | 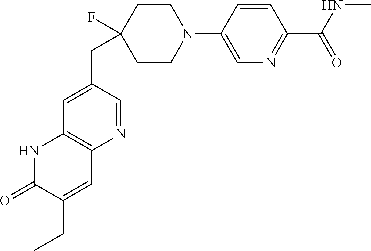 [SMILES: CCc1cc2ncc(CC3(F)CCN(c4ccc(C(=O)NC)nc4)CC3)cc2[nH]c1=O] |
| 29 | 5-((3-(((7-ethyl-6-oxo-5,6-dihydro-1,5- naphthyridin-3- yl)methyl)amino)cyclobutyl)amino)-N- methylpicolinamide | 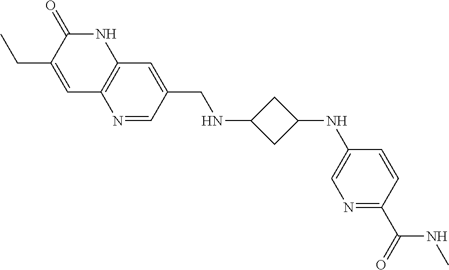 [SMILES: CCc1cc2ncc(CNC3CC(Nc4ccc(C(=O)NC)nc4)C3)cc2[nH]c1=O] |
| 30 | 5-((3-(((7-ethyl-6-oxo-5,6-dihydro-1,5- naphthyridin-3- yl)methyl)amino)bicyclo[1.1.1]pentan-1- yl)amino)-N-methylpicolinamide | 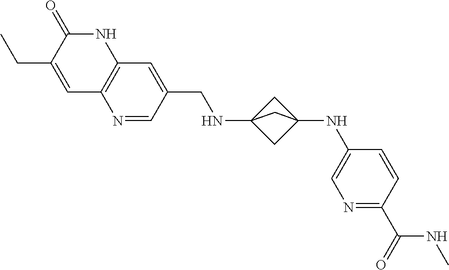 [SMILES: CCc1cc2ncc(CNC34CC(Nc5ccc(C(=O)NC)nc5)(C3)C4)cc2[nH]c1=O] |
| 31 | 5-((4-(((7-ethyl-6-oxo-5,6-dihydro-1,5- naphthyridin-3- yl)methyl)amino)cyclohexyl)amino)-N- methylpicolinamide | 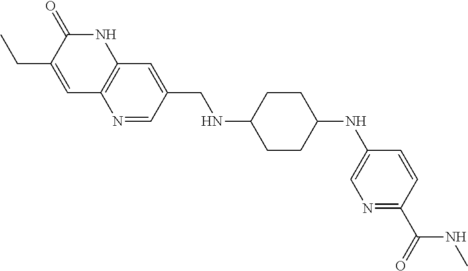 [SMILES: CCc1cc2ncc(CNC3CCC(Nc4ccc(C(=O)NC)nc4)CC3)cc2[nH]c1=O] |
| 32 | 5-((4-(((7-ethyl-6-oxo-5,6-dihydro-1,5- naphthyridin-3- yl)methyl)amino)bicyclo[2.2.2]octan-1- yl)amino)-N-methylpicolinamide | 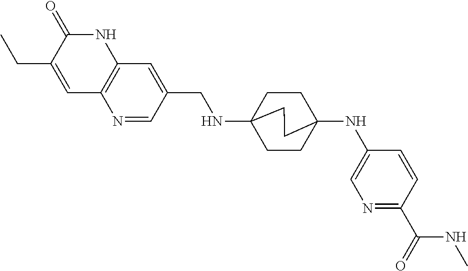 [SMILES: CCc1cc2ncc(CNC34CCC(Nc5ccc(C(=O)NC)nc5)(CC3)CC4)cc2[nH]c1=O] |
| 33 | 5-((4-(((7-ethyl-6-oxo-5,6-dihydro-1,5- naphthyridin-3- yl)methyl)amino)bicyclo[2.2.1]heptan-1- yl)amino)-N-methylpicolinamide | 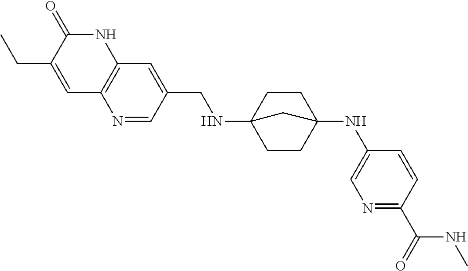 [SMILES: CCc1cc2ncc(CNC34CCC(Nc5ccc(C(=O)NC)nc5)(CC3)C4)cc2[nH]c1=O] |
| 34 | 5-(((2-(((7-ethyl-6-oxo-5,6-dihydro-1,5- naphthyridin-3- yl)methyl)amino)cyclopropyl)methyl)amino)-N- methylpicolinamide | 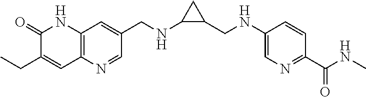 [SMILES: CCc1cc2ncc(CNC3CC3CNc3ccc(C(=O)NC)nc3)cc2[nH]c1=O] |
| 35 | 5-(4-(((7-ethyl-6-oxo-5,6-dihydro-1,5- naphthyridin-3-yl)methyl)amino)piperidin-1-yl)- N-methylpicolinamide | 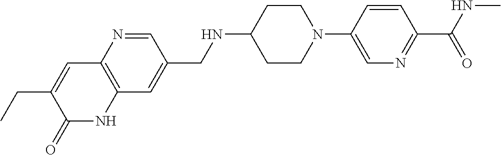 [SMILES: CCc1cc2ncc(CNC3CCN(c4ccc(C(=O)NC)nc4)CC3)cc2[nH]c1=O] |
| 36 | 6-fluoro-5-(6-((5-fluoro-2-methyl-3-oxo-3,4- dihydroquinoxalin-6-yl)methyl)-3,6- diazabicyclo[3.1.1]heptan-3-yl)-N- methylpicolinamide | 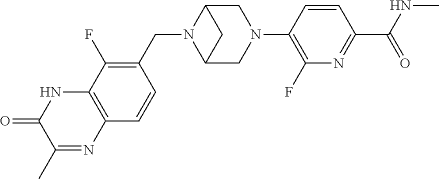 [SMILES: CNC(=O)c1ccc(N2CC3CC(C2)N3Cc2ccc3nc(C)c(=O)[nH]c3c2F)c(F)n1] |
| 37 | 6-fluoro-5-(3-((5-fluoro-2-methyl-3-oxo-3,4- dihydroquinoxalin-6-yl)methyl)-3,6- diazabicyclo[3.1.1]heptan-6-yl)-N- methylpicolinamide | 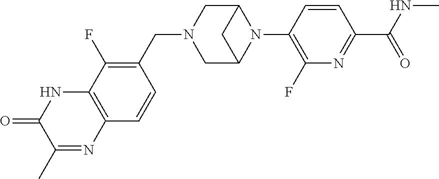 [SMILES: CNC(=O)c1ccc(N2C3CC2CN(Cc2ccc4nc(C)c(=O)[nH]c4c2F)C3)c(F)n1] |
| 38 | 6-fluoro-5-(8-((5-fluoro-2-methyl-3-oxo-3,4- dihydroquinoxalin-6-yl)methyl)-3,8- diazabicyclo[3.2.1]octan-3-yl)-N- methylpicolinamide | 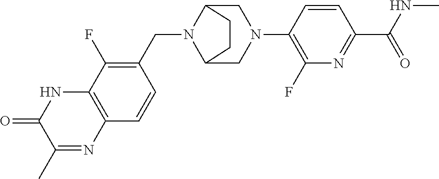 [SMILES: CNC(=O)c1ccc(N2CC3CCC(C2)N3Cc2ccc3nc(C)c(=O)[nH]c3c2F)c(F)n1] |
| 39 | 6-fluoro-5-(5-((5-fluoro-2-methyl-3-oxo-3,4- dihydroquinoxalin-6-yl)methyl)-2,5- diazabicyclo[2.2.2]octan-2-yl)-N- methylpicolinamide | 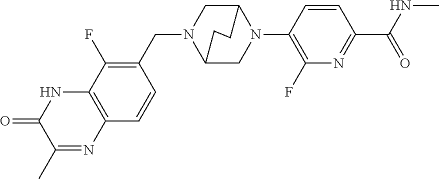 [SMILES: CNC(=O)c1ccc(N2CC3CCC2CN3Cc2ccc3nc(C)c(=O)[nH]c3c2F)c(F)n1] |
| 40 | 6-fluoro-5-(5-((5-fluoro-2-methyl-3-oxo-3,4- dihydroquinoxalin-6-yl)methyl)-2,5- diazabicyclo[2.2.1]heptan-2-yl)-N- methylpicolinamide | 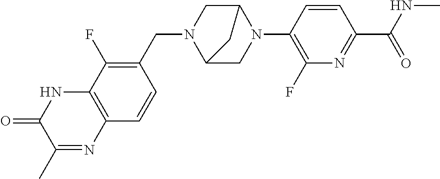 [SMILES: CNC(=O)c1ccc(N2CC3CC2CN3Cc2ccc3nc(C)c(=O)[nH]c3c2F)c(F)n1] |
| 41 | 6-fluoro-5-((1-((5-fluoro-2-methyl-3-oxo-3,4- dihydroquinoxalin-6-yl)methyl)piperidin-4- yl)amino)-N-methylpicolinamide | 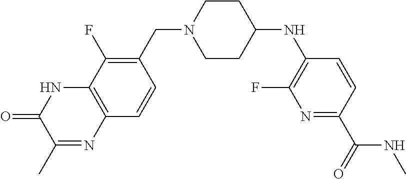 [SMILES: CNC(=O)c1ccc(NC2CCN(Cc3ccc4nc(C)c(=O)[nH]c4c3F)CC2)c(F)n1] |
| 42 | 6-fluoro-5-(4-((5-fluoro-2-methyl-3-oxo-3,4- dihydroquinoxalin-6-yl)methyl)-1,4-diazepan-1- yl)-N-methylpicolinamide | 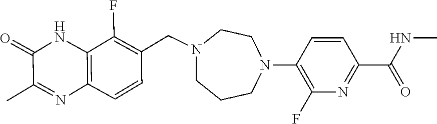 [SMILES: CNC(=O)c1ccc(N2CCCN(Cc3ccc4nc(C)c(=O)[nH]c4c3F)CC2)c(F)n1] |
| 43 | 6-fluoro-5-(5-((5-fluoro-2-methyl-3-oxo-3,4- dihydroquinoxalin-6- yl)methyl)hexahydropyrrolo[3,4-c]pyrrol-2(1H)- yl)-N-methylpicolinamide | 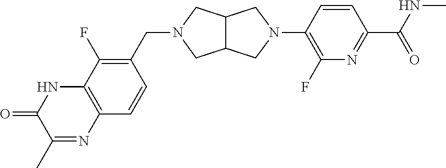 [SMILES: CNC(=O)c1ccc(N2CC3CN(Cc4ccc5nc(C)c(=O)[nH]c5c4F)CC3C2)c(F)n1] |
| 44 | 6-fluoro-5-(3-((5-fluoro-2-methyl-3-oxo-3,4- dihydroquinoxalin-6-yl)methyl)-3,8- diazabicyclo[3.2.1]octan-8-yl)-N- methylpicolinamide | 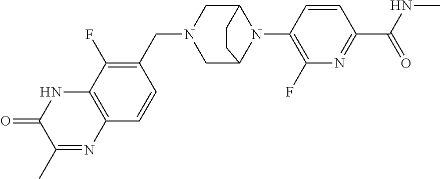 [SMILES: CNC(=O)c1ccc(N2C3CCC2CN(Cc2ccc4nc(C)c(=O)[nH]c4c2F)C3)c(F)n1] |
| 45 | 6-fluoro-5-(6-((5-fluoro-2-methyl-3-oxo-3,4- dihydroquinoxalin-6-yl)methyl)-2,6- diazaspiro[3.3]heptan-2-yl)-N- methylpicolinamide | 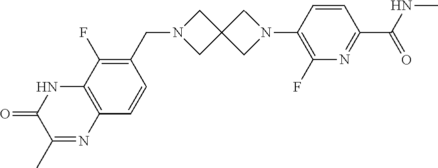 [SMILES: CNC(=O)c1ccc(N2CC3(CN(Cc4ccc5nc(C)c(=O)[nH]c5c4F)C3)C2)c(F)n1] |
| 46 | 6-fluoro-5-((3-(((5-fluoro-2-methyl-3-oxo-3,4- dihydroquinoxalin-6- yl)methyl)amino)cyclobutyl)amino)-N- methylpicolinamide | 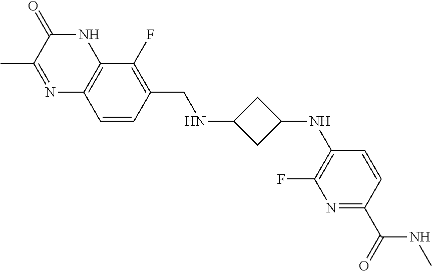 |
| 47 | 6-fluoro-5-((3-(((5-fluoro-2-methyl-3-oxo-3,4- dihydroquinoxalin-6- yl)methyl)amino)bicyclo[1.1.1]pentan-1- yl)amino)-N-methylpicolinamide | 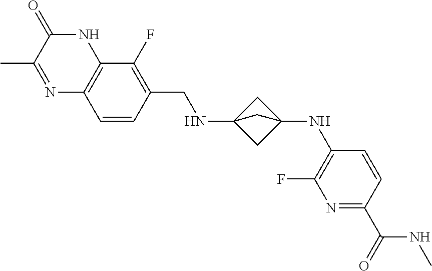 |
| 48 | 6-fluoro-5-((4-(((5-fluoro-2-methyl-3-oxo-3,4- dihydroquinoxalin-6- yl)methyl)amino)cyclohexyl)amino)-N- methylpicolinamide | 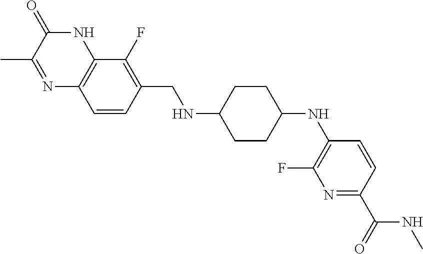 |
| 49 | 6-fluoro-5-((4-(((5-fluoro-2-methyl-3-oxo-3,4- dihydroquinoxalin-6- yl)methyl)amino)bicyclo[2.2.2]octan-1- yl)amino)-N-methylpicolinamide | 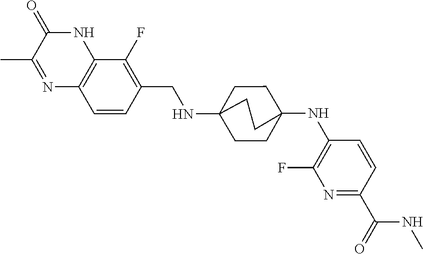 |
| 50 | 6-fluoro-5-((4-(((5-fluoro-2-methyl-3-oxo-3,4- dihydroquinoxalin-6- yl)methyl)amino)bicyclo[2.2.1]heptan-1- yl)amino)-N-methylpicolinamide | 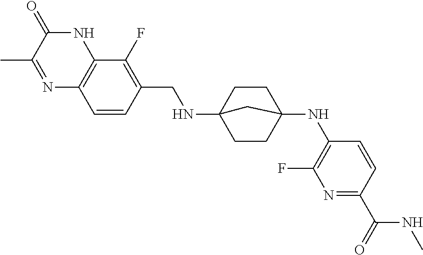 |
| 51 | 6-fluoro-5-(5-((5-fluoro-2-methyl-3-oxo-3,4- dihydroquinoxalin-6-yl)methyl)-2-oxa-5,8- diazaspiro[3.5]nonan-8-yl)-N- methylpicolinamide | 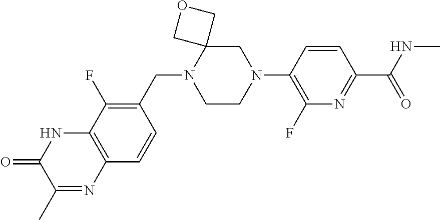 [SMILES: CNC(=O)c1ccc(N2CCN(Cc3ccc4nc(C)c(=O)[nH]c4c3F)C3(COC3)C2)c(F)n1] |
| 52 | 6-fluoro-5-((2-((5-fluoro-2-methyl-3-oxo-3,4- dihydroquinoxalin-6- yl)methyl)octahydrocyclopenta[c]pyrrol-5- yl)amino)-N-methylpicolinamide | 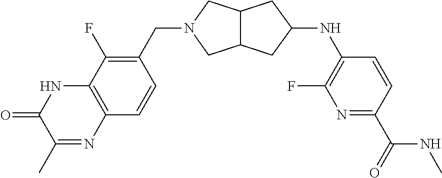 [SMILES: CNC(=O)c1ccc(NC2CC3CN(Cc4ccc5nc(C)c(=O)[nH]c5c4F)CC3C2)c(F)n1] |
| 53 | 6-fluoro-5-((3-((5-fluoro-2-methyl-3-oxo-3,4- dihydroquinoxalin-6-yl)methyl)-3- azabicyclo[3.1.0]hexan-6-yl)amino)-N- methylpicolinamide | 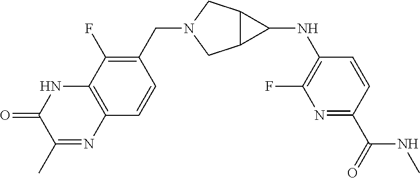 [SMILES: CNC(=O)c1ccc(NC2C3CN(Cc4ccc5nc(C)c(=O)[nH]c5c4F)CC32)c(F)n1] |
| 54 | 6-fluoro-5-((1-((5-fluoro-2-methyl-3-oxo-3,4- dihydroquinoxalin-6-yl)methyl)azetidin-3- yl)amino)-N-methylpicolinamide | 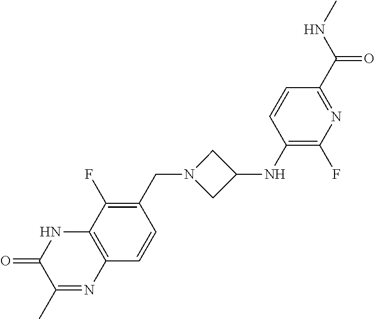 [SMILES: CNC(=O)c1ccc(NC2CN(Cc3ccc4nc(C)c(=O)[nH]c4c3F)C2)c(F)n1] |
| 55 | 6-fluoro-5-(((2-(((5-fluoro-2-methyl-3-oxo-3,4- dihydroquinoxalin-6- yl)methyl)amino)cyclopropyl)methyl)amino)-N- methylpicolinamide | 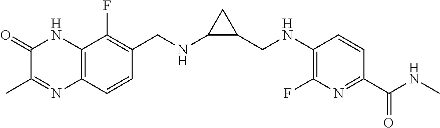 [SMILES: CNC(=O)c1ccc(NCC2CC2NCc2ccc3nc(C)c(=O)[nH]c3c2F)c(F)n1] |
| 56 | 6-fluoro-5-(4-((5-fluoro-2-methyl-3-oxo-3,4- dihydroquinoxalin-6-yl)methyl)-2-oxo-1,4- diazepan-1-yl)-N-methylpicolinamide | 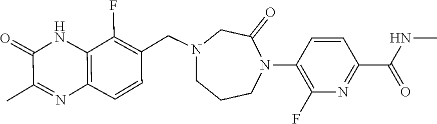 [SMILES: CNC(=O)c1ccc(N2CCCN(Cc3ccc4nc(C)c(=O)[nH]c4c3F)CC2=O)c(F)n1] |
| 57 | 6-fluoro-5-(4-((5-fluoro-2-methyl-3-oxo-3,4- dihydroquinoxalin-6-yl)methyl)-7-oxo-1,4- diazepan-1-yl)-N-methylpicolinamide | 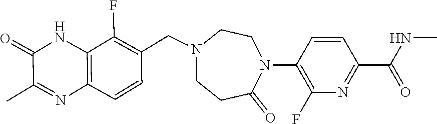 [SMILES: CNC(=O)c1ccc(N2CCN(Cc3ccc4nc(C)c(=O)[nH]c4c3F)CCC2=O)c(F)n1] |
| 58 | 6-fluoro-5-(4-((5-fluoro-2-methyl-3-oxo-3,4- dihydroquinoxalin-6-yl)methyl)-4,7- diazaspiro[2.5]octan-7-yl)-N- methylpicolinamide | 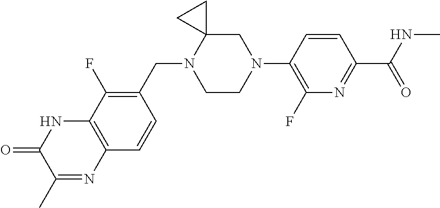 [SMILES: CNC(=O)c1ccc(N2CCN(Cc3ccc4nc(C)c(=O)[nH]c4c3F)C3(CC3)C2)c(F)n1] |
| 59 | 6-fluoro-5-(5-((5-fluoro-2-methyl-3-oxo-3,4- dihydroquinoxalin-6-yl)methyl)-5,8- diazaspiro[3.5]nonan-8-yl)-N- methylpicolinamide | 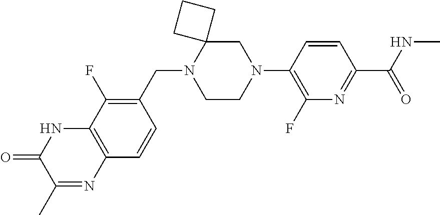 [SMILES: CNC(=O)c1ccc(N2CCN(Cc3ccc4nc(C)c(=O)[nH]c4c3F)C3(CCC3)C2)c(F)n1] |
| 60 | 6-fluoro-5-(6-((5-fluoro-2-methyl-3-oxo-3,4- dihydroquinoxalin-6-yl)methyl)-2-oxa-6,9- diazaspiro[4.5]decan-9-yl)-N- methylpicolinamide | 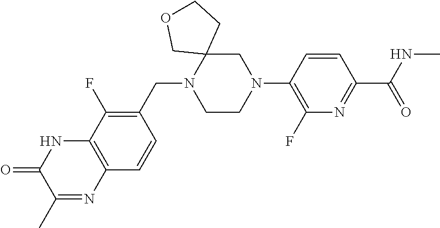 [SMILES: CNC(=O)c1ccc(N2CCN(Cc3ccc4nc(C)c(=O)[nH]c4c3F)C3(CCOC3)C2)c(F)n1] |
| 61 | 6-fluoro-5-(8-((5-fluoro-2-methyl-3-oxo-3,4- dihydroquinoxalin-6-yl)methyl)-2-oxa-5,8- diazaspiro[3.5]nonan-5-yl)-N- methylpicolinamide | 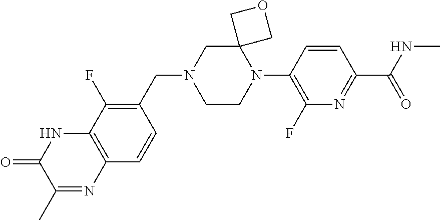 [SMILES: CNC(=O)c1ccc(N2CCN(Cc3ccc4nc(C)c(=O)[nH]c4c3F)CC23COC3)c(F)n1] |
| 62 | 6-fluoro-5-(7-((5-fluoro-2-methyl-3-oxo-3,4- dihydroquinoxalin-6-yl)methyl)-4,7- diazaspiro[2.5]octan-4-yl)-N- methylpicolinamide | 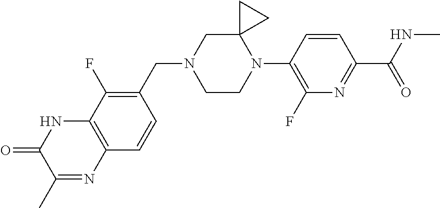 [SMILES: CNC(=O)c1ccc(N2CCN(Cc3ccc4nc(C)c(=O)[nH]c4c3F)CC23CC3)c(F)n1] |
| 63 | 6-fluoro-5-(8-((5-fluoro-2-methyl-3-oxo-3,4- dihydroquinoxalin-6-yl)methyl)-5,8- diazaspiro[3.5]nonan-5-yl)-N- methylpicolinamide | 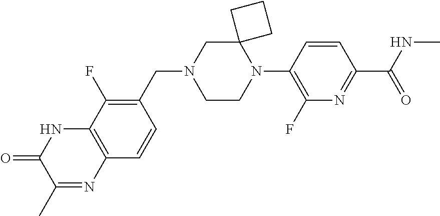 [SMILES: CNC(=O)c1ccc(N2CCN(Cc3ccc4nc(C)c(=O)[nH]c4c3F)CC23CCC3)c(F)n1] |
| 64 | 6-fluoro-5-(9-((5-fluoro-2-methyl-3-oxo-3,4- dihydroquinoxalin-6-yl)methyl)-2-oxa-6,9- diazaspiro[4.5]decan-6-yl)-N- methylpicolinamide | 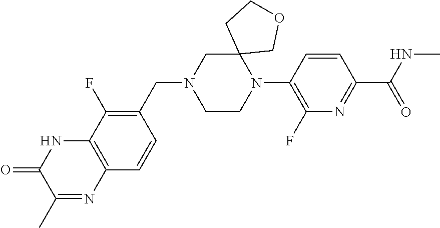 [SMILES: CNC(=O)c1ccc(N2CCN(Cc3ccc4nc(C)c(=O)[nH]c4c3F)CC23CCOC3)c(F)n1] |
| 65 | 6-fluoro-5-(((1-((5-fluoro-2-methyl-3-oxo-3,4- dihydroquinoxalin-6-yl)methyl)azetidin-3- yl)methyl)amino)-N-methylpicolinamide | 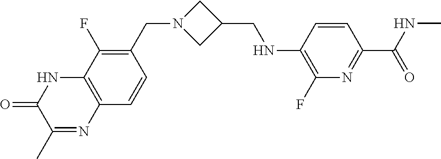 [SMILES: CNC(=O)c1ccc(NCC2CN(Cc3ccc4nc(C)c(=O)[nH]c4c3F)C2)c(F)n1] |
| 66 | 6-fluoro-5-((1-((5-fluoro-2-methyl-3-oxo-3,4- dihydroquinoxalin-6-yl)methyl)azetidin-3- yl)oxy)-N-methylpicolinamide | 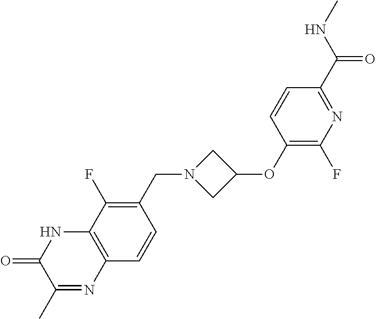 [SMILES: CNC(=O)c1ccc(OC2CN(Cc3ccc4nc(C)c(=O)[nH]c4c3F)C2)c(F)n1] |
| 67 | 6-fluoro-5-((1-((5-fluoro-2-methyl-3-oxo-3,4- dihydroquinoxalin-6-yl)methyl)azetidin-3- yl)methoxy)-N-methylpicolinamide | 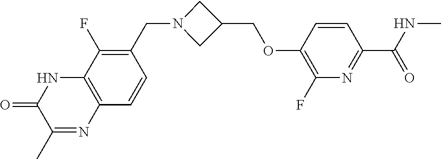 [SMILES: CNC(=O)c1ccc(OCC2CN(Cc3ccc4nc(C)c(=O)[nH]c4c3F)C2)c(F)n1] |
| 68 | 6-fluoro-5-(4-((5-fluoro-2-methyl-3-oxo-3,4- dihydroquinoxalin-6-yl)methyl)-4- hydroxypiperidin-1-yl)-N-methylpicolinamide | 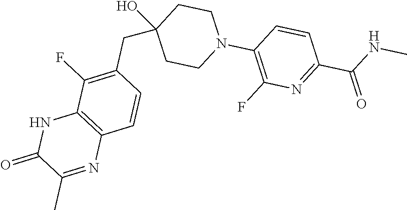 [SMILES: CNC(=O)c1ccc(N2CCC(O)(Cc3ccc4nc(C)c(=O)[nH]c4c3F)CC2)c(F)n1] |
| 69 | 6-fluoro-5-(4-fluoro-4-((5-fluoro-2-methyl-3- oxo-3,4-dihydroquinoxalin-6- yl)methyl)piperidin-1-yl)-N-methylpicolinamide | 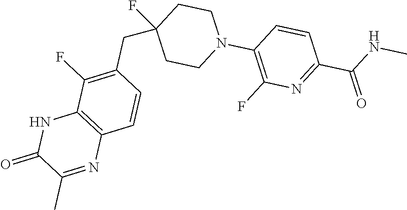 [SMILES: CNC(=O)c1ccc(N2CCC(F)(Cc3ccc4nc(C)c(=O)[nH]c4c3F)CC2)c(F)n1] |
| 70 | 6-fluoro-5-(4-(((5-fluoro-2-methyl-3-oxo-3,4- dihydroquinoxalin-6-yl)methyl)amino)piperidin- 1-yl)-N-methylpicolinamide | 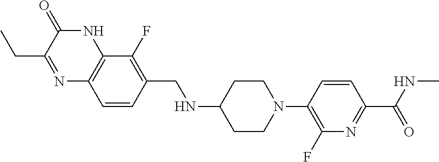 [SMILES: CCc1nc2ccc(CNC3CCN(c4ccc(C(=O)NC)nc4F)CC3)c(F)c2[nH]c1=O] |
| 71 | 3-ethyl-7-(((3-((4- fluorophenyl)amino)cyclobutyl)amino)methyl) quinoxalin-2(1H)-one | 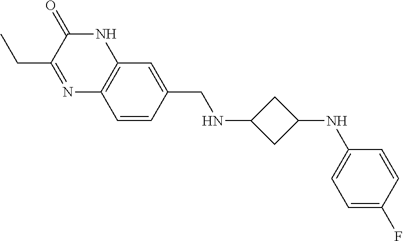 [SMILES: CCc1nc2ccc(CNC3CC(Nc4ccc(F)cc4)C3)cc2[nH]c1=O] |
| 72 | 6-((3-(((2-ethyl-3-oxo-3,4-dihydroquinoxalin-6- yl)methyl)amino)cyclobutyl)amino)-N- methylpyridazine-3-carboxamide | 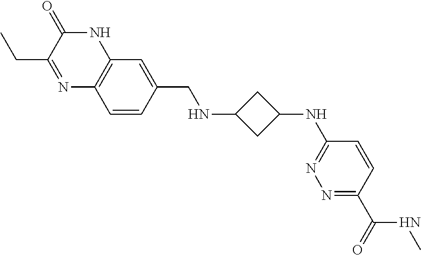 [SMILES: CCc1nc2ccc(CNC3CC(Nc4ccc(C(=O)NC)nn4)C3)cc2[nH]c1=O] |
| 73 | 5-((3-(((2-ethyl-3-oxo-3,4-dihydroquinoxalin-6- yl)methyl)amino)cyclobutyl)amino)-6-fluoro-N- methylpicolinamide | 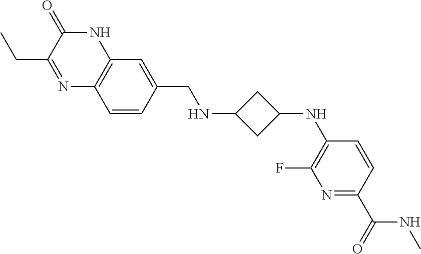 [SMILES: CCc1nc2ccc(CNC3CC(Nc4ccc(C(=O)NC)nc4F)C3)cc2[nH]c1=O] |
| 74 | 5-((3-(((2-ethyl-3-oxo-3,4-dihydroquinoxalin-6- yl)methyl)amino)cyclobutyl)amino)-N- methylpicolinamide | 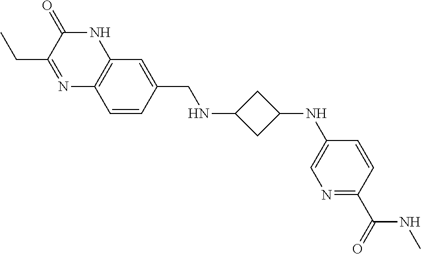 [SMILES: CCc1nc2ccc(CNC3CC(Nc4ccc(C(=O)NC)nc4)C3)cc2[nH]c1=O] |
| 75 | 3-ethyl-7-((6-(4-fluorophenyl)-2,6- diazaspiro[3.3]heptan-2-yl)methyl)quinoxalin- 2(1H)-one | 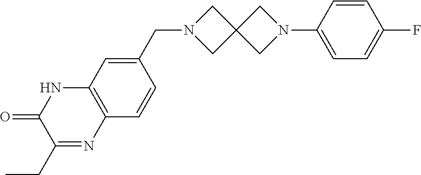 [SMILES: CCc1nc2ccc(CN3CC4(C3)CN(c3ccc(F)cc3)C4)cc2[nH]c1=O] |
| 76 | 6-(6-((2-ethyl-3-oxo-3,4-dihydroquinoxalin-6- yl)methyl)-2,6-diazaspiro[3.3]heptan-2-yl)-N- methylpyridazine-3-carboxamide | 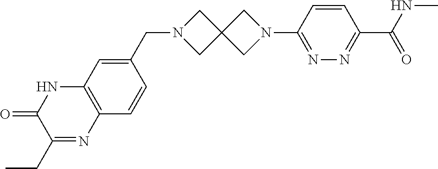 [SMILES: CCc1nc2ccc(CN3CC4(C3)CN(c3ccc(C(=O)NC)nn3)C4)cc2[nH]c1=O] |
| 77 | 5-(6-((2-ethyl-3-oxo-3,4-dihydroquinoxalin-6- yl)methyl)-2,6-diazaspiro[3.3]heptan-2-yl)-6- fluoro-N-methylpicolinamide | 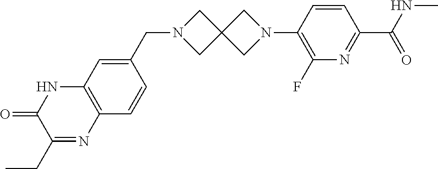 [SMILES: CCc1nc2ccc(CN3CC4(C3)CN(c3ccc(C(=O)NC)nc3F)C4)cc2[nH]c1=O] |
| 78 | 5-(6-((2-ethyl-3-oxo-3,4-dihydroquinoxalin-6- yl)methyl)-2,6-diazaspiro[3.3]heptan-2-yl)-N- methylpicolinamide | 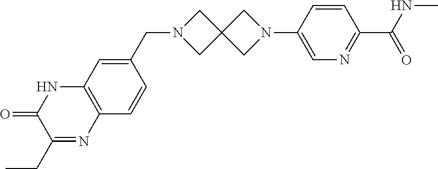 [SMILES: CCc1nc2ccc(CN3CC4(C3)CN(c3ccc(C(=O)NC)nc3)C4)cc2[nH]c1=O] |
| 79 | 3-ethyl-7-(((3-((4- fluorophenyl)amino)cyclobutyl)amino)methyl) pyrido[2,3-b]pyrazin-2(1H)-one | 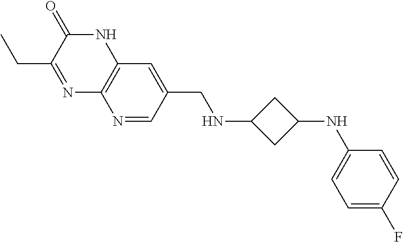 [SMILES: CCc1nc2ncc(CNC3CC(Nc4ccc(F)cc4)C3)cc2[nH]c1=O] |
| 80 | 6-((3-(((3-ethyl-2-oxo-1,2-dihydropyrido[2,3- b]pyrazin-7- yl)methyl)amino)cyclobutyl)amino)-N- methylpyridazine-3-carboxamide | 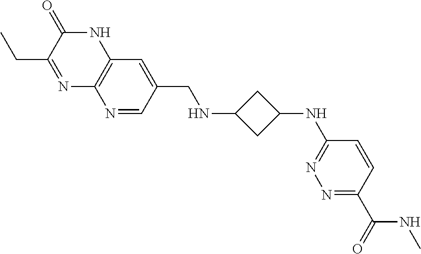 [SMILES: CCc1nc2ncc(CNC3CC(Nc4ccc(C(=O)NC)nn4)C3)cc2[nH]c1=O] |
| 81 | 5-((3-(((3-ethyl-2-oxo-1,2-dihydropyrido[2,3- b]pyrazin-7- yl)methyl)amino)cyclobutyl)amino)-6-fluoro-N- methylpicolinamide | 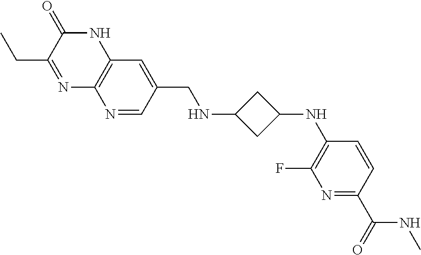 [SMILES: CCc1nc2ncc(CNC3CC(Nc4ccc(C(=O)NC)nc4F)C3)cc2[nH]c1=O] |
| 82 | 5-((3-(((3-ethyl-2-oxo-1,2-dihydropyrido[2,3- b]pyrazin-7- yl)methyl)amino)cyclobutyl)amino)-N- methylpicolinamide | 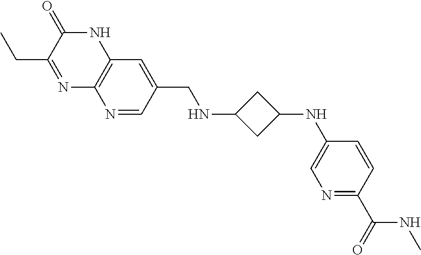 [SMILES: CCc1nc2ncc(CNC3CC(Nc4ccc(C(=O)NC)nc4)C3)cc2[nH]c1=O] |
| 83 | 3-ethyl-7-((6-(4-fluorophenyl)-2,6- diazaspiro[3.3]heptan-2-yl)methyl)pyrido[2,3- b]pyrazin-2(1H)-one | 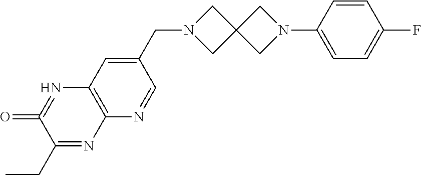 |
| 84 | 6-(6-((3-ethyl-2-oxo-1,2-dihydropyrido[2,3- b]pyrazin-7-yl)methyl)-2,6- diazaspiro[3.3]heptan-2-yl)-N- methylpyridazine-3-carboxamide | 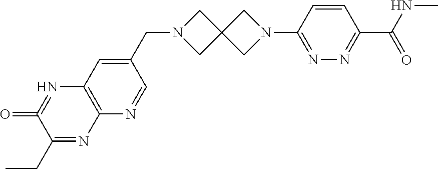 |
| 85 | 5-(6-((3-ethyl-2-oxo-1,2-dihydropyrido[2,3- b]pyrazin-7-yl)methyl)-2,6- diazaspiro[3.3]heptan-2-yl)-6-fluoro-N- methylpicolinamide | 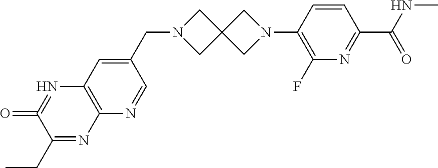 |
| 86 | 5-(6-((3-ethyl-2-oxo-1,2-dihydropyrido[2,3- b]pyrazin-7-yl)methyl)-2,6- diazaspiro[3.3]heptan-2-yl)-N- methylpicolinamide | 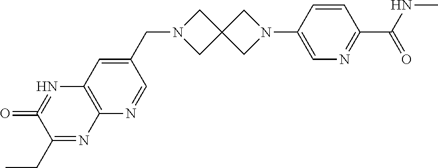 |
| 87 | 3-ethyl-7-(((3-((4- fluorophenyl)amino)cyclobutyl)amino)methyl) pyrido[3,4-b]pyrazin-2(1H)-one | 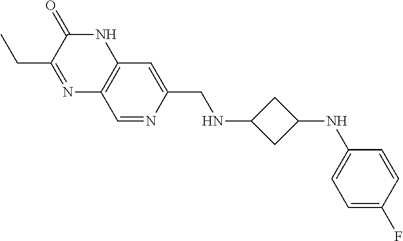 [SMILES: CCc1nc2cnc(CNC3CC(Nc4ccc(F)cc4)C3)cc2[nH]c1=O] |
| 88 | 6-((3-(((3-ethyl-2-oxo-1,2-dihydropyrido[3,4- b]pyrazin-7- yl)methyl)amino)cyclobutyl)amino)-N- methylpyridazine-3-carboxamide | 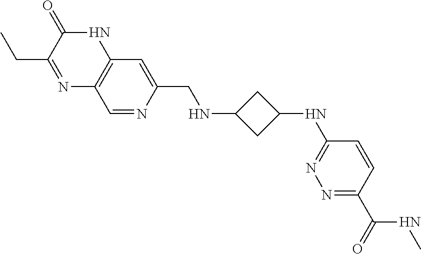 [SMILES: CCc1nc2cnc(CNC3CC(Nc4ccc(C(=O)NC)nn4)C3)cc2[nH]c1=O] |
| 89 | 5-((3-(((3-ethyl-2-oxo-1,2-dihydropyrido[3,4- b]pyrazin-7- yl)methyl)amino)cyclobutyl)amino)-6-fluoro-N- methylpicolinamide | 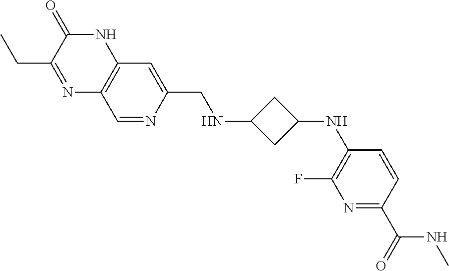 [SMILES: CCc1nc2cnc(CNC3CC(Nc4ccc(C(=O)NC)nc4F)C3)cc2[nH]c1=O] |
| 90 | 5-((3-(((3-ethyl-2-oxo-1,2-dihydropyrido[3,4- b]pyrazin-7- yl)methyl)amino)cyclobutyl)amino)-N- methylpicolinamide | 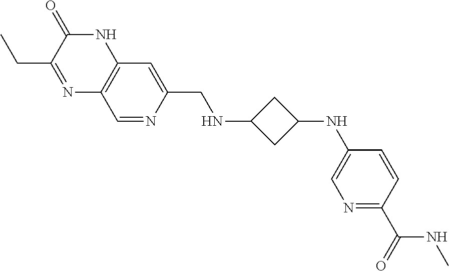 [SMILES: CCc1nc2cnc(CNC3CC(Nc4ccc(C(=O)NC)nc4)C3)cc2[nH]c1=O] |
| 91 | 3-ethyl-7-((6-(4-fluorophenyl)-2,6- diazaspiro[3.3]heptan-2-yl)methyl)pyrido[3,4- b]pyrazin-2(1H)-one | 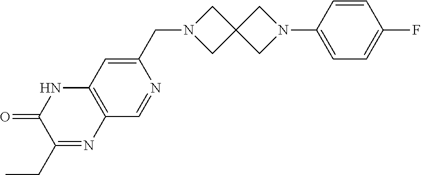 [SMILES: CCc1nc2cnc(CN3CC4(C3)CN(c3ccc(F)cc3)C4)cc2[nH]c1=O] |
| 92 | 6-(6-((3-ethyl-2-oxo-1,2-dihydropyrido[3,4- b]pyrazin-7-yl)methyl)-2,6- diazaspiro[3.3]heptan-2-yl)-N- methylpyridazine-3-carboxamide | 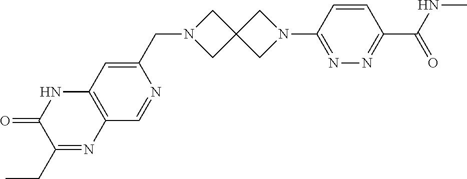 [SMILES: CCc1nc2cnc(CN3CC4(C3)CN(c3ccc(C(=O)NC)nn3)C4)cc2[nH]c1=O] |
| 93 | 5-(6-((3-ethyl-2-oxo-1,2-dihydropyrido[3,4- b]pyrazin-7-yl)methyl)-2,6- diazaspiro[3.3]heptan-2-yl)-6-fluoro-N- methylpicolinamide | 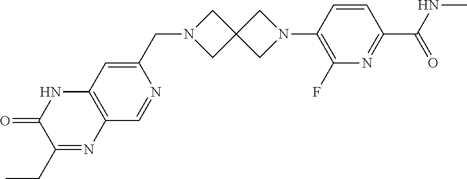 [SMILES: CCc1nc2cnc(CN3CC4(C3)CN(c3ccc(C(=O)NC)nc3F)C4)cc2[nH]c1=O] |
| 94 | 5-(6-((3-ethyl-2-oxo-1,2-dihydropyrido[3,4- b]pyrazin-7-yl)methyl)-2,6- diazaspiro[3.3]heptan-2-yl)-N- methylpicolinamide | 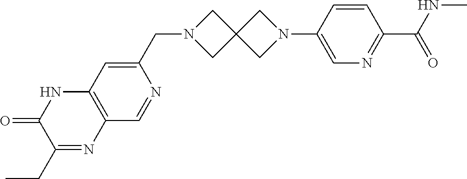 [SMILES: CCc1nc2cnc(CN3CC4(C3)CN(c3ccc(C(=O)NC)nc3)C4)cc2[nH]c1=O] |
| 95 | 3-ethyl-7-(((3-((4- fluorophenyl)amino)cyclobutyl)amino)methyl)q uinolin-2(1H)-one | 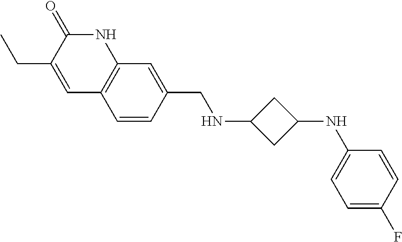 [SMILES: CCc1cc2ccc(CNC3CC(Nc4ccc(F)cc4)C3)cc2[nH]c1=O] |
| 96 | 6-((3-(((3-ethyl-2-oxo-1,2-dihydroquinolin-7- yl)methyl)amino)cyclobutyl)amino)-N- methylpyridazine-3-carboxamide | 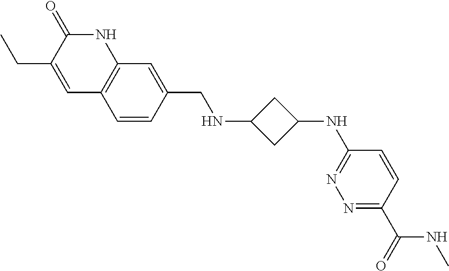 [SMILES: CCc1cc2ccc(CNC3CC(Nc4ccc(C(=O)NC)nn4)C3)cc2[nH]c1=O] |
| 97 | 5-((3-(((3-ethyl-2-oxo-1,2-dihydroquinolin-7- yl)methyl)amino)cyclobutyl)amino)-6-fluoro-N- methylpicolinamide | 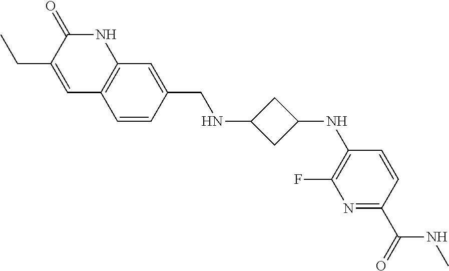 [SMILES: CCc1cc2ccc(CNC3CC(Nc4ccc(C(=O)NC)nc4F)C3)cc2[nH]c1=O] |
| 98 | 5-((3-(((3-ethyl-2-oxo-1,2-dihydroquinolin-7- yl)methyl)amino)cyclobutyl)amino)-N- methylpicolinamide | 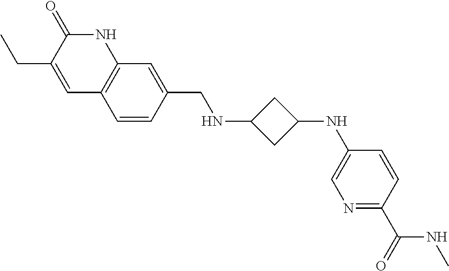 [SMILES: CCc1cc2ccc(CNC3CC(Nc4ccc(C(=O)NC)nc4)C3)cc2[nH]c1=O] |
| 99 | 3-ethyl-7-((6-(4-fluorophenyl)-2,6- diazaspiro[3.3]heptan-2-yl)methyl)quinolin- 2(1H)-one | 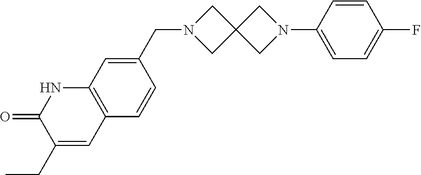 [SMILES: CCc1cc2ccc(CN3CC4(C3)CN(c3ccc(F)cc3)C4)cc2[nH]c1=O] |
| 100 | 6-(6-((3-ethyl-2-oxo-1,2-dihydroquinolin-7- yl)methyl)-2,6-diazaspiro[3.3]heptan-2-yl)-N- methylpyridazine-3-carboxamide | 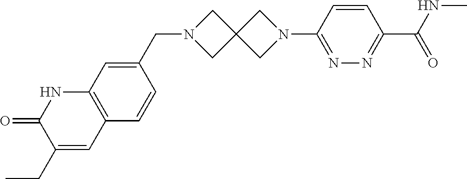 [SMILES: CCc1cc2ccc(CN3CC4(C3)CN(c3ccc(C(=O)NC)nn3)C4)cc2[nH]c1=O] |
| 101 | 5-(6-((3-ethyl-2-oxo-1,2-dihydroquinolin-7- yl)methyl)-2,6-diazaspiro[3.3]heptan-2-yl)-6- fluoro-N-methylpicolinamide | 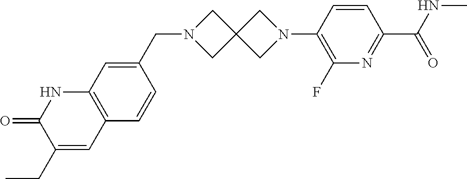 [SMILES: CCc1cc2ccc(CN3CC4(C3)CN(c3ccc(C(=O)NC)nc3F)C4)cc2[nH]c1=O] |
| 102 | 5-(6-((3-ethyl-2-oxo-1,2-dihydroquinolin-7- yl)methyl)-2,6-diazaspiro[3.3]heptan-2-yl)-N- methylpicolinamide | 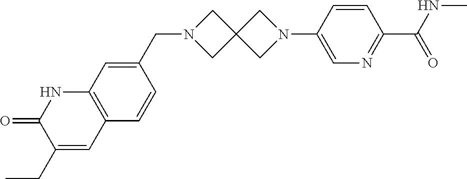 [SMILES: CCc1cc2ccc(CN3CC4(C3)CN(c3ccc(C(=O)NC)nc3)C4)cc2[nH]c1=O] |
| 103 | 3-ethyl-7-(((3-((4- fluorophenyl)amino)cyclobutyl)amino)methyl)- 1,5-naphthyridin-2(1H)-one | 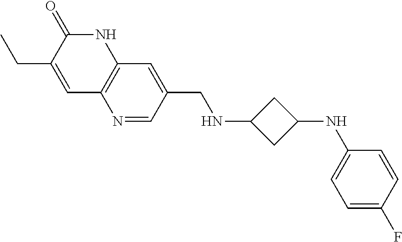 [SMILES: CCc1cc2ncc(CNC3CC(Nc4ccc(F)cc4)C3)cc2[nH]c1=O] |
| 104 | 6-((3-(((7-ethyl-6-oxo-5,6-dihydro-1,5- naphthyridin-3- yl)methyl)amino)cyclobutyl)amino)-N- methylpyridazine-3-carboxamide | 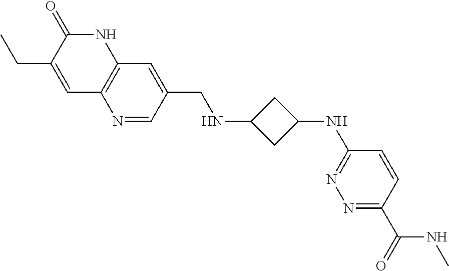 [SMILES: CCc1cc2ncc(CNC3CC(Nc4ccc(C(=O)NC)nn4)C3)cc2[nH]c1=O] |
| 105 | 5-((3-(((7-ethyl-6-oxo-5,6-dihydro-1,5- naphthyridin-3- yl)methyl)amino)cyclobutyl)amino)-6-fluoro-N- methylpicolinamide | 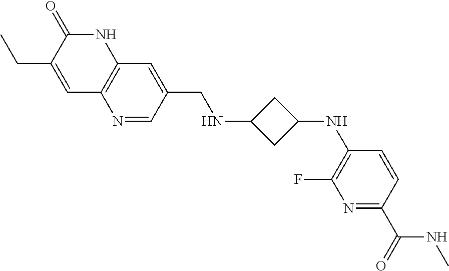 [SMILES: CCc1cc2ncc(CNC3CC(Nc4ccc(C(=O)NC)nc4F)C3)cc2[nH]c1=O] |
| 106 | 3-ethyl-7-((6-(4-fluorophenyl)-2,6- diazaspiro[3.3]heptan-2-yl)methyl)-1,5- naphthyridin-2(1H)-one | 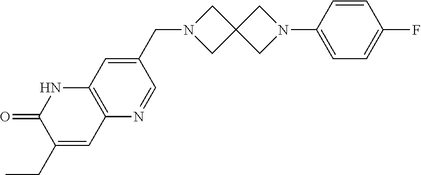 [SMILES: CCc1cc2ncc(CN3CC4(C3)CN(c3ccc(F)cc3)C4)cc2[nH]c1=O] |
| 107 | 6-(6-((7-ethyl-6-oxo-5,6-dihydro-1,5- naphthyridin-3-yl)methyl)-2,6- diazaspiro[3.3]heptan-2-yl)-N- methylpyridazine-3-carboxamide | 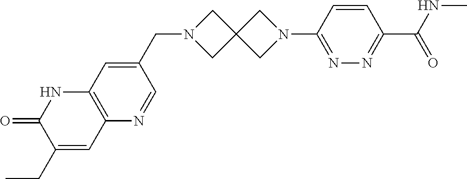 [SMILES: CCc1cc2ncc(CN3CC4(C3)CN(c3ccc(C(=O)NC)nn3)C4)cc2[nH]c1=O] |
| 108 | 5-(6-((7-ethyl-6-oxo-5,6-dihydro-1,5- naphthyridin-3-yl)methyl)-2,6- diazaspiro[3.3]heptan-2-yl)-6-fluoro-N- methylpicolinamide | 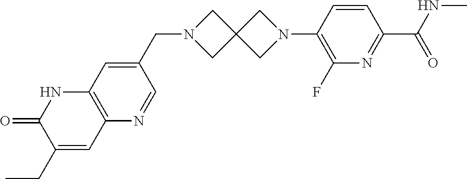 [SMILES: CCc1cc2ncc(CN3CC4(C3)CN(c3ccc(C(=O)NC)nc3F)C4)cc2[nH]c1=O] |
| 109 | 3-ethyl-7-(((3-((4- fluorophenyl)amino)cyclobutyl)amino)methyl)- 1,6-naphthyridin-2(1H)-one | 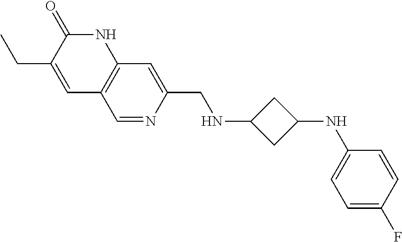 [SMILES: CCc1cc2cnc(CNC3CC(Nc4ccc(F)cc4)C3)cc2[nH]c1=O] |
| 110 | 6-((3-(((3-ethyl-2-oxo-1,2-dihydro-1,6- naphthyridin-7- yl)methyl)amino)cyclobutyl)amino)-N- methylpyridazine-3-carboxamide | 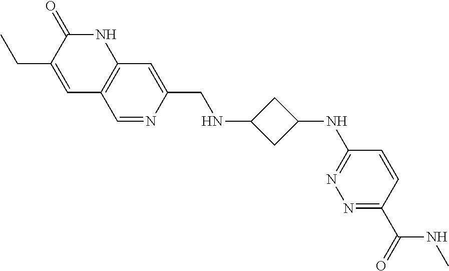 [SMILES: CCc1cc2cnc(CNC3CC(Nc4ccc(C(=O)NC)nn4)C3)cc2[nH]c1=O] |
| 111 | 5-((3-(((3-ethyl-2-oxo-1,2-dihydro-1,6- naphthyridin-7- yl)methyl)amino)cyclobutyl)amino)-6-fluoro-N- methylpicolinamide | 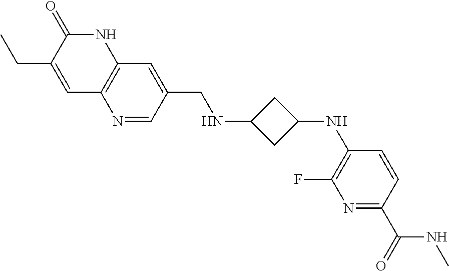 [SMILES: CCc1cc2ncc(CNC3CC(Nc4ccc(C(=O)NC)nc4F)C3)cc2[nH]c1=O] |
| 112 | 5-((3-(((3-ethyl-2-oxo-1,2-dihydro-1,6- naphthyridin-7- yl)methyl)amino)cyclobutyl)amino)-N- methylpicolinamide | 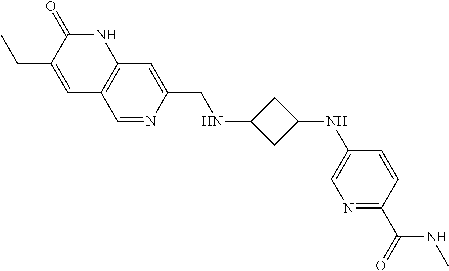 [SMILES: CCc1cc2cnc(CNC3CC(Nc4ccc(C(=O)NC)nc4)C3)cc2[nH]c1=O] |
| 113 | 3-ethyl-7-((6-(4-fluorophenyl)-2,6- diazaspiro[3.3]heptan-2-yl)methyl)-1,6- naphthyridin-2(1H)-one | 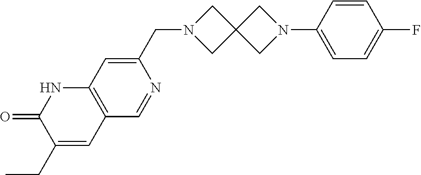 [SMILES: CCc1cc2cnc(CN3CC4(C3)CN(c3ccc(F)cc3)C4)cc2[nH]c1=O] |
| 114 | 6-(6-((3-ethyl-2-oxo-1,2-dihydro-1,6- naphthyridin-7-yl)methyl)-2,6- diazaspiro[3.3]heptan-2-yl)-N- methylpyridazine-3-carboxamide | 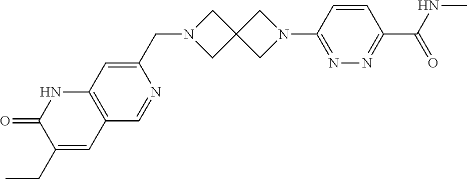 [SMILES: CCc1cc2cnc(CN3CC4(C3)CN(c3ccc(C(=O)NC)nn3)C4)cc2[nH]c1=O] |
| 115 | 5-(6-((3-ethyl-2-oxo-1,2-dihydro-1,6- naphthyridin-7-yl)methyl)-2,6- diazaspiro[3.3]heptan-2-yl)-6-fluoro-N- methylpicolinamide | 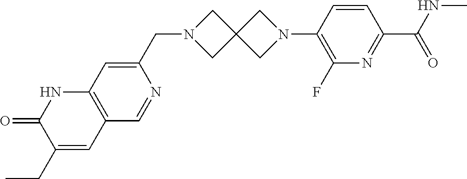 [SMILES: CCc1cc2cnc(CN3CC4(C3)CN(c3ccc(C(=O)NC)nc3F)C4)cc2[nH]c1=O] |
| 116 | 5-(6-((3-ethyl-2-oxo-1,2-dihydro-1,6- naphthyridin-7-yl)methyl)-2,6- diazaspiro[3.3]heptan-2-yl)-N- methylpicolinamide | 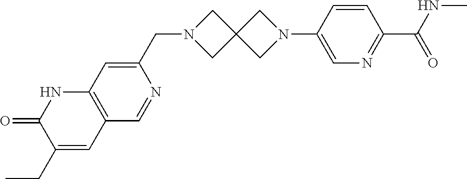 [SMILES: CCc1cc2cnc(CN3CC4(C3)CN(c3ccc(C(=O)NC)nc3)C4)cc2[nH]c1=O] |
| 117 | N-methyl-5-((3-(((2-methyl-3-oxo-3,4- dihydroquinoxalin-6- yl)methyl)amino)cyclobutyl)amino) picolinamide | 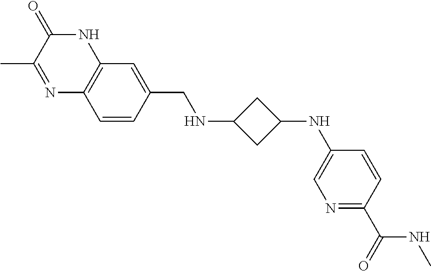 |
| 118 | N-methyl-5-(6-((2-methyl-3-oxo-3,4- dihydroquinoxalin-6-yl)methyl)-2,6- diazaspiro[3.3]heptan-2-yl)picolinamide | 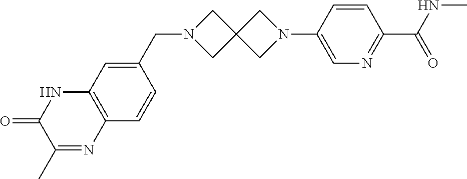 [SMILES: CNC(=O)c1ccc(N2CC3(CN(Cc4ccc5nc(C)c(=O)[nH]c5c4)C3)C2)cn1] |
| 119 | N-methyl-5-((3-(((3-oxo-2-(trifluoromethyl)- 3,4-dihydroquinoxalin-6- yl)methyl)amino)cyclobutyl)amino) picolinamide | 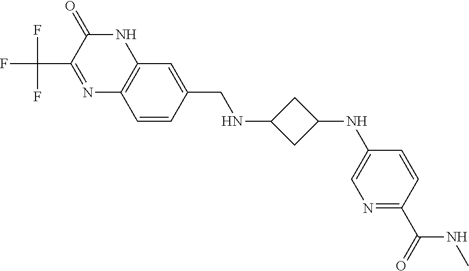 [SMILES: CNC(=O)c1ccc(NC2CC(NCc3ccc4nc(C(F)(F)F)c(=O)[nH]c4c3)C2)cn1] |
| 120 | N-methyl-5-(6-((3-oxo-2-(trifluoromethyl)-3,4- dihydroquinoxalin-6-yl)methyl)-2,6- diazaspiro[3.3]heptan-2-yl)picolinamide | 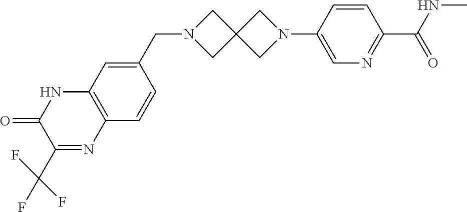 [SMILES: CNC(=O)c1ccc(N2CC3(CN(Cc4ccc5nc(C(F)(F)F)c(=O)[nH]c5c4)C3)C2)cn1] |
| 121 | N-methyl-5-((3-(((3-oxo-2-(2,2,2- trifluoroethyl)-3,4-dihydroquinoxalin-6- yl)methyl)amino)cyclobutyl)amino) picolinamide | 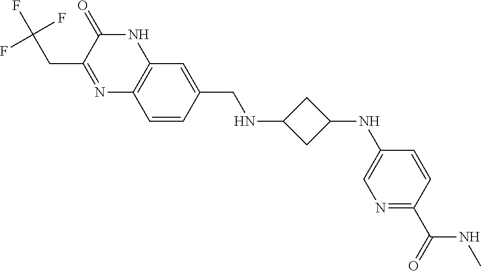 [SMILES: CNC(=O)c1ccc(NC2CC(NCc3ccc4nc(CC(F)(F)F)c(=O)[nH]c4c3)C2)cn1] |
| 122 | N-methyl-5-(6-((3-oxo-2-(2,2,2-trifluoroethyl)- 3,4-dihydroquinoxalin-6-yl)methyl)-2,6- diazaspiro[3.3]heptan-2-yl)picolinamide | 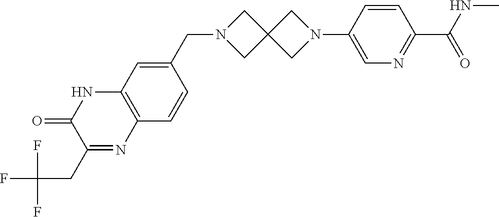 [SMILES: CNC(=O)c1ccc(N2CC3(CN(Cc4ccc5nc(CC(F)(F)F)c(=O)[nH]c5c4)C3)C2)cn1] |
| 123 | 7-ethyl-N-(3-((6-(methylcarbamoyl)pyridin-3- yl)amino)cyclobutyl)-6-oxo-5,6-dihydro-1,5- naphthyridine-3-carboxamide | 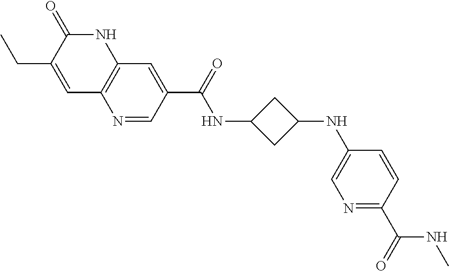 [SMILES: CCc1cc2ncc(C(=O)NC3CC(Nc4ccc(C(=O)NC)nc4)C3)cc2[nH]c1=O] |
| 124 | 5-(6-(7-ethyl-6-oxo-5,6-dihydro-1,5- naphthyridine-3-carbonyl)-2,6- diazaspiro[3.3]heptan-2-yl)-N- methylpicolinamide | 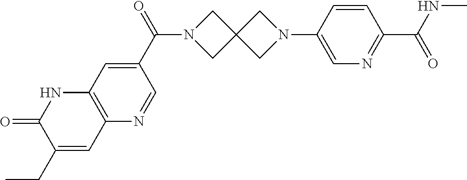 [SMILES: CCc1cc2ncc(C(=O)N3CC4(C3)CN(c3ccc(C(=O)NC)nc3)C4)cc2[nH]c1=O] |
| 125 | 5-((3-((1-(7-ethyl-6-oxo-5,6-dihydro-1,5- naphthyridin-3- yl)cyclopropyl)amino)cyclobutyl)amino)-N- methylpicolinamide | 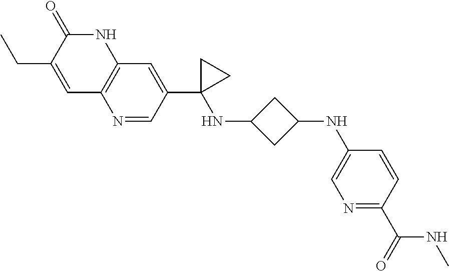 [SMILES: CCc1cc2ncc(C3(NC4CC(Nc5ccc(C(=O)NC)nc5)C4)CC3)cc2[nH]c1=O] |
| 126 | 5-(6-(1-(7-ethyl-6-oxo-5,6-dihydro-1,5- naphthyridin-3-yl)cyclopropyl)-2,6- diazaspiro[3.3]heptan-2-yl)-N- methylpicolinamide | 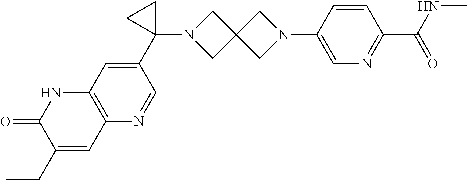 [SMILES: CCc1cc2ncc(C3(N4CC5(CN(c6ccc(C(=O)NC)nc6)C5)C4)CC3)cc2[nH]c1=O] |
| 127 | 5-((3-((2-(7-ethyl-6-oxo-5,6-dihydro-1,5- naphthyridin-3-yl)propan-2- yl)amino)cyclobutyl)amino)-N- methylpicolinamide | 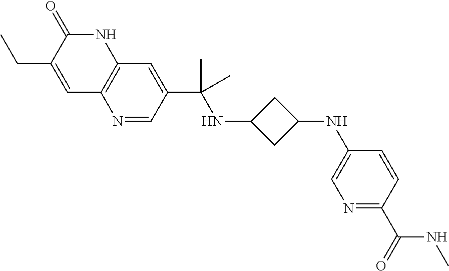 [SMILES: CCc1cc2ncc(C(C)(C)NC3CC(Nc4ccc(C(=O)NC)nc4)C3)cc2[nH]c1=O] |
| 128 | 5-(6-(2-(7-ethyl-6-oxo-5,6-dihydro-1,5- naphthyridin-3-yl)propan-2-yl)-2,6- diazaspiro[3.3]heptan-2-yl)-N- methylpicolinamide | 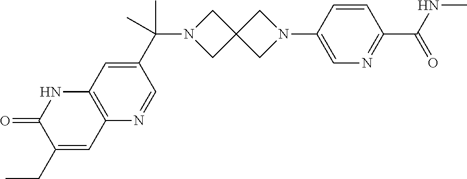 [SMILES: CCc1cc2ncc(C(C)(C)N3CC4(CN(c5ccc(C(=O)NC)nc5)C4)C3)cc2[nH]c1=O] |
| 129 | 5-(4-((7-ethyl-6-oxo-5,6-dihydro-1,5- naphthyridin-3-yl)amino)piperidin-1-yl)-N- methylpicolinamide | 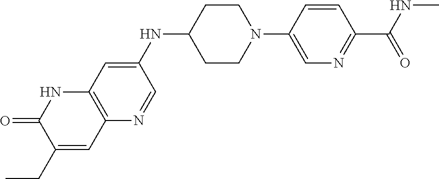 [SMILES: CCc1cc2ncc(NC3CCN(c4ccc(C(=O)NC)nc4)CC3)cc2[nH]c1=O] |
| 130 | 5-(4-((7-ethyl-6-oxo-5,6-dihydro-1,5- naphthyridin-3-yl)amino)piperidin-1-yl)-6- fluoro-N-methylpicolinamide | 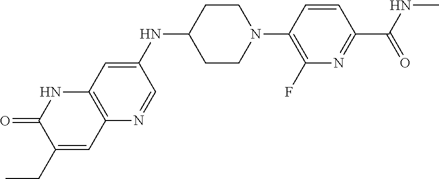 [SMILES: CCc1cc2ncc(NC3CCN(c4ccc(C(=O)NC)nc4F)CC3)cc2[nH]c1=O] |
| 131 | 5-(6-((7-ethyl-6-oxo-5,6-dihydro-1,5- naphthyridin-3-yl)methyl)-3,6- diazabicyclo[3.1.1]heptan-3-yl)-N,6- dimethylpicolinamide | 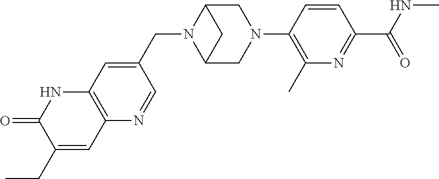 [SMILES: CCc1cc2ncc(CN3C4CC3CN(c3ccc(C(=O)NC)nc3C)C4)cc2[nH]c1=O] |
| 132 | 5-(3-((7-ethyl-6-oxo-5,6-dihydro-1,5- naphthyridin-3-yl)methyl)-3,6- diazabicyclo[3.1.1]heptan-6-yl)-N,6- dimethylpicolinamide | 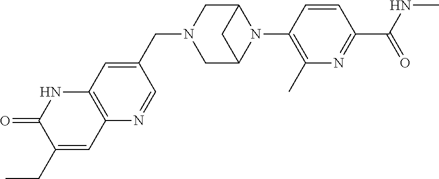 [SMILES: CCc1cc2ncc(CN3CC4CC(C3)N4c3ccc(C(=O)NC)nc3C)cc2[nH]c1=O] |
| 133 | 5-(8-((7-ethyl-6-oxo-5,6-dihydro-1,5- naphthyridin-3-yl)methyl)-3,8- diazabicyclo[3.2.1]octan-3-yl)-N,6- dimethylpicolinamide | 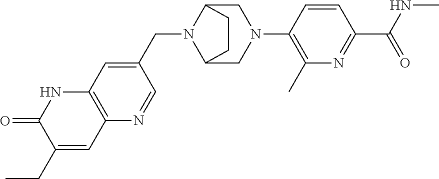 [SMILES: CCc1cc2ncc(CN3C4CCC3CN(c3ccc(C(=O)NC)nc3C)C4)cc2[nH]c1=O] |
| 134 | 5-(5-((7-ethyl-6-oxo-5,6-dihydro-1,5- naphthyridin-3-yl)methyl)-2,5- diazabicyclo[2.2.2]octan-2-yl)-N,6- dimethylpicolinamide | 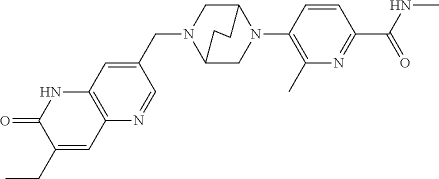 [SMILES: CCc1cc2ncc(CN3CC4CCC3CN4c3ccc(C(=O)NC)nc3C)cc2[nH]c1=O] |
| 135 | 5-(5-((7-ethyl-6-oxo-5,6-dihydro-1,5- naphthyridin-3-yl)methyl)-2,5- diazabicyclo[2.2.1]heptan-2-yl)-N,6- dimethylpicolinamide | 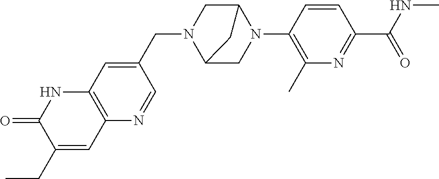 [SMILES: CCc1cc2ncc(CN3CC4CC3CN4c3ccc(C(=O)NC)nc3C)cc2[nH]c1=O] |
| 136 | 5-(4-((7-ethyl-6-oxo-5,6-dihydro-1,5- naphthyridin-3-yl)methyl)-1,4-diazepan-1-yl)- N,6-dimethylpicolinamide | 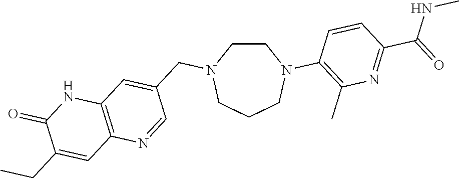 [SMILES: CCc1cc2ncc(CN3CCCN(c4ccc(C(=O)NC)nc4C)CC3)cc2[nH]c1=O] |
| 137 | 5-(5-((7-ethyl-6-oxo-5,6-dihydro-1,5- naphthyridin-3-yl)methyl)hexahydropyrrolo[3,4- c]pyrrol-2(1H)-yl)-N,6-dimethylpicolinamide | 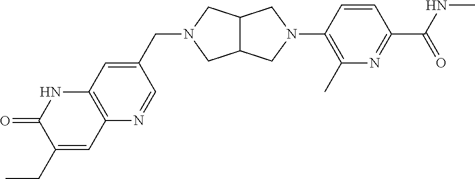 [SMILES: CCc1cc2ncc(CN3CC4CN(c5ccc(C(=O)NC)nc5C)CC4C3)cc2[nH]c1=O] |
| 138 | 5-(3-((7-ethyl-6-oxo-5,6-dihydro-1,5- naphthyridin-3-yl)methyl)-3,8- diazabicyclo[3.2.1]octan-8-yl)-N,6- dimethylpicolinamide | 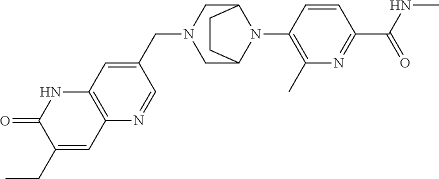 [SMILES: CCc1cc2ncc(CN3CC4CCC(C3)N4c3ccc(C(=O)NC)nc3C)cc2[nH]c1=O] |
| 139 | 5-(6-((7-ethyl-6-oxo-5,6-dihydro-1,5- naphthyridin-3-yl)methyl)-2,6- diazaspiro[3.3]heptan-2-yl)-N,6- dimethylpicolinamide | 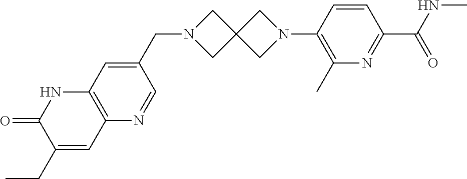 [SMILES: CCc1cc2ncc(CN3CC4(C3)CN(c3ccc(C(=O)NC)nc3C)C4)cc2[nH]c1=O] |
| 140 | 5-(4-((7-ethyl-6-oxo-5,6-dihydro-1,5- naphthyridin-3-yl)methyl)-2-oxo-1,4-diazepan- 1-yl)-N,6-dimethylpicolinamide | 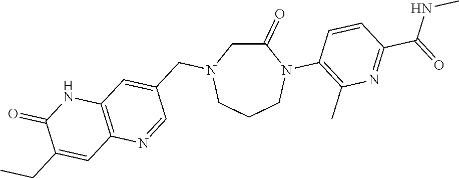 [SMILES: CCc1cc2ncc(CN3CCCN(c4ccc(C(=O)NC)nc4C)C(=O)C3)cc2[nH]c1=O] |
| 141 | 5-(4-((7-ethyl-6-oxo-5,6-dihydro-1,5- naphthyridin-3-yl)methyl)-7-oxo-1,4-diazepan- 1-yl)-N,6-dimethylpicolinamide | 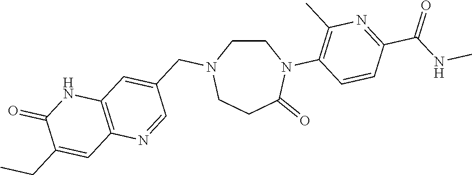 [SMILES: CCc1cc2ncc(CN3CCC(=O)N(c4ccc(C(=O)NC)nc4C)CC3)cc2[nH]c1=O] |
| 142 | 5-(5-((7-ethyl-6-oxo-5,6-dihydro-1,5- naphthyridin-3-yl)methyl)-2-oxa-5,8- diazaspiro[3.5]nonan-8-yl)-N,6- dimethylpicolinamide | 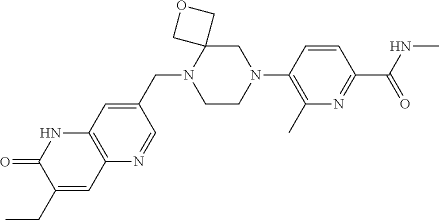 [SMILES: CCc1cc2ncc(CN3CCN(c4ccc(C(=O)NC)nc4C)CC34COC4)cc2[nH]c1=O] |
| 143 | 5-(4-((7-ethyl-6-oxo-5,6-dihydro-1,5- naphthyridin-3-yl)methyl)-4,7- diazaspiro[2.5]octan-7-yl)-N,6- dimethylpicolinamide | 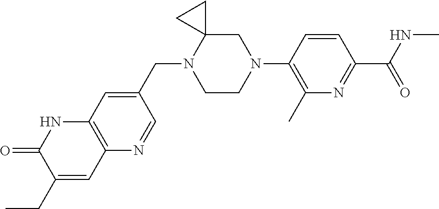 [SMILES: CCc1cc2ncc(CN3CCN(c4ccc(C(=O)NC)nc4C)CC34CC4)cc2[nH]c1=O] |
| 144 | 5-(5-((7-ethyl-6-oxo-5,6-dihydro-1,5- naphthyridin-3-yl)methyl)-5,8- diazaspiro[3.5]nonan-8-yl)-N,6- dimethylpicolinamide | 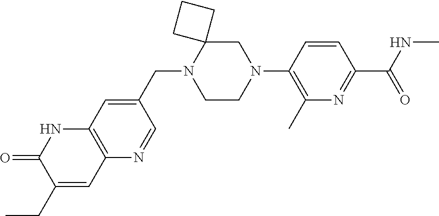 [SMILES: CCc1cc2ncc(CN3CCN(c4ccc(C(=O)NC)nc4C)CC34CCC4)cc2[nH]c1=O] |
| 145 | 5-(6-((7-ethyl-6-oxo-5,6-dihydro-1,5- naphthyridin-3-yl)methyl)-2-oxa-6,9- diazaspiro[4.5]decan-9-yl)-N,6- dimethylpicolinamide | 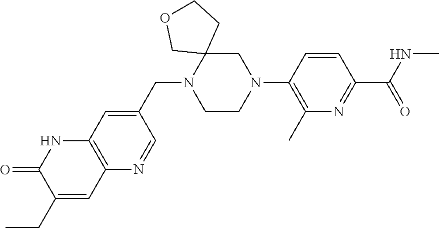 [SMILES: CCc1cc2ncc(CN3CCN(c4ccc(C(=O)NC)nc4C)CC34CCOC4)cc2[nH]c1=O] |
| 146 | 5-(8-((7-ethyl-6-oxo-5,6-dihydro-1,5- naphthyridin-3-yl)methyl)-2-oxa-5,8- diazaspiro[3.5]nonan-5-yl)-N,6- dimethylpicolinamide | 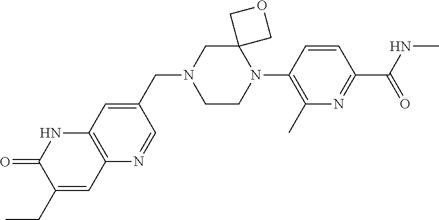 [SMILES: CCc1cc2ncc(CN3CCN(c4ccc(C(=O)NC)nc4C)C4(COC4)C3)cc2[nH]c1=O] |
| 147 | 5-(7-((7-ethyl-6-oxo-5,6-dihydro-1,5- naphthyridin-3-yl)methyl)-4,7- diazaspiro[2.5]octan-4-yl)-N,6- dimethylpicolinamide | 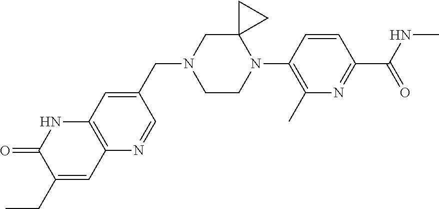 [SMILES: CCc1cc2ncc(CN3CCN(c4ccc(C(=O)NC)nc4C)C4(CC4)C3)cc2[nH]c1=O] |
| 148 | 5-(8-((7-ethyl-6-oxo-5,6-dihydro-1,5- naphthyridin-3-yl)methyl)-5,8- diazaspiro[3.5]nonan-5-yl)-N,6- dimethylpicolinamide | 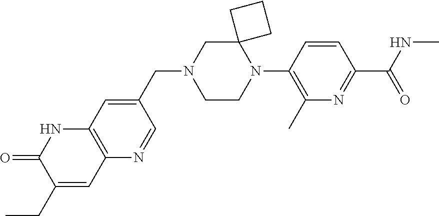 [SMILES: CCc1cc2ncc(CN3CCN(c4ccc(C(=O)NC)nc4C)C4(CCC4)C3)cc2[nH]c1=O] |
| 149 | 5-(9-((7-ethyl-6-oxo-5,6-dihydro-1,5- naphthyridin-3-yl)methyl)-2-oxa-6,9- diazaspiro[4.5]decan-6-yl)-N,6- dimethylpicolinamide | 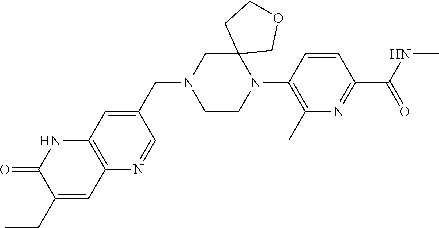 [SMILES: CCc1cc2ncc(CN3CCN(c4ccc(C(=O)NC)nc4C)C4(CCOC4)C3)cc2[nH]c1=O] |
| 150 | 5-((1-((7-ethyl-6-oxo-5,6-dihydro-1,5- naphthyridin-3-yl)methyl)piperidin-4-yl)amino)- N,6-dimethylpicolinamide | 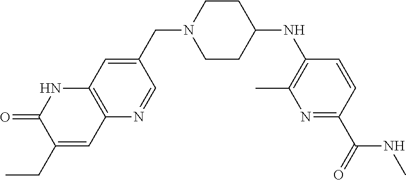 [SMILES: CCc1cc2ncc(CN3CCC(Nc4ccc(C(=O)NC)nc4C)CC3)cc2[nH]c1=O] |
| 151 | 5-((2-((7-ethyl-6-oxo-5,6-dihydro-1,5- naphthyridin-3- yl)methyl)octahydrocyclopenta[c]pyrrol-5- yl)amino)-N,6-dimethylpicolinamide | 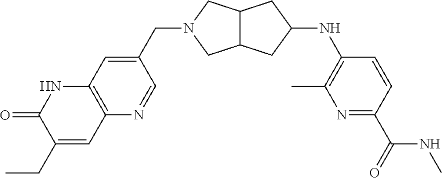 [SMILES: CCc1cc2ncc(CN3CC4CC(Nc5ccc(C(=O)NC)nc5C)CC4C3)cc2[nH]c1=O] |
| 152 | 5-((3-((7-ethyl-6-oxo-5,6-dihydro-1,5- naphthyridin-3-yl)methyl)-3- azabicyclo[3.1.0]hexan-6-yl)amino)-N,6- dimethylpicolinamide | 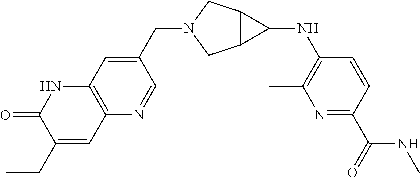 [SMILES: CCc1cc2ncc(CN3CC4C(C3)C4Nc3ccc(C(=O)NC)nc3C)cc2[nH]c1=O] |
| 153 | 5-((1-((7-ethyl-6-oxo-5,6-dihydro-1,5- naphthyridin-3-yl)methyl)azetidin-3-yl)amino)- N,6-dimethylpicolinamide | 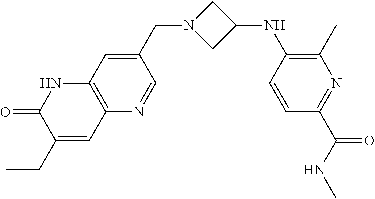 [SMILES: CCc1cc2ncc(CN3CC(Nc4ccc(C(=O)NC)nc4C)C3)cc2[nH]c1=O] |
| 154 | 5-(((1-((7-ethyl-6-oxo-5,6-dihydro-1,5- naphthyridin-3-yl)methyl)azetidin-3- yl)methyl)amino)-N,6-dimethylpicolinamide | 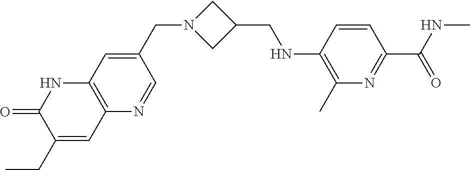 [SMILES: CCc1cc2ncc(CN3CC(CNc4ccc(C(=O)NC)nc4C)C3)cc2[nH]c1=O] |
| 155 | 5-((1-((7-ethyl-6-oxo-5,6-dihydro-1,5- naphthyridin-3-yl)methyl)azetidin-3-yl)oxy)- N,6-dimethylpicolinamide | 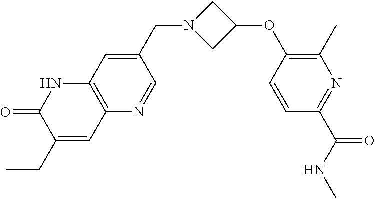 [SMILES: CCc1cc2ncc(CN3CC(Oc4ccc(C(=O)NC)nc4C)C3)cc2[nH]c1=O] |
| 156 | 5-((1-((7-ethyl-6-oxo-5,6-dihydro-1,5- naphthyridin-3-yl)methyl)azetidin-3- yl)methoxy)-N,6-dimethylpicolinamide | 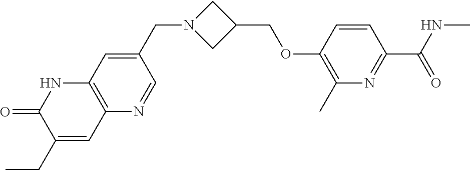 [SMILES: CCc1cc2ncc(CN3CC(COc4ccc(C(=O)NC)nc4C)C3)cc2[nH]c1=O] |
| 157 | 5-(4-((7-ethyl-6-oxo-5,6-dihydro-1,5- naphthyridin-3-yl)methyl)-4-hydroxypiperidin- 1-yl)-N,6-dimethylpicolinamide | 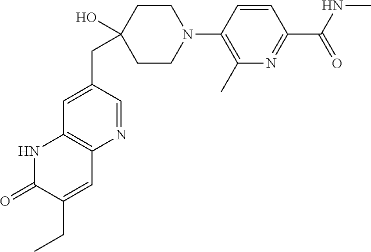 [SMILES: CCc1cc2ncc(CC3(O)CCN(c4ccc(C(=O)NC)nc4C)CC3)cc2[nH]c1=O] |
| 158 | 5-(4-((7-ethyl-6-oxo-5,6-dihydro-1,5- naphthyridin-3-yl)methyl)-4-fluoropiperidin-1- yl)-N,6-dimethylpicolinamide | 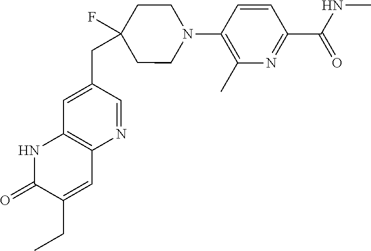 [SMILES: CCc1cc2ncc(CC3(F)CCN(c4ccc(C(=O)NC)nc4C)CC3)cc2[nH]c1=O] |
| 159 | 5-((3-(((7-ethyl-6-oxo-5,6-dihydro-1,5- naphthyridin-3- yl)methyl)amino)cyclobutyl)amino)-N,6- dimethylpicolinamide | 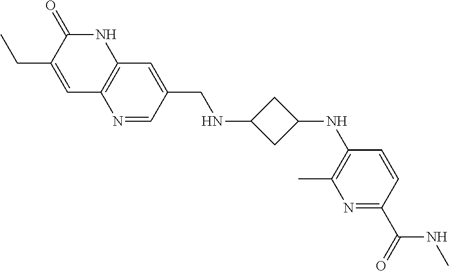 [SMILES: CCc1cc2ncc(CNC3CC(Nc4ccc(C(=O)NC)nc4C)C3)cc2[nH]c1=O] |
| 160 | 5-((3-(((7-ethyl-6-oxo-5,6-dihydro-1,5- naphthyridin-3- yl)methyl)amino)bicyclo[1.1.1]pentan-1- yl)amino)-N,6-dimethylpicolinamide | 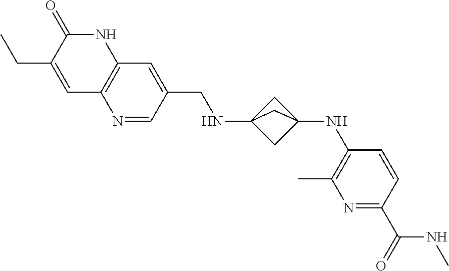 [SMILES: CCc1cc2ncc(CNC34CC(Nc5ccc(C(=O)NC)nc5C)(C3)C4)cc2[nH]c1=O] |
| 161 | 5-((4-(((7-ethyl-6-oxo-5,6-dihydro-1,5- naphthyridin-3- yl)methyl)amino)cyclohexyl)amino)-N,6- dimethylpicolinamide | 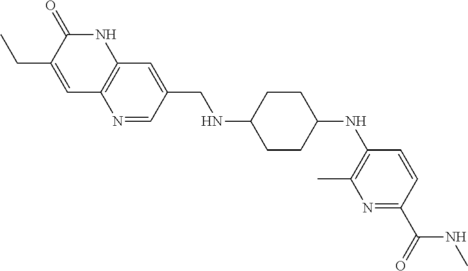 [SMILES: CCc1cc2ncc(CNC3CCC(Nc4ccc(C(=O)NC)nc4C)CC3)cc2[nH]c1=O] |
| 162 | 5-((4-(((7-ethyl-6-oxo-5,6-dihydro-1,5- naphthyridin-3- yl)methyl)amino)bicyclo[2.2.2]octan-1- yl)amino)-N,6-dimethylpicolinamide | 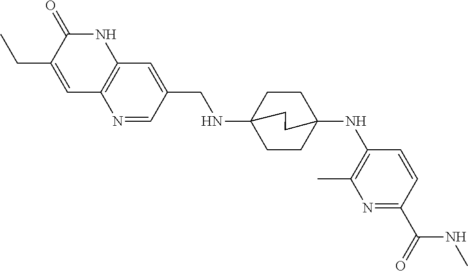 [SMILES: CCc1cc2ncc(CNC34CCC(Nc5ccc(C(=O)NC)nc5C)(CC3)CC4)cc2[nH]c1=O] |
| 163 | 5-((4-(((7-ethyl-6-oxo-5,6-dihydro-1,5- naphthyridin-3- yl)methyl)amino)bicyclo[2.2.1]heptan-1- yl)amino)-N,6-dimethylpicolinamide | 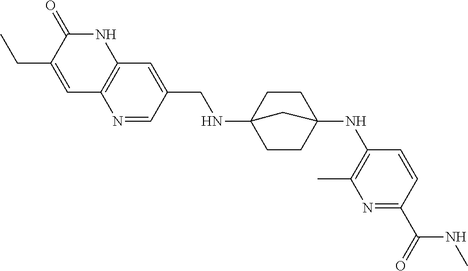 [SMILES: CCc1cc2ncc(CNC34CCC(Nc5ccc(C(=O)NC)nc5C)(CC3)C4)cc2[nH]c1=O] |
| 164 | 5-(((2-(((7-ethyl-6-oxo-5,6-dihydro-1,5- naphthyridin-3- yl)methyl)amino)cyclopropyl)methyl)amino)- N,6-dimethylpicolinamide | 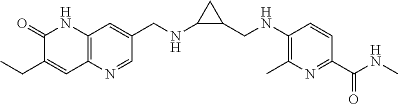 [SMILES: CCc1cc2ncc(CNC3CC3CNc3ccc(C(=O)NC)nc3C)cc2[nH]c1=O] |
| 165 | 5-(4-(((7-ethyl-6-oxo-5,6-dihydro-1,5- naphthyridin-3-yl)methyl)amino)piperidin-1-yl)- N,6-dimethylpicolinamide | 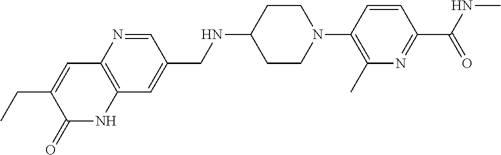 [SMILES: CCc1cc2ncc(CNC3CCN(c4ccc(C(=O)NC)nc4C)CC3)cc2[nH]c1=O] |
| 166 | 5-(6-((2-ethyl-3-oxo-3,4-dihydroquinoxalin-6- yl)methyl)-2,6-diazaspiro[3.3]heptan-2-yl)-N,6- dimethylpicolinamide | 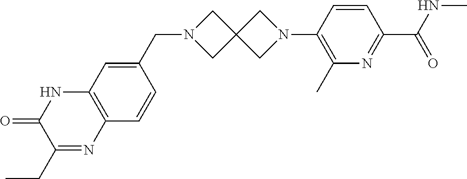 [SMILES: CCc1nc2ccc(CN3CC4(C3)CN(c3ccc(C(=O)NC)nc3C)C4)cc2[nH]c1=O] |
| 167 | 5-(6-((3-ethyl-2-oxo-1,2-dihydropyrido[2,3- b]pyrazin-7-yl)methyl)-2,6- diazaspiro[3.3]heptan-2-yl)-N,6- dimethylpicolinamide | 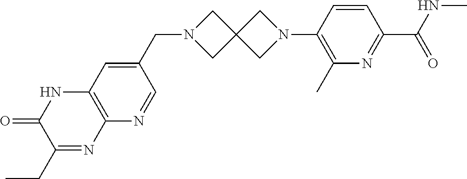 [SMILES: CCc1nc2ncc(CN3CC4(C3)CN(c3ccc(C(=O)NC)nc3C)C4)cc2[nH]c1=O] |
| 168 | 5-(6-((3-ethyl-2-oxo-1,2-dihydropyrido[3,4- b]pyrazin-7-yl)methyl)-2,6- diazaspiro[3.3]heptan-2-yl)-N,6- dimethylpicolinamide | 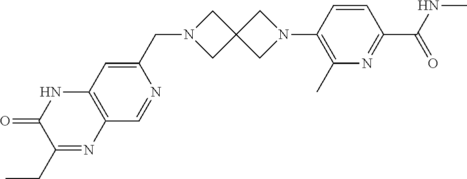 [SMILES: CCc1nc2cnc(CN3CC4(C3)CN(c3ccc(C(=O)NC)nc3C)C4)cc2[nH]c1=O] |
| 169 | 5-(6-((3-ethyl-2-oxo-1,2-dihydroquinolin-7- yl)methyl)-2,6-diazaspiro[3.3]heptan-2-yl)-N,6- dimethylpicolinamide | 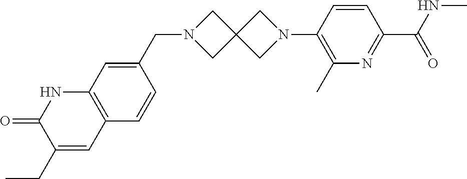 [SMILES: CCc1cc2ccc(CN3CC4(C3)CN(c3ccc(C(=O)NC)nc3C)C4)cc2[nH]c1=O] |
| 171 | 5-(6-((3-ethyl-2-oxo-1,2-dihydro-1,6- naphthyridin-7-yl)methyl)-2,6- diazaspiro[3.3]heptan-2-yl)-N,6- dimethylpicolinamide | 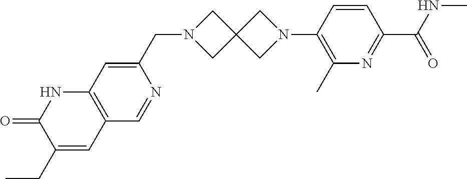 [SMILES: CCc1cc2cnc(CN3CC4(C3)CN(c3ccc(C(=O)NC)nc3C)C4)cc2[nH]c1=O] |
| 172 | 7-((6-(2,4-difluorophenyl)-2,6- diazaspiro[3.3]heptan-2-yl)methyl)-3- ethylquinoxalin-2(1H)-one | 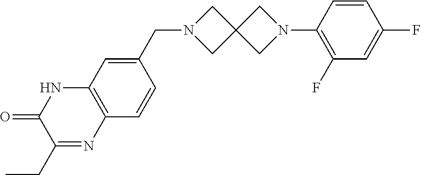 [SMILES: CCc1nc2ccc(CN3CC4(C3)CN(c3ccc(F)cc3F)C4)cc2[nH]c1=O] |
| 173 | 7-((6-(2,4-difluorophenyl)-2,6- diazaspiro[3.3]heptan-2-yl)methyl)-3- ethylpyrido[2,3-b]pyrazin-2(1H)-one | 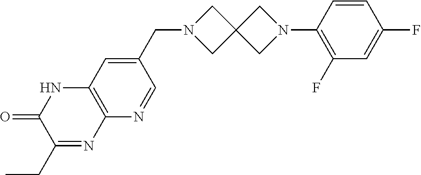 [SMILES: CCc1nc2ncc(CN3CC4(C3)CN(c3ccc(F)cc3F)C4)cc2[nH]c1=O] |
| 174 | 7-((6-(2,4-difluorophenyl)-2,6- diazaspiro[3.3]heptan-2-yl)methyl)-3- ethylpyrido[3,4-b]pyrazin-2(1H)-one | 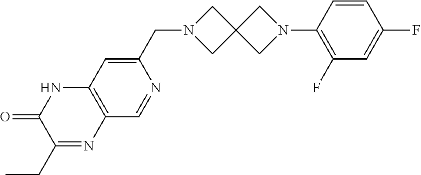 [SMILES: CCc1nc2cnc(CN3CC4(C3)CN(c3ccc(F)cc3F)C4)cc2[nH]c1=O] |
| 175 | 7-((6-(2,4-difluorophenyl)-2,6- diazaspiro[3.3]heptan-2-yl)methyl)-3- ethylquinolin-2(1H)-one | 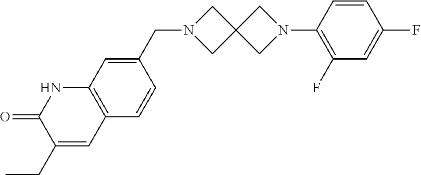 [SMILES: CCc1cc2ccc(CN3CC4(C3)CN(c3ccc(F)cc3F)C4)cc2[nH]c1=O] |
| 176 | 7-((6-(2,4-difluorophenyl)-2,6- diazaspiro[3.3]heptan-2-yl)methyl)-3-ethyl-1,5- naphthyridin-2(1H)-one | 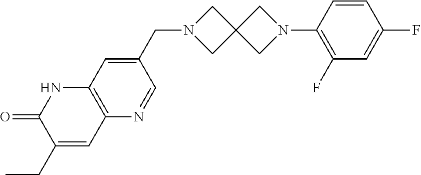 [SMILES: CCc1cc2ncc(CN3CC4(C3)CN(c3ccc(F)cc3F)C4)cc2[nH]c1=O] |
| 177 | 7-((6-(2,4-difluorophenyl)-2,6- diazaspiro[3.3]heptan-2-yl)methyl)-3-ethyl-1,6- naphthyridin-2(1H)-one | 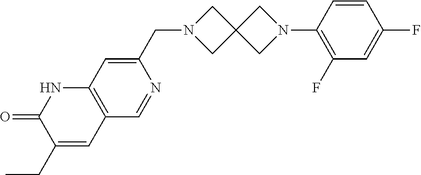 [SMILES: CCc1cc2cnc(CN3CC4(C3)CN(c3ccc(F)cc3F)C4)cc2[nH]c1=O] |
| 178 | 5-(6-((7-ethyl-6-oxo-5,6-dihydro-1,5- naphthyridin-3-yl)methyl)-2,6- diazaspiro[3.3]heptane-2-carbonyl)-N- methylpicolinamide | 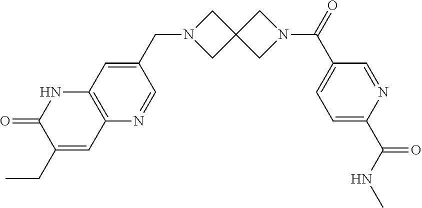 [SMILES: CCc1cc2ncc(CN3CC4(C3)CN(C(=O)c3ccc(C(=O)NC)nc3)C4)cc2[nH]c1=O] |
| 179 | 5-(6-((7-ethyl-6-oxo-5,6-dihydro-1,5- naphthyridin-3-yl)methyl)-2,6- diazaspiro[3.3]heptane-2-carbonyl)-6-fluoro-N- methylpicolinamide | 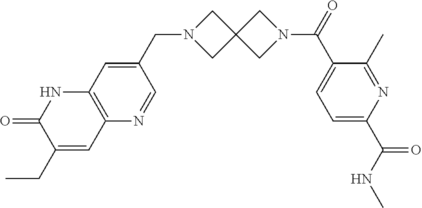 [SMILES: CCc1cc2ncc(CN3CC4(C3)CN(C(=O)c3ccc(C(=O)NC)nc3C)C4)cc2[nH]c1=O] |
| 180 | 5-(6-((7-ethyl-6-oxo-5,6-dihydro-1,5- naphthyridin-3-yl)methyl)-2,6- diazaspiro[3.3]heptane-2-carbonyl)-N,6- dimethylpicolinamide | 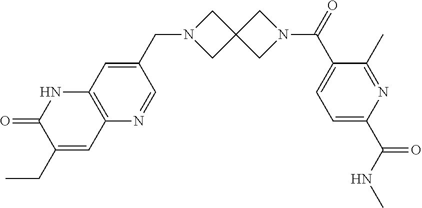 [SMILES: CCc1cc2ncc(CN3CC4(C3)CN(C(=O)c3ccc(C(=O)NC)nc3C)C4)cc2[nH]c1=O] |
| 181 | 7-((6-(2,4-difluorobenzoyl)-2,6- diazaspiro[3.3]heptan-2-yl)methyl)-3-ethyl-1,5- naphthyridin-2(1H)-one | 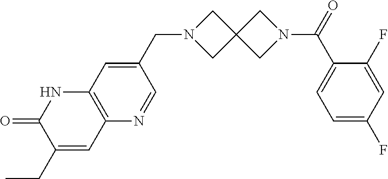 [SMILES: CCc1cc2ncc(CN3CC4(C3)CN(C(=O)c3ccc(F)cc3F)C4)cc2[nH]c1=O] |

In some embodiments, the compound is a pharmaceutically acceptable salt of any one of the compounds described in Table 1.

the neutral compounds of Formula (I) may be converted to the corresponding pharmaceutically acceptable salts of the compounds using techniques in the art (e.g., by saponification of an ester to the carboxylic acid salt, or by hydrolyzing an amide to form a corresponding carboxylic acid and then converting the carboxylic acid to a carboxylic acid salt).

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognised techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. In some embodiments, the molecular weight of the compound will be less than 900, for example less than 800, or less than 750, or less than 700, or less than 650 daltons. For example, the molecular weight is less than 600 and, for example, is 550 daltons or less.

A suitable pharmaceutically acceptable salt of a compound of the disclosure is, for example, an acid-addition salt of a compound of the disclosure, which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof, while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess (1) PARP and/or (2) PARP-1 inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess (1) PARP and/or (2) PARP-1 inhibitory activity.

It is also to be understood that certain compounds of any one of the Formulae disclosed herein may exhibit polymorphism, and that the disclosure encompasses all such forms, or mixtures thereof, which possess (1) PARP and/or (2) PARP-1 inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

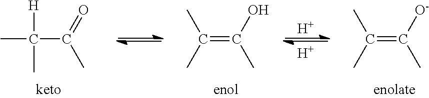

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesised by any one of the processes defined herein, the processes may then further comprise the additional steps of: (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula (I) can be isolated and purified using techniques well known in the art.

For example, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes 1-8 herein.

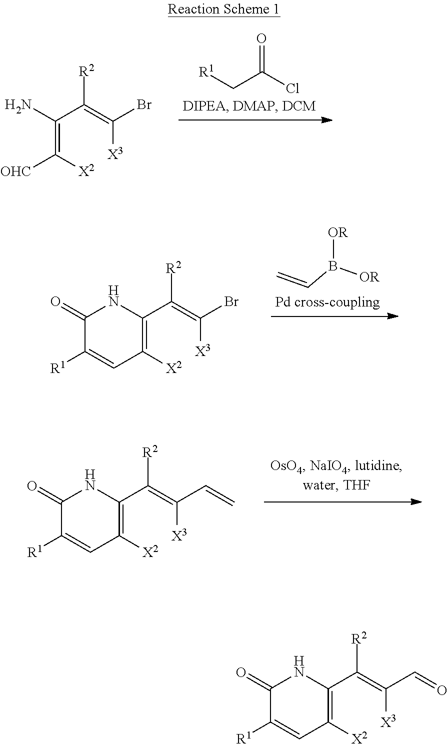

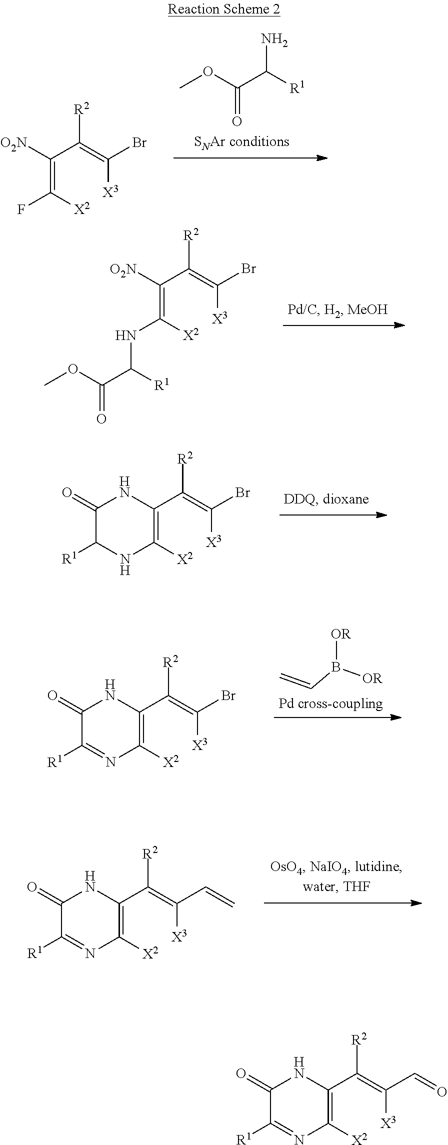

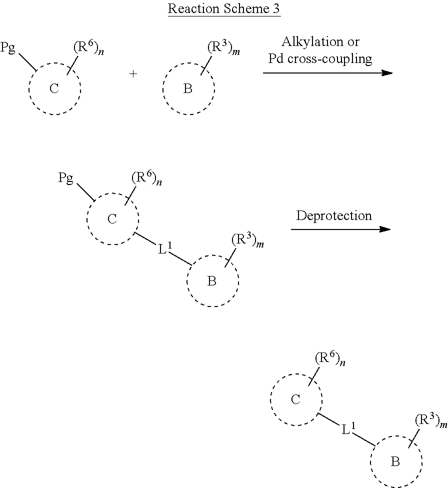

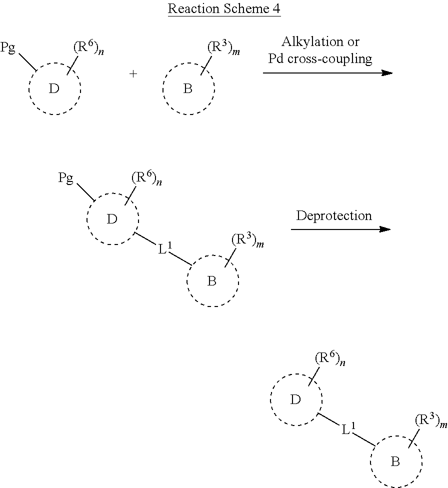

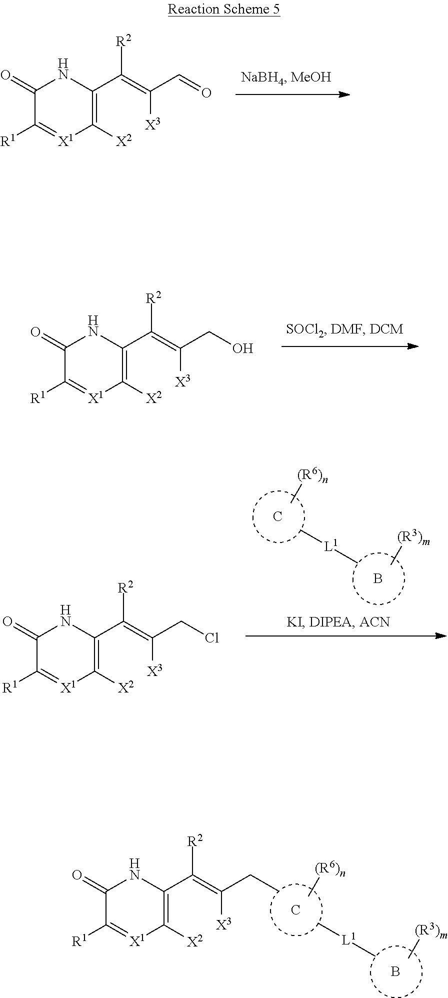

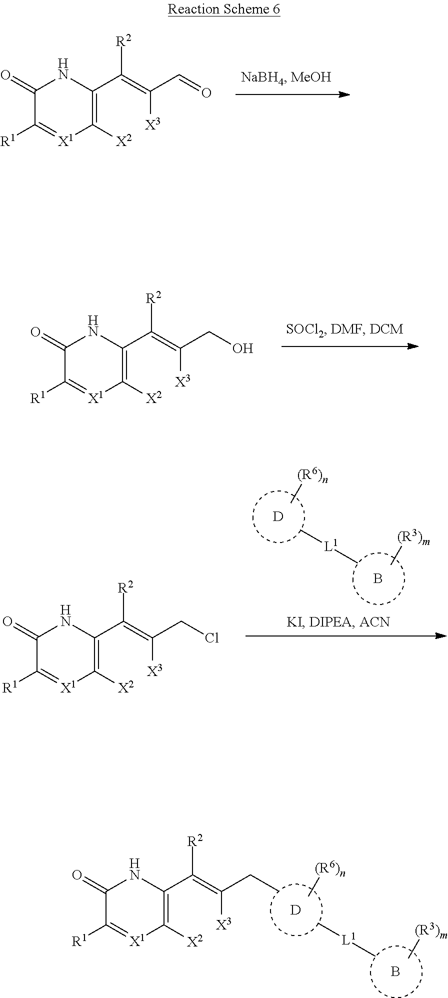

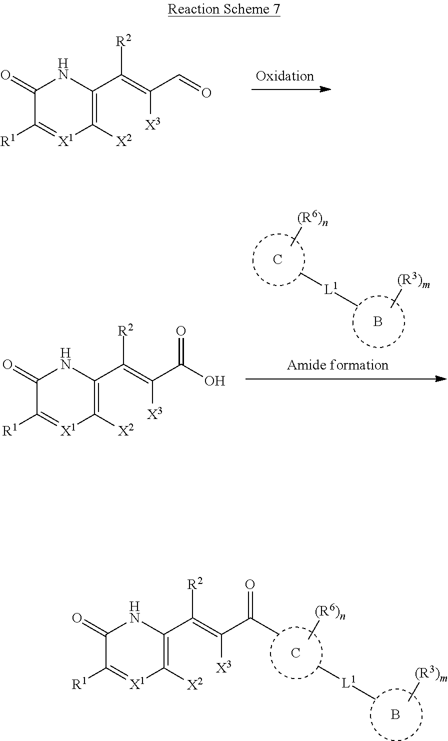

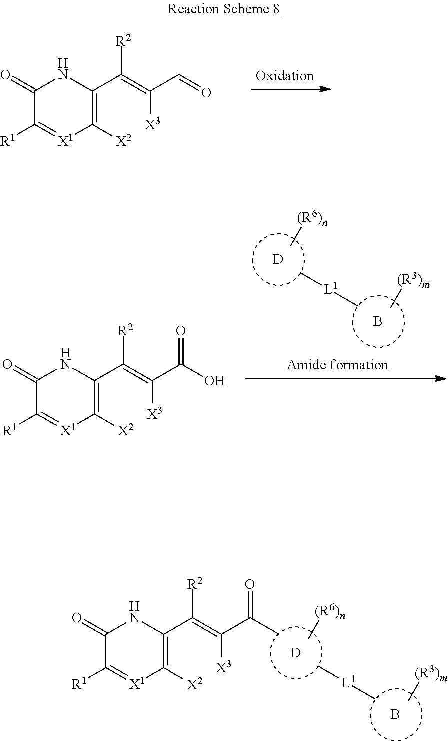

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein. In some embodiments, the biological assay measures PARP-1 binding by way of fluorescence polarization measurements. In some embodiments, binding of a test compound to recombinant human PARP-1 reduces the amount of binding of a fluorescent probe, and consequently reduces the detected level of fluorescence polarization. In some test compound stock solutions are prepared in a solvent, e.g., DMSO, and are serially diluted into a range of concentrations, e.g., 15 concentrations, 14 concentrations, 13 concentrations, 12 concentrations, 11 concentrations, 10 concentrations, 9 concentrations, 8 concentrations, 7 concentrations, 6 concentrations, or 5 concentrations, by a dilution factor, e.g., a 10-fold dilution factor, a 9-fold dilution factor, an 8-fold dilution factor, a 7-fold dilution factor, a 6-fold dilution factor, a 5-fold dilution factor, a 4-fold dilution factor, a 3-fold dilution factor, or a 2-fold dilution factor. In some embodiments, the serial dilutions, e.g., 100 nL of serial dilution, 90 nL of serial dilution, 80 nL of serial dilution, 70 nL of serial dilution, 60 nL of serial dilution, 50 nL of serial dilution, 30 nL of serial dilution, 20 nL of serial dilution, or 10 nL of serial dilution, are added to a multi-welled plate, e.g., a 1536-well plate, a 384-well plate, or a 96-well plate. In some embodiments, PARP-1, e.g., GST-tagged PARP-1, is diluted in assay buffer, e.g., 50 μL of assay buffer, 40 μL of assay buffer, e.g., 30 μL of assay buffer, 20 μL of assay buffer, 10 μL of assay buffer, or 5 μL of assay buffer, and is added to the plate. In some embodiments, the plate is centrifuged, e.g., at 1000 rpm for 1 min, and incubated, e.g., incubated for 30 minutes at room temperature (RT). In some embodiments, a fluorescent probe, e.g., 10 μL of 6 nM PARPi-FL (TOCRIS, Cat #6461), is diluted in an assay buffer and is added to the plate. In some embodiments, the final concentrations of PARP-1 and fluorescent probe are 20 nM and 3 nM, respectively, in a total volume of 20 μL. In some embodiments, the plate is centrifuged, e.g., at 1000 rpm for 1 minute, and the assay plate is incubated, e.g., for 4 h at RT. In some embodiments, the samples in each well are read using a plate reader, e.g., an Envision instrument using excitation=480 nm, and emission wavelength=FITC FP-P pol 535 nm & FITC FP-S pol 535 nm). In some embodiments, percent inhibition is calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. In some embodiments, binding IC₅₀ values are calculated using XLFit (equation 201: y=A+((B−A)/(1+((x/C)\hat{ }D))), where A=bottom, B=top, C=IC₅₀, and D=slope) with a floating top and bottom for curves.

In some embodiments, the biological assay measures PARP-2 binding by way of fluorescence polarization measurements. In some embodiments, binding of a test compound to recombinant human PARP-2 reduces the amount of binding of a fluorescent probe, and consequently reduces the detected level of fluorescence polarization. In some test compound stock solutions are prepared in a solvent, e.g., DMSO, and are serially diluted into a range of concentrations, e.g., 15 concentrations, 14 concentrations, 13 concentrations, 12 concentrations, 11 concentrations, 10 concentrations, 9 concentrations, 8 concentrations, 7 concentrations, 6 concentrations, or 5 concentrations, by a dilution factor, e.g., a 10-fold dilution factor, a 9-fold dilution factor, an 8-fold dilution factor, a 7-fold dilution factor, a 6-fold dilution factor, a 5-fold dilution factor, a 4-fold dilution factor, a 3-fold dilution factor, or a 2-fold dilution factor. In some embodiments, the serial dilutions, e.g., 100 nL of serial dilution, 90 nL of serial dilution, 80 nL of serial dilution, 70 nL of serial dilution, 60 nL of serial dilution, 50 nL of serial dilution, 30 nL of serial dilution, 20 nL of serial dilution, or 10 nL of serial dilution, are added to a multi-welled plate, e.g., a 1536-well plate, a 384-well plate, or a 96-well plate. In some embodiments, PARP-2, e.g., GST-tagged PARP-2, is diluted in assay buffer, e.g., 50 μL of assay buffer, 40 μL of assay buffer, e.g., 30 μL of assay buffer, 20 μL of assay buffer, 10 μL of assay buffer, or 5 μL of assay buffer, and is added to the plate. In some embodiments, the plate is centrifuged, e.g., at 1000 rpm for 1 min, and incubated, e.g., incubated for 30 minutes at room temperature (RT). In some embodiments, a fluorescent probe, e.g., 10 μL of 6 nM PARPi-FL (TOCRIS, Cat #6461), is diluted in an assay buffer and is added to the plate. In some embodiments, the final concentrations of PARP-2 and fluorescent probe are 20 nM and 3 nM, respectively, in a total volume of 20 μL. In some embodiments, the plate is centrifuged, e.g., at 1000 rpm for 1 minute, and the assay plate is incubated, e.g., for 4 h at RT. In some embodiments, the samples in each well are read using a plate reader, e.g., an Envision instrument using excitation=480 nm, and emission wavelength=FITC FP-P pol 535 nm & FITC FP-S pol 535 nm). In some embodiments, percent inhibition is calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. In some embodiments, binding IC₅₀ values are calculated using XLFit (equation 201: y=A+((B−A)/(1+((x/C)\hat{ }D))), where A=bottom, B=top, C=IC₅₀, and D=slope) with a floating top and bottom for curves.

In some embodiments, the biological assay measures the effects that compounds of the present disclosure may have on the viability of cells, e.g., the viability of cancer cells. In some embodiments, DLD-1 wild-type and DLD-1 BRCA2(−/−) colorectal adenocarcinoma cells, an isogenic pair of cell lines differing in the presence and absence, respectively, of both BRCA2 alleles, are used to measure the effect of the disclosed compounds on the viability of cancer cells.

In some embodiments, the cells are harvested during the logarithmic growth period, counted, and seeded at a specified count in the wells of a 96- or 384-well cell culture plate. In some embodiments, after seeding, the cells are incubated, e.g., at 37° C., 5% CO₂ overnight. In some embodiments, the cells are treated with compounds, e.g., with serially diluted test compounds at 8-11 concentrations within a desired concentration range, e.g., from 1 nM-10 μM, for the generation of dose-response curves. In some embodiments, the plate is further incubated, e.g., for another 3-7 days in a humidified incubator at 37° C. and 5% CO₂. In some embodiments, cell viability is assessed by luminescence measurement after addition of Cell Titer-Glo reagent (Promega, Madison, Wis.) according to the manufacturer's instructions. In some embodiments, cell viability IC₅₀ values are calculated using XLFit, equation 201: y=A+((B−A)/(1+((x/C)\hat{ }D))), where A=bottom, B=top, C=IC₅₀, and D=slope. In some embodiments, the effects of the test compounds on the viability of other cell lines such as MDA-MB-436, MDA-MB-231, SUM149PT, HCC1395, and UWB1.289 are determined in an analogous method.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy in an amount sufficient to treat or prevent (1) a PARP and/or (2) a PARP-1 related condition referred to herein, slow its progression and/or reduce the symptoms associated with the condition.

An effective amount of a compound of the present disclosure for use in therapy in an amount sufficient to treat (1) a PARP and/or (2) a PARP-1 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

Homologous recombination (HR) is a process critical to the repair of DNA double-strand breaks (DSBs) that, when defective, leads to the accumulation of DNA damage and genomic instability (Mekonnen N, et al. Frontiers in oncology. 2022:2747). Defective or deficient HR can result from mutation, loss of function, or inactivation of one or more genes involved in HR.

BRCA1 and BRCA2 are important tumor suppressor genes with a central role in the repair of DNA DSBs by HR. Deleterious mutations in BRCA1 and/or BRCA2 result in HR deficiency and are linked to increased risk of several cancers, most notably breast and ovarian cancer. Other genes involved in HR-mediated repair and whose mutation, loss of function, or inactivation can contribute to defects in HR include members of the FANC gene family, ATM, RAD51, PALB2, MRE11A, RAD50, NBS1, and EMSY.

Some sporadic cancers do not carry BRCA1 or BRCA2 mutations but display a BRCAness phenotype. Cancers with a BRCAness phenotype are cancers that share phenotypic characteristics of cancers that have germline BRCA1 or BRCA2 mutations, particularly HR deficiency (Turner N, et al. Nature reviews cancer. 2004 October;4(10):814-9). The HR deficiency may result from inactivation of tumor suppressor genes including HR repair genes by mutation, loss of heterozygosity, or promoter hypermethylation. Phenotypic characteristics of cancers with germline BRCA1 or BRCA2 mutations are generally associated with genomic instability and include both chromosomal and sub-chromosomal aberrations such as mutations, structural copy number changes, and/or structural rearrangements (Mekonnen N, et al., 2022).

PARP inhibition has been found to be synthetically lethal to cancers with mutations in BRCA1 and/or BRCA2, both preclinically and clinically (Lord C J, et al. Science. 2017 Mar. 17; 355(6330):1152-8). Cancers with a BRCAness phenotype are also thought to be particularly susceptible to PARP inhibition. PARP inhibitors are part of standard-of-care treatments for cancers with defects in HR repair of DNA. These cancers include HR-deficient breast, ovarian, prostate and pancreatic cancers.

In some aspects, the present disclosure provides a method of modulating PARP1 activity (e.g., in vitro or in vivo), comprising contacting a cell with an effective amount of a compound of the present disclosure or a pharmaceutically acceptable salt thereof.

In some aspects, the present disclosure provides a method of modulating PARP1 activity (e.g., in vitro or in vivo), comprising contacting a cell with a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated PARP1 activity. In some embodiments, the disease or disorder is a disease or disorder in which PARP1 activity is implicated.

In some embodiments, the disease or disorder is cancer.

In some embodiments, the disease or disorder is an HR-deficient cancer.

In some embodiments, the disease or disorder is a BRCA1- or BRCA2-mutated cancer.

In some aspects, the present disclosure provides a method of treating or preventing cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing HR-deficient cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing BRCA1- or BRCA2-mutated cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating HR-deficient cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating BRCA1- or BRCA2-mutated cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing HR-deficient cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing BRCA1- or BRCA2-mutated cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating HR-deficient cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating BRCA1- or BRCA2-mutated cancer in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating PARP1 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing cancer in a subject in need thereof.

In some aspects, the present disclosure provides a compound of the present disclosure or a pharmaceutically acceptable salt thereof for use in treating or preventing HR-deficient cancer in a subject in need thereof.

In some aspects, the present disclosure provides a compound of the present disclosure or a pharmaceutically acceptable salt thereof for use in treating or preventing BRCA1- or BRCA2-mutated cancer in a subject in need thereof.

In some aspects, the present disclosure provides a compound of the present disclosure or a pharmaceutically acceptable salt thereof for use in treating cancer in a subject in need thereof.

In some aspects, the present disclosure provides a compound of the present disclosure or a pharmaceutically acceptable salt thereof for use in treating HR-deficient cancer in a subject in need thereof.

In some aspects, the present disclosure provides a compound of the present disclosure or a pharmaceutically acceptable salt thereof for use in treating BRCA1- or BRCA2-mutated cancer in a subject in need thereof.

In some aspects, the present disclosure provides use of a compound of the present disclosure or a pharmaceutically acceptable salt thereof in the manufacture of a medicament for modulating PARP1 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing cancer in a subject in need thereof.

In some aspects, the present disclosure provides use of a compound of the present disclosure or a pharmaceutically acceptable salt thereof in the manufacture of a medicament for treating or preventing HR-deficient cancer in a subject in need thereof.

In some aspects, the present disclosure provides use of a compound of the present disclosure or a pharmaceutically acceptable salt thereof in the manufacture of a medicament for treating or preventing BRCA1- or BRCA2-mutated cancer in a subject in need thereof.

In some aspects, the present disclosure provides use of a compound of the present disclosure or a pharmaceutically acceptable salt thereof in the manufacture of a medicament for treating cancer in a subject in need thereof.

In some aspects, the present disclosure provides use of a compound of the present disclosure or a pharmaceutically acceptable salt thereof in the manufacture of a medicament for treating HR-deficient cancer in a subject in need thereof.

In some aspects, the present disclosure provides use of a compound of the present disclosure or a pharmaceutically acceptable salt thereof in the manufacture of a medicament for treating BRCA1- or BRCA2-mutated cancer in a subject in need thereof.

The present disclosure provides compounds that function as modulators of PARP1 activity.

In some embodiments, the compounds of the present disclosure are inhibitors of PARP1.

In some embodiments, the modulation of PARP1 is inhibition of PARP1.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PARP1 activity is implicated in a patient in need of such treatment, said method comprising administering to said patient a therapeutically effective amount of a compound, or a pharmaceutically acceptable salt thereof, or a pharmaceutical composition as defined herein.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced).

Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which PARP1 activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardization of in vitro and in vivo test systems for the evaluation of the effects of modulators of PARP1 activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

It is understood that the neutral compounds of Formula (I) may be converted to the corresponding pharmaceutically acceptable salts of the compounds using techniques in the art (e.g., by saponification of an ester to the carboxylic acid salt, or by hydrolyzing an amide to form a corresponding carboxylic acid and then converting the carboxylic acid to a carboxylic acid salt).

### Abbreviations:

¹H Proton
ACN Acetonitrile
Aq. Aqueous
Boc tert-Butyl carbamate
Brine Saturated sodium chloride solution
DCM Dichloromethane
DIPEA N,N-diisopropylethylamine
DMA N,N-dimethylacetamide
DMF N,N-dimethylformamide
DMSO Dimethylsulfoxide
d Doublet
dd Doublet of doublets
EA Ethyl acetate
FA Formic acid
HPLC High-performance liquid chromatography
J J value
LC/MS Liquid chromatography/mass spectrometry
MeOH Methanol
m Multiplet
m/z Mass/charge ratio
NMR Nuclear magnetic resonance
Prep Preparative
RT Room temperature
s Singlet
TEA Triethylamine
TFA Trifluoroacetic acid
t Triplet
   XantPhos 4,5-Bis(diphenylphosphino)-9,9-dimethylxanthene

3-Ethyl-7-(hydroxymethyl)-1,5-naphthyridin-2(1H)-one was either purchased or prepared following Johannes et al., US2021/0040084 A1.

### Example S—1: 5-(6-((7-ethyl-6-oxo-5,6-dihydro-1,5-naphthyridin-3-yl)methyl)-2,6-diazaspiro[3.3]heptan-2-yl)-N-methylpicolinamide formate, (Compound No. 9

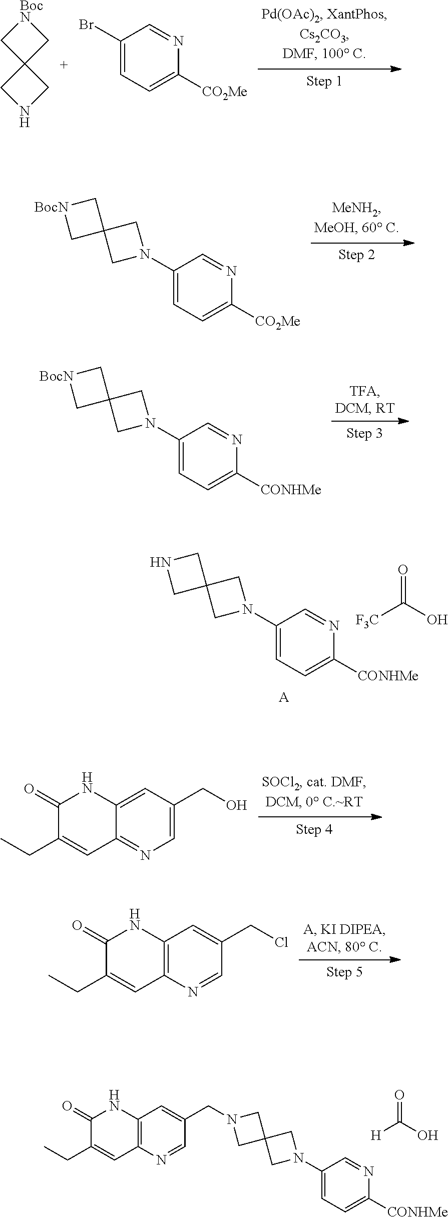

Step 1: Preparation of tert-butyl 6-(6-(methoxycarbonyl)pyridin-3-yl)-2,6-diazaspiro[3.3]heptane-2-carboxylate: A mixture of methyl 5-bromopicolinate (1.00 g, 4.6 mmol), tert-butyl 2,6-diazaspiro[3.3]heptane-2-carboxylate (1.13 g, 5.7 mmol), XantPhos (0.54 g, 0.93 mmol), Cs₂CO₃ (3.02 g, 9.3 mmol) and Pd(OAc)₂ (0.1 g, 0.45 mmol) in DMF (7 mL) was stirred at 100° C. for 16 h under a nitrogen atmosphere. The mixture was diluted with ethyl acetate, washed with brine and dried over anhydrous Na₂SO₄. The organic layer was filtered, and the filtrate was concentrated in vacuo. Purification by Combi-Flash chromatography afforded tert-butyl 6-(6-(methoxycarbonyl)pyridin-3-yl)-2,6-diazaspiro[3.3]heptane-2-carboxylate (380 mg, 24%). LC/MS m/z: 334.2 [M+H]⁺.

Step 2: Preparation of tert-butyl 6-(6-(methylcarbamoyl)pyridin-3-yl)-2,6-diazaspiro[3.3]heptane-2-carboxylate: A solution of tert-butyl 6-(6-(methoxycarbonyl)pyridin-3-yl)-2,6-diazaspiro[3.3]heptane-2-carboxylate (380 mg, 1.14 mmol) in MeNH₂ (10 mL, 33% in MeOH) was stirred at 60° C. for 8 h in a sealed tube. The mixture was concentrated and purified by Combi-Flash chromatography to afford tert-butyl 6-(6-(methylcarbamoyl)pyridin-3-yl)-2,6-diazaspiro[3.3]heptane-2-carboxylate (300 mg, 79%). LC/MS m/z: 333.2 [M+H]⁺.

Step 3: Preparation of N-methyl-5-(2,6-diazaspiro[3.3]heptan-2-yl)picolinamide trifluoroacetate: To a solution of tert-butyl 6-(6-(methylcarbamoyl)pyridin-3-yl)-2,6-diazaspiro[3.3]heptane-2-carboxylate (70 mg, 0.21 mmol) in DCM (2 mL) was added TFA (2 mL) and the reaction mixture was stirred at RT for 1 h. The reaction was concentrated to afford N-methyl-5-(2,6-diazaspiro[3.3]heptan-2-yl)picolinamide trifluoroacetate (40 mg) which was used without further purification. LC/MS m/z: 233.2 [M+H]⁺.

Step 4: Preparation of 7-(chloromethyl)-3-ethyl-1,5-naphthyridin-2(1H)-one: To a solution of 3-ethyl-7-(hydroxymethyl)-1H-1,5-naphthyridin-2-one (50 mg, 0.24 mmol) in DCM (5 mL) was added SOCl₂ (44 mg, 0.37 mmol) and a drop of DMF at 0° C. The reaction was stirred at RT for 3 h. The resulting mixture was poured into ice water and extracted with ethyl acetate. The combined organic layers were washed with brine, dried over anhydrous Na₂SO₄, filtered and the filtrate was concentrated in vacuo. Purification by Combi-Flash chromatography afforded 7-(chloromethyl)-3-ethyl-1,5-naphthyridin-2(1H)-one (43 mg, 79%). LC/MS m/z: 223.1 [M+H]⁺.

Step 5: Preparation of 5-(6-((7-ethyl-6-oxo-5,6-dihydro-1,5-naphthyridin-3-yl)methyl)-2,6-diazaspiro[3.3]heptan-2-yl)-N-methylpicolinamide formate: To a solution of N-methyl-5-(2,6-diazaspiro[3.3]heptan-2-yl)picolinamide trifluoroacetate (40 mg, 0.12 mmol), 7-(chloromethyl)-3-ethyl-1H-1,5-naphthyridin-2-one (38 mg, 0.17 mmol) in ACN (2 mL) was added KI (43 mg, 0.26 mmol) and DIPEA (223 mg, 1.7 mmol) and the resultant mixture was stirred at 80° C. for 2 h. The mixture was concentrated in vacuo and purification by prep-HPLC afforded 5-(6-((7-ethyl-6-oxo-5,6-dihydro-1,5-naphthyridin-3-yl)methyl)-2,6-diazaspiro[3.3]heptan-2-yl)-N-methylpicolinamide formate (20 mg, 25%). LC/MS m/z: 419.2 [M+H]⁺; ¹H NMR (400 MHz, DMSO-d₆) δ: 11.88 (s, 1H), 8.41-8.29 (m, 2H), 8.14 (s, 1H, FA), 7.80 (d, J=8.5 Hz, 1H), 7.77-7.69 (m, 2H), 7.56 (s, 1H), 6.88 (dd, J=8.6, 2.8 Hz, 1H), 4.06 (s, 4H), 3.73 (s, 2H), 3.45 (s, 4H), 2.77 (d, J=4.8 Hz, 3H), 2.58-2.52 (m, 2H), 1.18 (t, J=7.4 Hz, 3H).

### Example S—2: 5-(4-((7-ethyl-6-oxo-5H-1,5-naphthyridin-3-yl)methyl)-1,4-diazepan-1-yl)-N-methylpyridine-2-carboxamide, (Compound No. 6

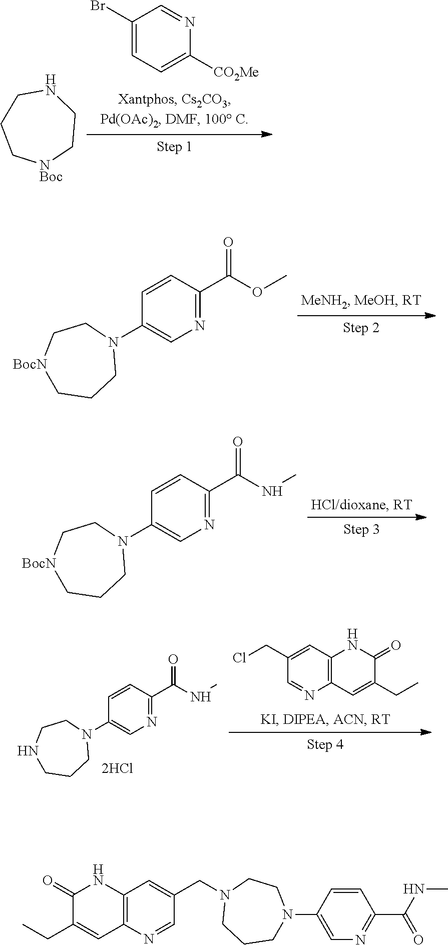

Step 1: Preparation of tert-butyl 4-(6-(methoxycarbonyl)pyridin-3-yl)-1,4-diazepane-1-carboxylate: A mixture of tert-butyl 1,4-diazepane-1-carboxylate (200 mg, 1.0 mmol), methyl 5-bromopyridine-2-carboxylate (216 mg, 1.0 mmol), XantPhos (58 mg, 0.10 mmol), Pd(OAc)₂ (22 mg, 0.10 mmol) and Cs₂CO₃ (652 mg, 2.00 mmol) in DMF (4 mL) was stirred at 100° C. for 16 h under a nitrogen atmosphere. The reaction was quenched with water (50 mL) and extracted with ethyl acetate. The combined organic layers were washed with brine and concentrated. The residue was purified by silica gel column chromatography to afford tert-butyl 4-(6-(methoxycarbonyl)pyridin-3-yl)-1,4-diazepane-1-carboxylate (170 mg, 51%). LC/MS m/z: 336.2 [M+H]⁺.

Step 2: Preparation of tert-butyl 4-(6-(methylcarbamoyl)pyridin-3-yl)-1,4-diazepane-1-carboxylate: A solution of tert-butyl 4-(6-(methoxycarbonyl)pyridin-3-yl)-1,4-diazepane-1-carboxylate (170 mg, 0.51 mmol) and methylamine in MeOH (33%, 3 mL) was stirred at RT for 1 h before it was concentrated to afford tert-butyl 4-(6-(methylcarbamoyl)pyridin-3-yl)-1,4-diazepane-1-carboxylate (130 mg) which was used without further purification. LC/MS m/z: 335.2 [M+H]⁺.

Step 3: Preparation of 5-(1,4-diazepan-1-yl)-N-methylpicolinamide dihydrochloride: A solution of tert-butyl 4-(6-(methylcarbamoyl)pyridin-3-yl)-1,4-diazepane-1-carboxylate (130 mg, 0.39 mmol) in 1,4-dioxane/HCl (4M, 4 mL) was stirred at RT for 5 h before it was concentrated to afford 5-(1,4-diazepan-1-yl)-N-methylpicolinamide dihydrochloride (100 mg) which was used without further purification. LC/MS m/z: 235.1 [M+H]⁺.

Step 4: Preparation of 5-(4-((7-ethyl-6-oxo-5H-1,5-naphthyridin-3-yl)methyl)-1,4-diazepan-1-yl)-N-methylpyridine-2-carboxamide: To a solution of 7-(chloromethyl)-3-ethyl-1H-1,5-naphthyridin-2-one (60 mg, 0.26 mmol) in ACN (5 mL) were added 5-(1,4-diazepan-1-yl)-N-methylpicolinamide dihydrochloride (100 mg, 0.33 mmol), KI (67 mg, 0.40 mmol) and DIPEA (348 mg, 2.69 mmol). The resulting solution was stirred at RT for 5 h before it was concentrated and purified by Prep-HPLC to afford impure material. The impure material was dissolved in water and adjusted to pH=7 with NaHCO₃ solution. The solid was collected by filtration to afford 5-(4-((7-ethyl-6-oxo-5H-1,5-naphthyridin-3-yl)methyl)-1,4-diazepan-1-yl)-N-methylpyridine-2-carboxamide (34 mg, 30%). LC/MS m/z: 421.1 [M+H]⁺; ¹H NMR (400 MHz, DMSO-d₆) δ 11.83 (s, 1H), 8.36-8.33 (m, 1H), 8.31-8.26 (m, 1H), 8.09-8.04 (m, 1H), 7.81-7.76 (m, 1H), 7.74 (s, 1H), 7.60 (s, 1H), 7.20-7.15 (m, 1H), 3.73 (s, 2H), 3.65-3.56 (m, 4H), 2.81-2.72 (m, 5H), 2.59-2.52 (m, 4H), 1.92-1.85 (m, 2H), 1.18 (t, J=7.4 Hz, 3H).

### Example S—3: 5-(5-((7-ethyl-6-oxo-5,6-dihydro-1,5-naphthyridin-3-yl)methyl)hexahydropyrrolo[3,4-c]pyrrol-2(1H)-yl)-N-methylpicolinamide, (Compound No. 7

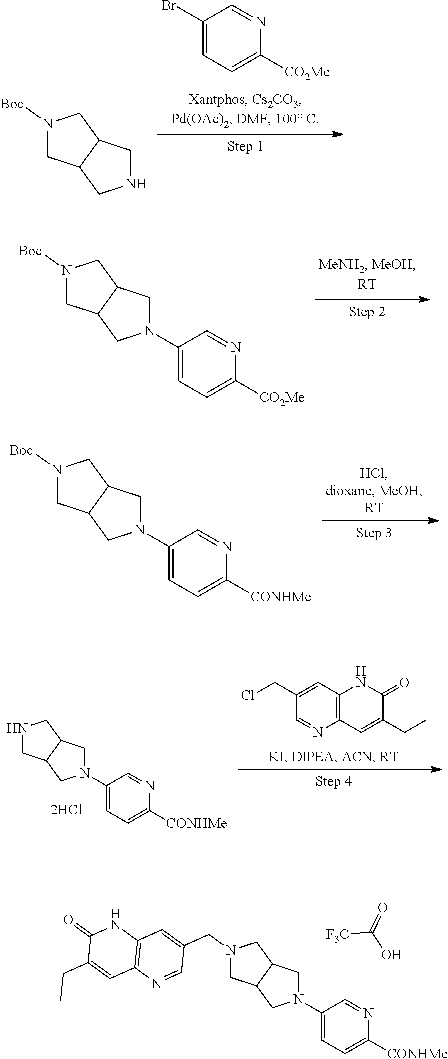

Step 1: Preparation of tert-butyl 5-(6-(methoxycarbonyl)pyridin-3-yl)hexahydropyrrolo[3,4-c]pyrrole-2(1H)-carboxylate: To a stirred solution of tert-butyl hexahydropyrrolo[3,4-c]pyrrole-2(1H)-carboxylate (2 g, 9.4 mmol) in DMF (20 mL) was added methyl 5-bromopyridine-2-carboxylate (2.03 g, 9.4 mmol), XantPhos (2.18 g, 3.76 mmol), Pd(OAc)₂ (0.42 g, 1.88 mmol) and Cs₂CO₃ (6.13 g, 18.8 mmol) at 25° C. The reaction was warmed to 100° C. and stirred at that temperature for 3 h. The reaction mixture was concentrated under reduced pressure and purified by silica gel column chromatography to afford tert-butyl 5-(6-(methoxycarbonyl)pyridin-3-yl) hexahydropyrrolo[3,4-c] pyrrole-2(1H)-carboxylate (1.4 g, 43%). LC/MS m/z: 348.2 [M+H]⁺.

Step 2: Preparation of tert-butyl 5-(6-(methylcarbamoyl)pyridin-3-yl)hexahydropyrrolo[3,4-c]pyrrole-2(1H)-carboxylate: A solution of tert-butyl 5-(6-(methoxycarbonyl) pyridin-3-yl)hexahydropyrrolo[3,4-c]pyrrole-2(1H)-carboxylate (1.4 g, 4.03 mmol) in MeNH₂ (20 mL, 4M in MeOH) was stirred at RT for 18 h. The reaction mixture was concentrated under reduced pressure to afford tert-butyl 5-(6-(methylcarbamoyl)pyridin-3-yl)hexahydropyrrolo[3,4-c]pyrrole-2(1H)-carboxylate (1.3 g) which was used without further purification. LC/MS m/z: 347.1 [M+H]⁺.

Step 3: Preparation of 5-(hexahydro-1H-pyrrolo[3,4-c]pyrrol-2-yl)-N-methylpyridine-2-carboxamide dihydrochloride: To a stirred solution of tert-butyl 5-(6-(methylcarbamoyl)pyridin-3-yl)hexahydropyrrolo[3,4-c]pyrrole-2(1H)-carboxylate (1.3 g, 3.75 mmol) in MeOH (8 mL) was added HCl in dioxane (4 mL, 4M). The reaction was stirred at RT for 18 h. The reaction mixture was concentrated under reduced pressure to afford 5-(hexahydro-1H-pyrrolo[3,4-c]pyrrol-2-yl)-N-methylpyridine-2-carboxamide dihydrochloride (1.1 g) which was used without further purification. LC/MS m/z: 247.1 [M+H]⁺.

Step 4: Preparation of 5-(5-((7-ethyl-6-oxo-5,6-dihydro-1,5-naphthyridin-3-yl) methyl) hexahydropyrrolo[3,4-c] pyrrol-2(1H)-yl)-N-methylpicolinamide trifluoroacetate: To a stirred solution of 7-(chloromethyl)-3-ethyl-1H-1,5-naphthyridin-2-one (110 mg, 0.45 mmol) in ACN (3 mL) was added 5-(hexahydro-1H-pyrrolo[3,4-c]pyrrol-2-yl)-N-methylpyridine-2-carboxamide dihydrochloride (110 mg, 0.34 mmol), KI (111 mg, 0.67 mmol) and DIPEA (580 mg, 4.49 mmol). The reaction was stirred at RT for 18 h. The reaction mixture was quenched with aq. NH₄Cl, extracted with DCM (20 mL×2). The combined organic layers were washed with brine, dried over anhydrous Na₂SO₄, filtered and the filtrate was concentrated under reduced pressure. Purification by Prep-HPLC afforded 5-(5-[(7-ethyl-6-oxo-5H-1,5-naphthyridin-3-yl) methyl]-hexahydropyrrolo[3,4-c] pyrrol-2-yl)-N-methylpyridine-2-carboxamide trifluoroacetate (15 mg, 8%). LC/MS m/z: 433.3 [M+H]⁺; ¹H NMR (400 MHz, DMSO-d₆) δ 11.74 (s, 1H), 8.44-8.27 (m, 2H), 7.96 (d, J=2.7 Hz, 1H), 7.82 (d, J=8.7 Hz, 1H), 7.72 (s, 1H), 7.58 (s, 1H), 7.06 (dd, J=8.7, 2.8 Hz, 1H), 3.68 (s, 2H), 3.62-3.49 (m, 2H), 3.21 (dd, J=9.9, 3.0 Hz, 2H), 2.95 (s, 2H), 2.78 (d, J=4.8 Hz, 3H), 2.67-2.57 (m, 2H), 2.55-2.53 (m, 2H), 2.51-2.53 (m, 2H), 1.17 (t, J=7.4 Hz, 3H).

### Example S—4: 5-((3-(((7-ethyl-6-oxo-5,6-dihydro-1,5-naphthyridin-3-yl)methyl)amino)cyclobutyl)amino)-N-methylpicolinamide, (Compound No. 29

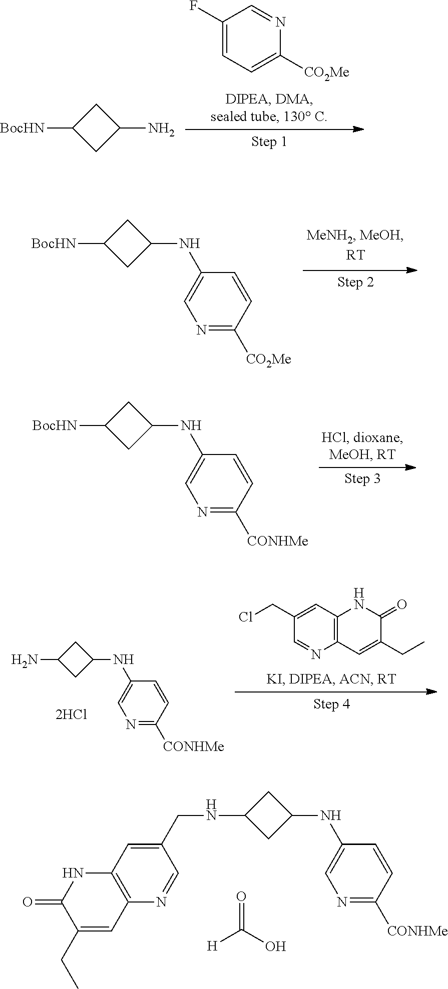

Step 1: Preparation of methyl 5-((3-((tert-butoxycarbonyl)amino)cyclobutyl)amino)picolinate: To a stirred solution of tert-butyl (3-aminocyclobutyl)carbamate (300 mg, 1.6 mmol) in DMA (3 mL) was added methyl 5-fluoropyridine-2-carboxylate (248 mg, 1.6 mmol) and DIPEA (1 g, 8.0 mmol) at 25° C. The reaction was warmed to 130° C. in a sealed tube and stirred at that temperature for 18 h. The reaction mixture was quenched with water, extracted with ethyl acetate. The combined organic layers were washed with brine, dried over Na₂SO₄, filtered and the filtrate was concentrated under reduced pressure. Purification by silica gel column chromatography afforded methyl 5-((3-((tert-butoxycarbonyl)amino)cyclobutyl)amino)picolinate (350 mg, 70%). LC/MS m/z: 322.1 [M+H]⁺.

Step 2: Preparation of tert-butyl (3-((6-(methylcarbamoyl)pyridin-3-yl)amino)cyclobutyl)carbamate: A solution of methyl 5-((3-((tert-butoxycarbonyl)amino)cyclobutyl)amino)picolinate (350 mg, 1.09 mmol) in MeNH₂ (4 mL, 33% in MeOH) was stirred at RT for 18 h. The reaction mixture was concentrated under reduced pressure to afford tert-butyl (3-((6-(methylcarbamoyl)pyridin-3-yl)amino)cyclobutyl)carbamate (320 mg) which was used without further purification. LC/MS m/z: 321.1 [M+H]⁺.

Step 3: Preparation of 5-((3-aminocyclobutyl)amino)-N-methylpicolinamide dihydrochloride: To a stirred solution of tert-butyl (3-((6-(methylcarbamoyl)pyridin-3-yl)amino)cyclobutyl)carbamate (320 mg, 1 mmol) in MeOH (2 mL) was added HCl in dioxane (1 mL, 4M). The reaction was stirred at RT for 18 h. The reaction mixture was concentrated under reduced pressure to afford 5-((3-aminocyclobutyl)amino)-N-methylpicolinamide dihydrochloride (260 mg) which was used without further purification. LC/MS m/z: 221.2 [M+H]⁺.

Step 4: Preparation of 5-((3-(((7-ethyl-6-oxo-5,6-dihydro-1,5-naphthyridin-3-yl)methyl)amino)cyclobutyl)amino)-N-methylpicolinamide formate: To a stirred solution of 7-(chloromethyl)-3-ethyl-1H-1,5-naphthyridin-2-one (110 mg, 0.45 mmol) in ACN (1 mL) was added 5-((3-aminocyclobutyl)amino)-N-methylpicolinamide dihydrochloride (105 mg, 0.36 mmol), KI (201 mg, 1.21 mmol) and DIPEA (261 mg, 2.02 mmol). The reaction was stirred at RT for 18 h. The reaction mixture was quenched with water and extracted with DCM. The combined organic layers were washed with brine, dried over anhydrous Na₂SO₄, filtered and the filtrate was concentrated under reduced pressure. Purification by Prep-HPLC afforded 5-((3-(((7-ethyl-6-oxo-5,6-dihydro-1,5-naphthyridin-3-yl)methyl)amino)cyclobutyl)amino)-N-methylpicolinamide formate (11 mg, 7%). LC/MS m/z: 407.1 [M+H]⁺; ¹H NMR (400 MHz, MeOH-d₄) δ 8.53 (d, J=23.7 Hz, 1H), 8.38 (s, 1H, FA), 7.93 (d, J=2.4 Hz, 1H), 7.82 (dd, J=19.8, 11.0 Hz, 3H), 6.89 (dd, J=8.6, 2.5 Hz, 1H), 4.13 (s, 3H), 3.89-3.72 (m, 1H), 2.94 (s, 3H), 2.69 (q, J=7.4 Hz, 2H), 2.60-2.49 (m, 2H), 2.33-2.37 (m, 2H), 1.37-1.13 (m, 3H).

### Example S—5: 7-({6-[(2,4-difluorophenyl)carbonyl]-2,6-diazaspiro[3.3]heptan-2-yl}methyl)-3-ethyl-1H-1,5-naphthyridin-2-one formate, (Compound No. 181

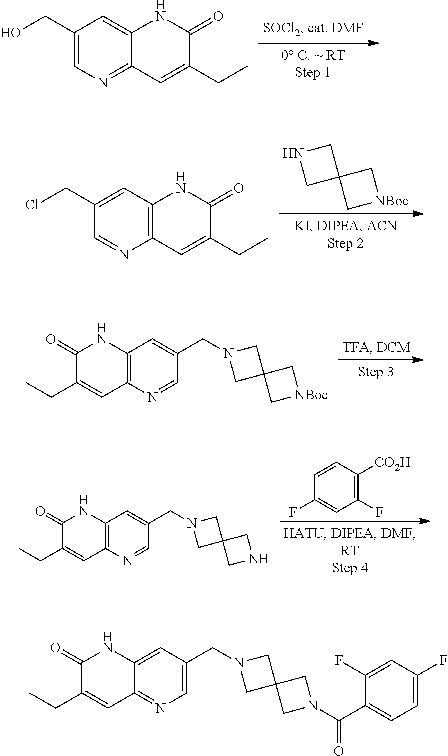

Step 1: Preparation of 7-(chloromethyl)-3-ethyl-1H-1,5-naphthyridin-2-one: To a solution of 3-ethyl-7-(hydroxymethyl)-1H-1,5-naphthyridin-2-one (500 mg, 2.45 mmol) in DCM/DMF (5 ml/0.5 ml) was added SOCl₂ (583 mg, 4.90 mmol) at 0° C. The resulting solution was stirred at RT for 1 h. The solvent was removed in vacuo to afford 7-(chloromethyl)-3-ethyl-1H-1,5-naphthyridin-2-one (500 mg, 92%). LC/MS m/z: 223.1 [M+H]⁺.

Step 2: Preparation of tert-butyl 6-[(7-ethyl-6-oxo-5H-1,5-naphthyridin-3-yl)methyl]-2,6-diazaspiro[3.3]heptane-2-carboxylate: A mixture of 7-(chloromethyl)-3-ethyl-1H-1,5-naphthyridin-2-one (110 mg, 0.49 mmol), tert-butyl 2,6-diazaspiro[3.3]heptane-2-carboxylate (97 mg, 0.49 mmol), KI (123 mg, 0.74 mmol) and DIPEA (320 mg, 2.48 mmol) in ACN (8 mL) was stirred at RT for 20 h. Then the solvent was removed in vacuo. The crude product was purified by silica gel column chromatography to afford tert-butyl 6-[(7-ethyl-6-oxo-5H-1,5-naphthyridin-3-yl)methyl]-2,6-diazaspiro[3.3]heptane-2-carboxylate (130 mg, 69%). LC/MS m/z: 385.2 [M+H]⁺.

Step 3: Preparation of 7-{2,6-diazaspiro[3.3]heptan-2-ylmethyl}-3-ethyl-1H-1,5-naphthyridin-2-one: To a solution of tert-butyl 6-[(7-ethyl-6-oxo-5H-1,5-naphthyridin-3-yl)methyl]-2,6-diazaspiro[3.3]heptane-2-carboxylate (50 mg, 0.13 mmol) in DCM (2 mL) was added TFA (0.8 mL). The resulting solution was stirred at RT for 1 h. After completion, the reaction mixture was concentrated and purified by Prep-HPLC to give impure product (30 mg, impure). The impure product was dissolved in water and adjusted to pH 7 with NaHCO₃ solution, and the solid was collected by filtration to afford 7-{2,6-diazaspiro[3.3]heptan-2-ylmethyl}-3-ethyl-1H-1,5-naphthyridin-2-one (7.2 mg, 19%). LC/MS m/z: 285.2 [M+H]⁺; ¹H NMR (400 MHz, DMSO-d₆) δ 8.26 (s, 1H), 7.68 (s, 1H), 7.49 (s, 1H), 3.73 (s, 1H), 3.55 (s, 2H), 3.51-3.40 (m, 4H), 3.22-3.17 (m, 4H), 2.54-2.47 (m, 2H), 1.13 (t, J=7.4 Hz, 3H).

Step 4: Preparation of 7-({6-[(2,4-difluorophenyl)carbonyl]-2,6-diazaspiro[3.3]heptan-2-yl}methyl)-3-ethyl-1H-1,5-naphthyridin-2-one formate: A mixture of 7-{2,6-diazaspiro[3.3]heptan-2-ylmethyl}-3-ethyl-1H-1,5-naphthyridin-2-one (30 mg, 0.11 mmol), 2,4-difluorobenzoic acid (17 mg, 0.11 mmol), HATU (60 mg, 0.16 mmol) and DIPEA (28 mg, 0.21 mmol) in DCM (2 mL) was stirred at RT for 3 h. The reaction was concentrated and the residue was purified by Prep-HPLC to afford 7-({6-[(2,4-difluorophenyl)carbonyl]-2,6-diazaspiro[3.3]heptan-2-yl}methyl)-3-ethyl-1H-1,5-naphthyridin-2-one formate (6.8 mg, 13%). LC/MS m/z: 425.1 [M+H]⁺; ¹H NMR (400 MHz, CDCl₃) δ 8.44 (s, 1H), 7.83 (s, 1H), 7.67 (s, 1H), 7.60-7.51 (m, 1H), 6.98-6.89 (m, 1H), 6.88-6.79 (m, 1H), 4.27 (d, J=13.3 Hz, 4H), 3.86 (s, 2H), 3.74-3.52 (m, 4H), 2.77-2.61 (m, 2H), 1.31-1.24 (m, 3H).

### Example S—6: 5-(6-((7-ethyl-6-oxo-5,6-dihydro-1,5-naphthyridin-3-yl)methyl)-2,6-diazaspiro[3.3]heptan-2-yl)-N,6-dimethylpicolinamide formate, (Compound No. 139

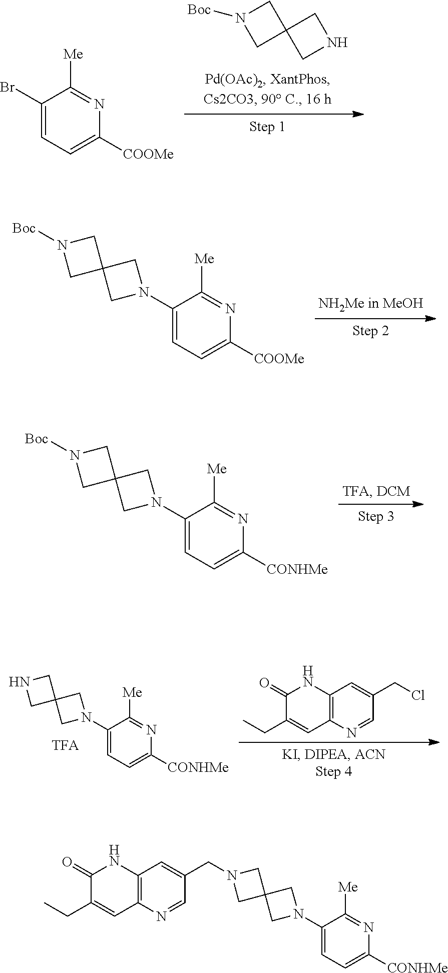

Step 1: Preparation of tert-butyl 6-(6-(methoxycarbonyl)-2-methylpyridin-3-yl)-2,6-diazaspiro[3.3]heptane-2-carboxylate: To a solution of methyl 5-bromo-6-methylpicolinate (110 mg, 0.48 mmol) and tert-butyl 2,6-diazaspiro[3.3]heptane-2-carboxylate (142 mg, 0.72 mmol) in dioxane (5 mL) was added Pd(OAc)₂ (10.9 mg, 0.048 mmol), XantPhos (55.5 mg, 0.096 mmol) and Cs₂CO₃ (469 mg, 1.44 mmol). The reaction solution was stirred at 90° C. for 16 h under nitrogen before it was concentrated under reduced pressure. The residue was purified by silica gel chromatography to afford tert-butyl 6-(6-(methoxycarbonyl)-2-methylpyridin-3-yl)-2,6-diazaspiro[3.3]heptane-2-carboxylate (150 mg, 90%). LC/MS m/z: 348.1 [M+H]⁺.

Step 2: Preparation of tert-butyl 6-(2-methyl-6-(methylcarbamoyl)pyridin-3-yl)-2,6-diazaspiro[3.3]heptane-2-carboxylate: To a stirred solution of tert-butyl 6-(6-(methoxycarbonyl)-2-methylpyridin-3-yl)-2,6-diazaspiro[3.3]heptane-2-carboxylate (150 mg, 0.43 mmol) in MeOH (2 mL) was added MeNH₂ (33% in MeOH, 2 mL). The reaction was stirred at 25° C. for 18 h before it was concentrated under reduced pressure and purified by silica gel chromatography to afford tert-butyl 6-(2-methyl-6-(methylcarbamoyl)pyridin-3-yl)-2,6-diazaspiro[3.3]heptane-2-carboxylate (149 mg, 100%). LC/MS m/z: 347.2 [M+H]⁺.

Step 3: Preparation of N,6-dimethyl-5-(2,6-diazaspiro[3.3]heptan-2-yl)picolinamide trifluoroacetate: To a stirred solution of tert-butyl 6-(2-methyl-6-(methylcarbamoyl)pyridin-3-yl)-2,6-diazaspiro[3.3]heptane-2-carboxylate (149 mg, 0.43 mmol) in DCM (6 mL) was added TFA (2 mL). The reaction was stirred at 25° C. for 4 h. The reaction mixture was concentrated under reduced to afford N,6-dimethyl-5-(2,6-diazaspiro[3.3]heptan-2-yl)picolinamide trifluoroacetate (88 mg, crude). LC/MS m/z: 247.2 [M+H]⁺.

Step 4: Preparation of 5-(6-((7-ethyl-6-oxo-5,6-dihydro-1,5-naphthyridin-3-yl)methyl)-2,6-diazaspiro[3.3]heptan-2-yl)-N,6-dimethylpicolinamide formate: To a solution of 7-(chloromethyl)-3-ethyl-1,5-naphthyridin-2(1H)-one (54 mg, 0.24 mmol) in ACN (10 mL) were added N,6-dimethyl-5-(2,6-diazaspiro[3.3]heptan-2-yl)picolinamide trifluoroacetic acid (88 mg, crude), KI (60.3 mg, 0.36 mmol) and DIPEA (309.6 mg, 2.4 mmol). The resulting solution was stirred at RT for 5 h before it was concentrated and purified by Prep-HPLC to afford 5-(6-((7-ethyl-6-oxo-5,6-dihydro-1,5-naphthyridin-3-yl)methyl)-2,6-diazaspiro[3.3]heptan-2-yl)-N,6-dimethylpicolinamide formate (18.7 mg, 16%). LC/MS m/z: 433.1 [M+H]⁺; ¹H NMR (400 MHz, MeOH-d₄) δ 8.52 (s, 1H), 7.86 (s, 1H), 7.83-7.73 (m, 2H), 6.87 (d, J=8.4 Hz, 1H), 4.28-4.12 (m, 6H), 4.04-3.89 (m, 4H), 2.94 (s, 3H), 2.66 (q, J=7.4 Hz, 2H), 2.49 (s, 3H), 1.30 (t, J=7.4 Hz, 3H).

### Example S—7: 5-(8-((7-ethyl-6-oxo-5,6-dihydro-1,5-naphthyridin-3-yl)methyl)-3,8-diazabicyclo[3.2.1]octan-3-yl)-N-methylpicolinamide, (Compound No. 3

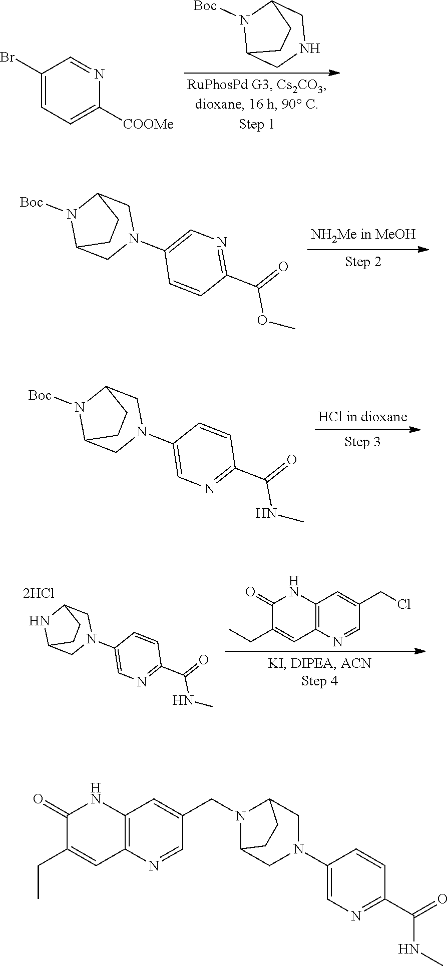

Step 1: Preparation of tert-butyl 3-(6-(methoxycarbonyl)pyridin-3-yl)-3,8-diazabicyclo[3.2.1]octane-8-carboxylate: To a solution of methyl 5-bromopicolinate (200 mg, 0.93 mmol) and tert-butyl 3,8-diazabicyclo[3.2.1]octane-8-carboxylate (238 mg, 1.12 mmol) in dioxane (10 mL) were added RuphosPdG3 (78 mg, 0.093 mmol) and Cs₂CO₃ (606 mg, 1.86 mmol). The reaction solution was stirred at 90° C. for 16 h under nitrogen before it was concentrated under reduced pressure. The residue was purified by silica gel chromatography to afford tert-butyl 3-(6-(methoxycarbonyl)pyridin-3-yl)-3,8-diazabicyclo[3.2.1]octane-8-carboxylate (185 mg, 57%) as a white solid. LC/MS m/z: 348.1 [M+H]⁺.

Step 2: Preparation of tert-butyl 3-(6-(methylcarbamoyl)pyridin-3-yl)-3,8-diazabicyclo[3.2.1]octane-8-carboxylate: To a stirred solution of tert-butyl 3-(6-(methoxycarbonyl)pyridin-3-yl)-3,8-diazabicyclo[3.2.1]octane-8-carboxylate (185 mg, 0.53 mmol) in MeOH (2 mL) was added MeNH₂ (33% in MeOH, 2 mL). The reaction was stirred at 25° C. for 18 h before it was concentrated under reduced pressure and purified by silica gel chromatography to afford tert-butyl 3-(6-(methylcarbamoyl)pyridin-3-yl)-3,8-diazabicyclo[3.2.1]octane-8-carboxylate (170 mg, 93%). LC/MS m/z: 347.2 [M+H]⁺.

Step 3: Preparation of 5-(3,8-diazabicyclo[3.2.1]octan-3-yl)-N-methylpicolinamide dihydrochloride: To a stirred solution of tert-butyl 3-(6-(methylcarbamoyl)pyridin-3-yl)-3,8-diazabicyclo[3.2.1]octane-8-carboxylate (170 mg, 0.49 mmol) in DCM (6 mL) was added HCl (2 mL, 4M in dioxane). The reaction was stirred at 25° C. for 4 h. The reaction mixture was concentrated under reduced pressure to afford 5-(3,8-diazabicyclo[3.2.1]octan-3-yl)-N-methylpicolinamide dihydrochloride (130 mg, crude). LC/MS m/z: 247.1 [M+H]⁺.

Step 4: Preparation of 5-(8-((7-ethyl-6-oxo-5,6-dihydro-1,5-naphthyridin-3-yl)methyl)-3,8-diazabicyclo[3.2.1]octan-3-yl)-N-methylpicolinamide: To a solution of 7-(chloromethyl)-3-ethyl-1,5-naphthyridin-2(1H)-one (54 mg, 0.24 mmol) in ACN (10 mL) were added 5-(3,8-diazabicyclo[3.2.1]octan-3-yl)-N-methylpicolinamide dihydrochloride (100 mg, crude), KI (60 mg, 0.36 mmol) and DIPEA (309.6 mg, 2.4 mmol). The resulting solution was stirred at RT for 5 h before it was concentrated and purified by Prep-HPLC to afford 5-(8-((7-ethyl-6-oxo-5,6-dihydro-1,5-naphthyridin-3-yl)methyl)-3,8-diazabicyclo[3.2.1]octan-3-yl)-N-methylpicolinamide (35 mg, 34%). LCMS: 433.2 [M+H]⁺; ¹H NMR (400 MHz, DMSO-d₆) δ 11.82 (s, 1H), 8.45-8.42 (m, 1H), 8.41-8.36 (m, 1H), 8.15 (d, J=2.6 Hz, 1H), 7.82 (d, J=8.8 Hz, 1H), 7.75 (s, 2H), 7.27 (dd, J=8.9, 2.8 Hz, 1H), 3.71 (s, 2H), 3.57 (d, J=9.6 Hz, 2H), 3.01 (d, J=9.4 Hz, 2H), 2.77 (d, J=4.8 Hz, 3H), 2.57-2.51 (m, 4H), 2.09-1.98 (m, 2H), 1.73-1.68 (m, 2H), 1.18 (t, J=7.4 Hz, 3H).

### Example S—8: 5-(3-((7-ethyl-6-oxo-5,6-dihydro-1,5-naphthyridin-3-yl)methyl)-3,8-diazabicyclo[3.2.1]octan-8-yl)-N-methylpicolinamide, (Compound No. 8

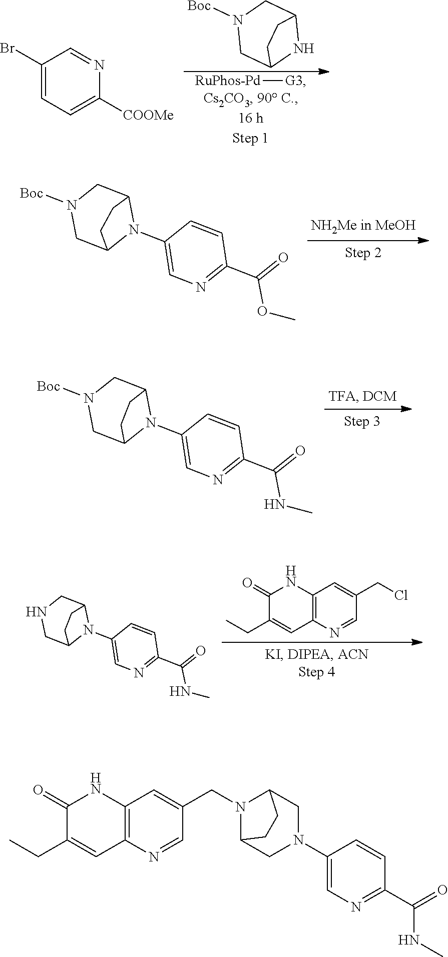

Step 1: Preparation of tert-butyl 8-(6-(methoxycarbonyl)pyridin-3-yl)-3,8-diazabicyclo[3.2.1]octane-3-carboxylate: To a solution of methyl 5-bromopicolinate (200 mg, 0.93 mmol), tert-butyl 3,8-diazabicyclo[3.2.1]octane-3-carboxylate (197 mg, 0.93 mmol) in dioxane (2 mL) were added Cs₂CO₃ (603 mg, 1.85 mmol) and RuPhos Pd G3 (78 mg, 0.093 mmol). The reaction was stirred at 90° C. for 16 h under nitrogen before it was concentrated and the residue was purified by Combi-Flash to afford tert-butyl 8-(6-(methoxycarbonyl)pyridin-3-yl)-3,8-diazabicyclo[3.2.1]octane-3-carboxylate (260 mg, 80%). LC/MS m/z: 348.1 [M+H]⁺.

Step 2: Preparation of tert-butyl 8-(6-(methylcarbamoyl)pyridin-3-yl)-3,8-diazabicyclo[3.2.1]octane-3-carboxylate: A solution of tert-butyl 8-(6-(methoxycarbonyl)pyridin-3-yl)-3,8-diazabicyclo[3.2.1]octane-3-carboxylate (260 mg, 0.75 mmol) in MeNH₂ (5 mL, 33% in MeOH) was stirred at 20° C. for 16 h in a sealed tube. The reaction was concentrated to afford tert-butyl 8-(6-(methylcarbamoyl)pyridin-3-yl)-3,8-diazabicyclo[3.2.1]octane-3-carboxylate (300 mg, crude). LC/MS m/z: 347.2 [M+H]⁺.

Step 3: Preparation of 5-(3,8-diazabicyclo[3.2.1]octan-8-yl)-N-methylpicolinamide trifluoroacetate: To a solution of tert-butyl 8-(6-(methylcarbamoyl)pyridin-3-yl)-3,8-diazabicyclo[3.2.1]octane-3-carboxylate (300 mg, crude) in DCM (3 mL) was added TFA (1 mL). The reaction mixture was stirred at 25° C. for 1 h before it was concentrated to afford 5-(3,8-diazabicyclo[3.2.1]octan-8-yl)-N-methylpicolinamide trifluoroacetate (580 mg, crude). LC/MS m/z: 247.1 [M+H]⁺.

Step 4: Preparation of 5-(3-((7-ethyl-6-oxo-5,6-dihydro-1,5-naphthyridin-3-yl)methyl)-3,8-diazabicyclo[3.2.1]octan-8-yl)-N-methylpicolinamide: To a solution of 5-(3,8-diazabicyclo[3.2.1]octan-8-yl)-N-methylpicolinamide trifluoroacetate (150 mg, crude) and 7-(chloromethyl)-3-ethyl-1,5-naphthyridin-2(1H)-one (33.4 mg, 0.15 mmol) in ACN (2 mL) were added KI (30 mg, 0.18 mmol) and DIPEA (97 mg, 0.75 mmol). The reaction was stirred at 20° C. for 16 h before it was concentrated and purified by prep-HPLC to afford 5-(3-((7-ethyl-6-oxo-5,6-dihydro-1,5-naphthyridin-3-yl)methyl)-3,8-diazabicyclo[3.2.1]octan-8-yl)-N-methylpicolinamide (37.1 mg, 57%). LC/MS m/z: 433.1 [M+H]⁺; ¹H NMR (400 MHz, MeOH-d₄) δ 8.40 (d, J=1.5 Hz, 1H), 8.13 (d, J=2.6 Hz, 1H), 7.84 (d, J=8.7 Hz, 1H), 7.79 (s, 1H), 7.71 (s, 1H), 7.21 (dd, J=8.8, 2.8 Hz, 1H), 4.39 (s, 2H), 3.55 (s, 2H), 2.91 (s, 3H), 2.67-2.57 (m, 4H), 2.52-2.47 (m, 2H), 2.17-2.10 (m, 2H), 2.03-1.95 (m, 2H), 1.25 (t, J=7.4 Hz, 3H).

### Example S—9: 5-(6-((7-ethyl-6-oxo-5,6-dihydro-1,5-naphthyridin-3-yl)methyl)-3,6-diazabicyclo[3.1.1]heptan-3-yl)-N-methylpicolinamide, (Compound No. 1

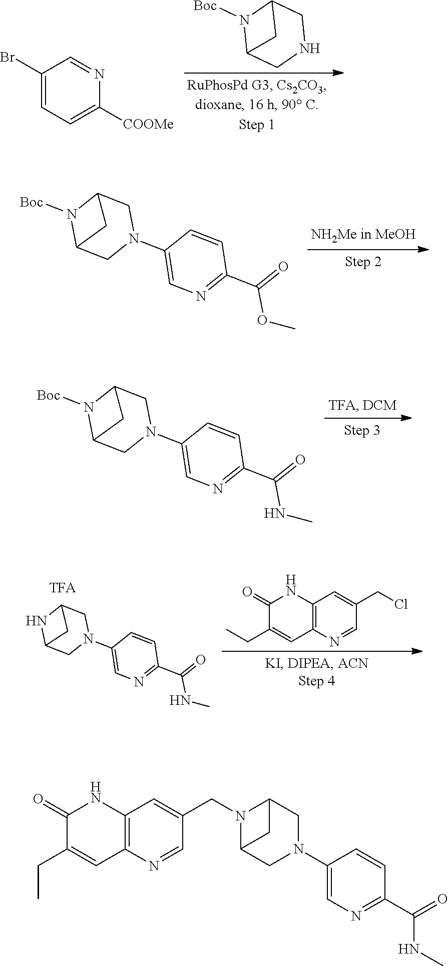

Step 1: Preparation of tert-butyl 3-(6-(methoxycarbonyl)pyridin-3-yl)-3,6-diazabicyclo[3.1.1]heptane-6-carboxylate: To a solution of methyl 5-bromopicolinate (250 mg, 1.16 mmol) and tert-butyl 3,6-diazabicyclo[3.1.1]heptane-6-carboxylate (230 mg, 1.16 mmol) in dioxane (10 mL) were added RuphosPdG3 (97 mg, 0.116 mmol) and Cs₂CO₃ (752 mg, 2.31 mmol). The reaction solution was stirred at 90° C. for 16 h under nitrogen before it was concentrated under reduced pressure. The residue was purified by silica gel chromatography to afford tert-butyl 3-(6-(methoxycarbonyl)pyridin-3-yl)-3,6-diazabicyclo[3.1.1]heptane-6-carboxylate (300 mg, 78%). LC/MS m/z: 334.1 [M+H]⁺.

Step 2: Preparation of tert-butyl 3-(6-(methylcarbamoyl)pyridin-3-yl)-3,6-diazabicyclo[3.1.1]heptane-6-carboxylate: To a stirred solution of tert-butyl 3-(6-(methoxycarbonyl)pyridin-3-yl)-3,6-diazabicyclo[3.1.1]heptane-6-carboxylate (300 mg, 0.90 mmol) in MeOH (3 mL) was added MeNH₂ (33% in MeOH, 5 mL). The reaction was stirred at 25° C. for 18 h before it was concentrated under reduced pressure and purified by silica gel chromatography to afford tert-butyl 3-(6-(methylcarbamoyl)pyridin-3-yl)-3,6-diazabicyclo[3.1.1]heptane-6-carboxylate (270 mg, 90%). LC/MS m/z: 333.2 [M+H]⁺.

Step 3: Preparation of 5-(3,6-diazabicyclo[3.1.1]heptan-3-yl)-N-methylpicolinamide trifluoroacetate: To a stirred solution of tert-butyl 3-(6-(methylcarbamoyl)pyridin-3-yl)-3,6-diazabicyclo[3.1.1]heptane-6-carboxylate (270 mg, 0.81 mmol) in DCM (6 mL) was added TFA (2 mL). The reaction was stirred at 25° C. for 4 h. The reaction mixture was concentrated under reduced pressure to afford 5-(3,6-diazabicyclo[3.1.1]heptan-3-yl)-N-methylpicolinamide trifluoroacetate (300 mg, crude). LC/MS m/z: 233.1 [M+H]⁺.

Step 4: Preparation of 5-(6-((7-ethyl-6-oxo-5,6-dihydro-1,5-naphthyridin-3-yl)methyl)-3,6-diazabicyclo[3.1.1]heptan-3-yl)-N-methylpicolinamide formate: To a solution of 7-(chloromethyl)-3-ethyl-1,5-naphthyridin-2(1H)-one (170 mg, 0.76 mmol) in ACN (10 mL) were added 5-(3,6-diazabicyclo[3.1.1]heptan-3-yl)-N-methylpicolinamide trifluoroacetate (300 mg, crude), KI (252 mg, 1.52 mmol) and DIPEA (1.39 g, 10.8 mmol). The resulting solution was stirred at RT for 5 h before it was concentrated and purified by Prep-HPLC to afford 5-(6-((7-ethyl-6-oxo-5,6-dihydro-1,5-naphthyridin-3-yl)methyl)-3,6-diazabicyclo[3.1.1]heptan-3-yl)-N-methylpicolinamide formate (28 mg, 8%). LC/MS m/z: 419.1 [M+H]⁺; ¹H NMR (400 MHz, DMSO-d₆) δ 11.77 (s, 1H), 8.35 (dd, J=9.0, 4.0 Hz, 2H), 8.09-8.07 (m, 1H), 7.90-7.81 (m, 1H), 7.69 (s, 1H), 7.62 (s, 1H), 7.17 (dd, J=8.9, 2.9 Hz, 1H), 3.84-3.53 (m, 6H), 3.45-3.43 (m, 2H), 2.76 (d, J=4.8 Hz, 3H), 2.52-2.47 (m, 3H), 1.63-1.61 (m, 1H), 1.14 (q, J=7.5 Hz, 3H).

### Example B1. PARP1 Fluorescence Polarization Binding Assay

Compounds of the present disclosure were tested in a PARP1 fluorescence polarization binding assay at Pharmaron (Beijing, P.R. China). In this biochemical assay, binding of test compound to recombinant human PARP1 reduces the amount of binding of a fluorescent probe, and consequently reduces the detected level of fluorescence polarization. Test compound stock solutions were prepared in DMSO and then serially diluted into 10 concentrations by 3-fold dilution in a 384-well plate using a TECAN EVO200. 60 nL of each dilution was transferred to the plate using an Echo550 (Labcyte), and then 10 μL 40 nM GST-tagged PARP1 (BPS Bioscience, Cat #80501), diluted in assay buffer (50 mM Tris pH 8.0, 0.001% Triton X-100, 10 mM MgCl₂, 150 mM NaCl), was added to the plate. After a centrifugation at 1000 rpm for 1 min, the assay plate was incubated for 30 min at RT. 10 μL 6 nM PARPi-FL (TOCRIS, Cat #6461), diluted using assay buffer, was then added to the plate. The final concentrations of PARP1 and PARPi-FL were 20 nM and 3 nM, respectively, in a total volume of 20 μL. After a centrifugation at 1000 rpm for 1 min, the assay plate was incubated for 4 h at RT. Samples in each well were read using an Envision instrument (Perkin-Elmer; Ex=480 nm Em=FITC FP-P pol 535 nm & FITC FP-S pol 535 nm). Percent inhibition was calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. Binding IC₅₀ values were calculated using XLFit (equation 201: y=A+((B−A)/(1+((x/C)\hat{ }D))), where A=bottom, B=top, C=IC₅₀, and D=slope) with a floating top and bottom for curves. Binding IC₅₀ values are shown in Table A and are presented in ranges, in which “+++”<100 nM, 100 nM≤; “++”<3000 nM, and “+”≥3000 nM.

TABLE A

| Example No. | PARP1 IC₅₀ | PARP2 IC₅₀ |
|---|---|---|
| 3 | +++ | NA |
| 6 | +++ | + |
| 7 | ++ | + |
| 8 | ++ | NA |
| 9 | +++ | + |
| 29 | ++ | + |
| 139 | ++ | NA |
| 181 | + | NA |

### Example B2. PARP2 Fluorescence Polarization Binding Assay

Compounds of the present disclosure were tested in a PARP2 fluorescence polarization binding assay at Pharmaron (Beijing, P.R. China). In this biochemical assay, binding of test compound to recombinant human PARP2 reduces the amount of binding of a fluorescent probe, and consequently reduces the detected level of fluorescence polarization. Test compound stock solutions were prepared in DMSO and then serially diluted into 10 concentrations by 3-fold dilution in a 384-well plate using a TECAN EVO200. 60 nL of each dilution was transferred to the plate using an Echo550 (Labcyte), and then 10 μL 40 nM GST-tagged PARP2 (BPS Bioscience, Cat #80502), diluted in assay buffer (50 mM Tris pH 8.0, 0.001% Triton X-100, 10 mM MgCl₂, 150 mM NaCl), was added to the plate. After a centrifugation at 1000 rpm for 1 min, the assay plate was incubated for 30 min at RT. 10 μL 6 nM PARPi-FL (TOCRIS, Cat #6461), diluted using assay buffer, was then added to the plate. The final concentrations of PARP2 and PARPi-FL were 20 nM and 3 nM, respectively, in a total volume of 20 μL. After a centrifugation at 1000 rpm for 1 min, the assay plate was incubated for 4 h at RT. Samples in each well were read using an Envision instrument (Perkin-Elmer; Ex=480 nm Em=FITC FP-P pol 535 nm & FITC FP-S pol 535 nm). Percent inhibition was calculated from mP values using Inhibition (%)=[1−(mP_{c}−mP_{L})/(mP_{H}−mP_{L})]×100%, where mP_{c}, mP_{L}, and mP_{H} are the mP values of test compound, Low controls, and High controls, respectively. Binding IC₅₀ values were calculated using XLFit (equation 201: y=A+((B−A)/(1+((x/C)\hat{ }D))), where A=bottom, B=top, C=IC₅₀, and D=slope) with a floating top and bottom for curves. Binding IC₅₀ values are shown in Table A and are presented in ranges, in which “+++”<100 nM, 100 nM≤“++”<3000 nM, and “+”≥3000 nM.

### Example B3. Cell Viability Assay

Compounds of the present disclosure were tested for their effects on the viability of cancer cells. DLD-1 wild-type and DLD-1 BRCA2(−/−) colorectal adenocarcinoma cells, an isogenic pair of cell lines differing in the presence and absence, respectively, of both BRCA2 alleles, were used. The cells were harvested during the logarithmic growth period, counted, and seeded at 50 cells/well for DLD-1 wild-type and 200 cells/well for DLD-1 BRCA2(−/−) in a 384-well cell culture plate. After seeding, cells were incubated at 37° C., 5% CO₂ overnight. Cells were treated with serially diluted test compounds at 10 concentrations from 1.5 nM-30 μM) for generation of dose response curves. The plate was further incubated for another 7 days in a humidified incubator at 37° C. and 5% CO₂. Cell viability was assessed by luminescence measurement after addition of Cell Titer-Glo reagent (Promega, Madison, Wis.) according to the manufacturer's instructions. Cell viability IC₅₀ values were calculated using XLFit, equation 201: y=A+((B−A)/(1+((x/C)\hat{ }D))), where A=bottom, B=top, C=IC₅₀, and D=slope. IC₅₀ values are shown in Table B and are presented in ranges, in which “+++”<300 nM, 300 nM≤“++”<3000 nM, and “+”≥3000 nM. Effects of the test compounds on the viability of other cell lines such as MDA-MB-436, MDA-MB-231, SUM149PT, HCC1395, and UWB1.289 are determined in an analogous method.

TABLE B

| | DLD-1 | DLD-1 wild- |
|---|---|---|
| Example No. | BRCA2(−/−) IC₅₀ | type IC₅₀ |
| 3 | ++ | + |
| 6 | + | + |
| 7 | ++ | + |
| 8 | + | + |
| 9 | + | + |
| 29 | + | + |
| 139 | + | + |
| 181 | + | + |

### Numbered Embodiments

1. A compound of Formula (I):

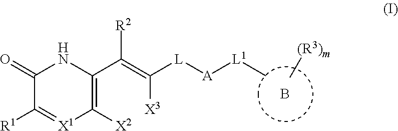

wherein:
A is an optionally substituted C₂₋₉ heterocyclyl comprising at least one nitrogen, or an optionally substituted C₃₋₈ cycloalkylene;
B is a C₆₋₁₀ aryl optionally substituted with one or more R³, or a C₂₋₉ heteroaryl optionally substituted with one or more R³
L is a bond, —CR⁴R⁵—, —C(O)—, —O—, —S—, —S(O)—, —S(O)₂—, —CR⁴R⁵O—, —OCR⁴R⁵—, —NR⁴—, —C(R⁴R⁵)NR^{4′}—, —C(O)NR^{4′}— or —NR^{4′}C(R⁴R⁵)—;
wherein R⁴, R^{4′} and R⁵ are independently —H, C₁₋₆ alkyl, or C₃₋₆ cycloalkyl, or R⁴ and R⁵ combine with the atom or atoms to which they are attached to form a C₂₋₉ heterocyclyl or C₃₋₈ cycloalkyl;
L¹ is a bond, —C(O)—, —NH—, —CH₂NH—, —O—, —CH₂O—, or —CH₂—;
X¹ is N or CR¹;
R¹ is —H, C₁₋₆ alkyl, C₃₋₆ cycloalkyl, C₁₋₆ haloalkyl, or C₁₋₆ alkoxy;
X² and X³ are each independently N or CR² provided that both X² and X³ are not both N;
R² is —H, halogen, C₁₋₆ alkyl, C₃₋₆ cycloalkyl, C₁₋₆ haloalkyl, or C₁₋₆ alkoxy;
R³ is halogen, C₁₋₆ alkyl, C₁₋₆ haloalkyl, C₁₋₆ alkoxy, —C(O)NR⁴R⁵, or —NR⁴C(O)R⁵; and
m is 1, 2, 3, or 4;
with the proviso that when (i) X¹ is N, X² is CH, and X³ is CH, or (ii) when X¹ is CH, X² is N, and X³ is CH, then A is not unsubstituted 1,4-piperazine; and with the proviso that when (i) A is a bridged, bicyclic piperazine, then R³ is not chloro;
or a pharmaceutically acceptable salt thereof.

2. The compound of embodiment 1, wherein the compound is of Formula (II):

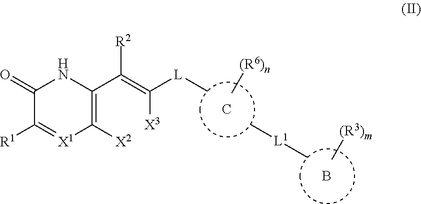

wherein C is a 4-8 membered heterocyclyl comprising at least one nitrogen atom;
R⁶ is a halogen, cyano, —OH, C₁₋₃ alkyl, or two R⁶ together form a carbonyl, C₃₋₆ spirocycl, C₄₋₆ spiroheterocyclyl, C₃₋₆ fused bicyclyl, or C₃₋₆ fused heterocyclyl;
L¹ is bond, —C(O)—, —O—, —CH₂O—, —NR⁷—, or —CH₂NR⁷—;
wherein R⁷=H, C₁₋₃ alkyl, or cyclopropyl and;
n=0, 1, 2, 3, or 4;
or a pharmaceutically acceptable salt thereof.

3. The compound of embodiment 2, wherein C is a spiro heterocyclyl, a fused heterocyclyl, or a bridged heterocyclyl.

4. The compound of embodiment 2, wherein C is a monocyclic heterocyclyl.

5. The compound of any one of embodiments 2 to 4, wherein the compound is of Formula (II-a):

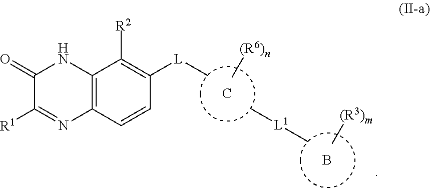

6. The compound of any one of embodiments 2 to 4, wherein the compound is of Formula (II-b):

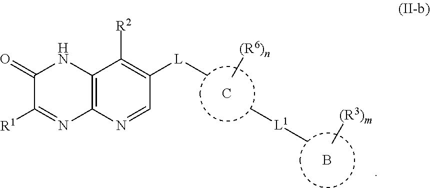

7. The compound of any one of embodiments 2 to 4, wherein the compound is of Formula (II-c):

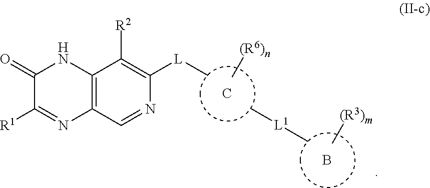

8. The compound of any one of embodiments 2 to 4, wherein the compound is of Formula (II-d):

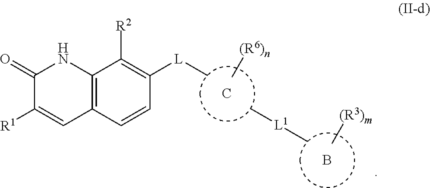

9. The compound of any one of embodiments 2 to 4, wherein the compound is of Formula (II-e):

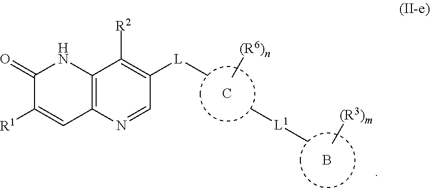

10. The compound of any one of embodiments 2 to 4, wherein the compound is of Formula (II-f):

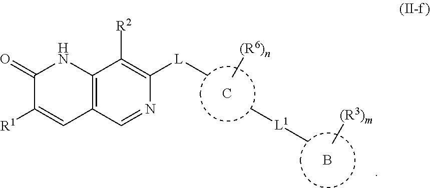

11. The compound of embodiment 1, wherein the compound is of Formula (III):

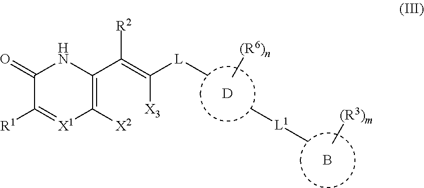

wherein D is a 3-8 membered carbocyclyl or bridged carbobicyclyl; and
R⁷ is H, or C₁₋₆ alkyl;
or a pharmaceutically acceptable salt thereof.

12. The compound of embodiment 11, wherein the compound is of Formula (III-a):

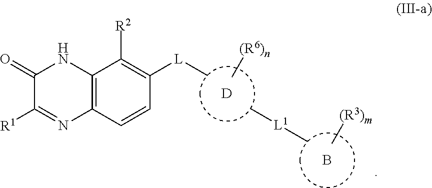

13. The compound of embodiment 11, wherein the compound is of Formula (III-b):

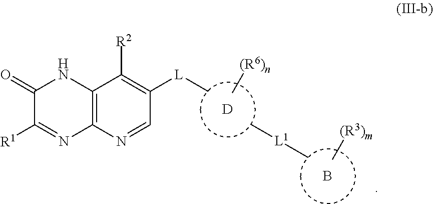

14. The compound of embodiment 11, wherein the compound is of Formula (III-c):

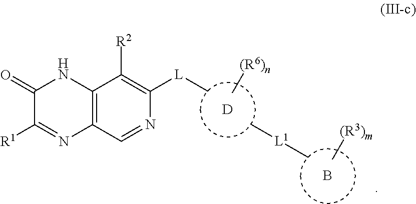

15. The compound of embodiment 11, wherein the compound is of Formula (III-d):

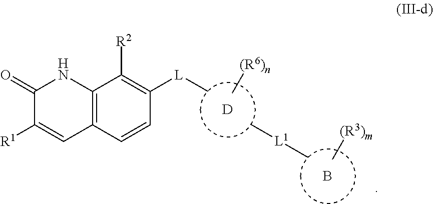

16. The compound of embodiment 11, wherein the compound is of Formula (III-e):

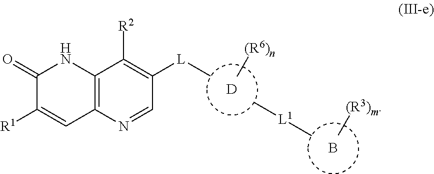

17. The compound of embodiment 11, wherein the compound is of Formula (III-f):

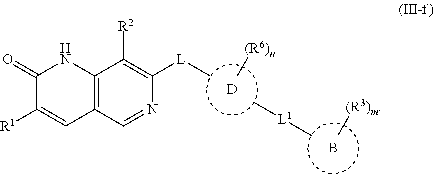

18. The compound of any one of embodiments 11 to 17, wherein L is —CR⁴R⁵—NR^{4′}— or —C(O)NR^{4′}—.

19. The compound of any one of embodiments 1 to 4 or 9, wherein the compound is of Formula (IV):

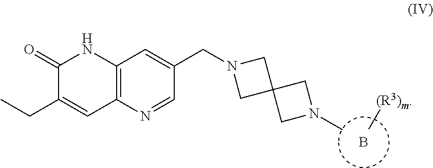

20. The compound of any one of embodiments 1 to 4 or 11, wherein:
R¹ is ethyl, X¹ is CH, X² is N, X³ is CH, and R² is H;
R¹ is methyl, X¹ is N, X² is CH, X³ is CH, and R² is fluoro;
R¹ is ethyl, X¹ is N, X² is N, X³ is CH, and R² is H;
R¹ is ethyl, X¹ is N, X² is CH, X³ is N, and R² is H;
R¹ is ethyl, X¹ is CH, X² is CH, X³ is CH, and R² is H;
R¹ is ethyl, X¹ is CH, X² is CH, X³ is N, and R² is H;
R¹ is methyl, X¹ is N, X² is CH, X³ is CH, and R² is H;
R¹ is trifluoromethyl, X¹ is N, X² is CH, X³ is CH, and R² is H; or
R¹ is 1,1,1-trifluoroethyl, X¹ is N, X² is CH, X³ is CH, and R² is H.

21. The compound of any one of embodiments 1 to 20, wherein B is a C₂₋₉ heteroaryl optionally substituted with one or more R³.

22. The compound of embodiment 21, wherein B is selected from

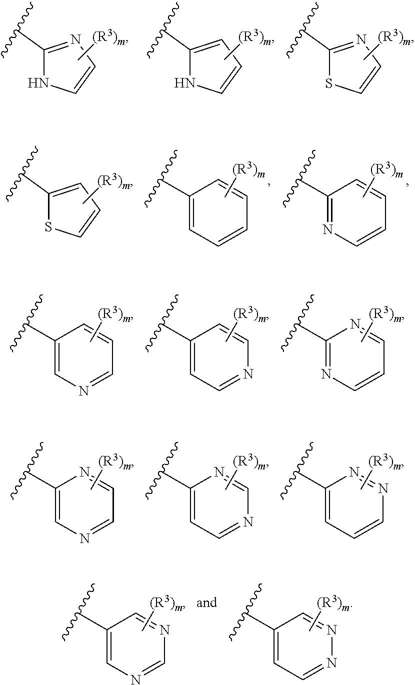

23. The compound of embodiment 22, wherein B is

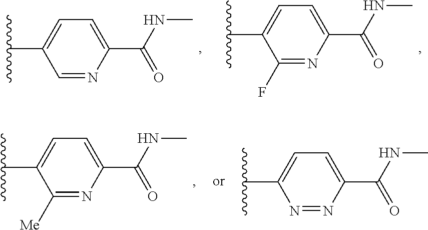

wherein [symbol] denotes points of attachment.

24. The compound of any one of embodiments 1 to 20, wherein B is a C₆₋₁₀ aryl optionally substituted with one or more R³.

25. The compound of embodiment 24, wherein B is

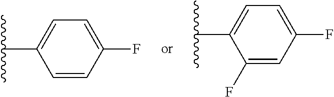

wherein [symbol] denotes points of attachment.

26. The compound of any one of embodiments 2 to 10 or 20 to 25, wherein C is

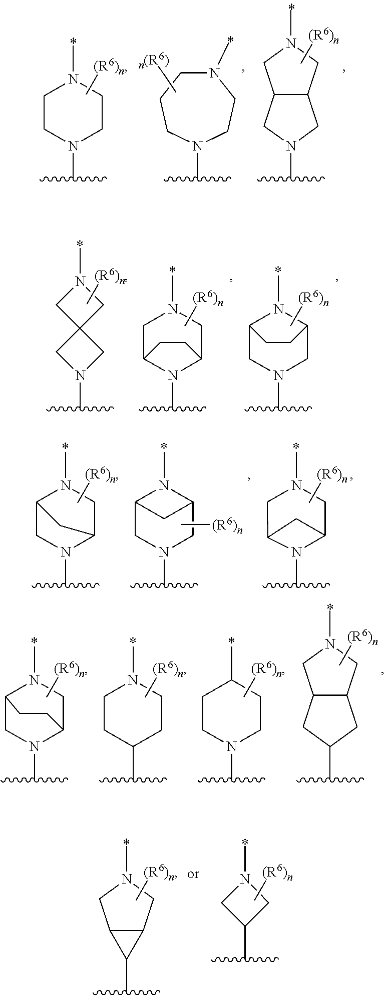

wherein * and [symbol] denote points of attachment.

27. The compound of any one of embodiments 2 to 10 or 20 to 26, wherein C is

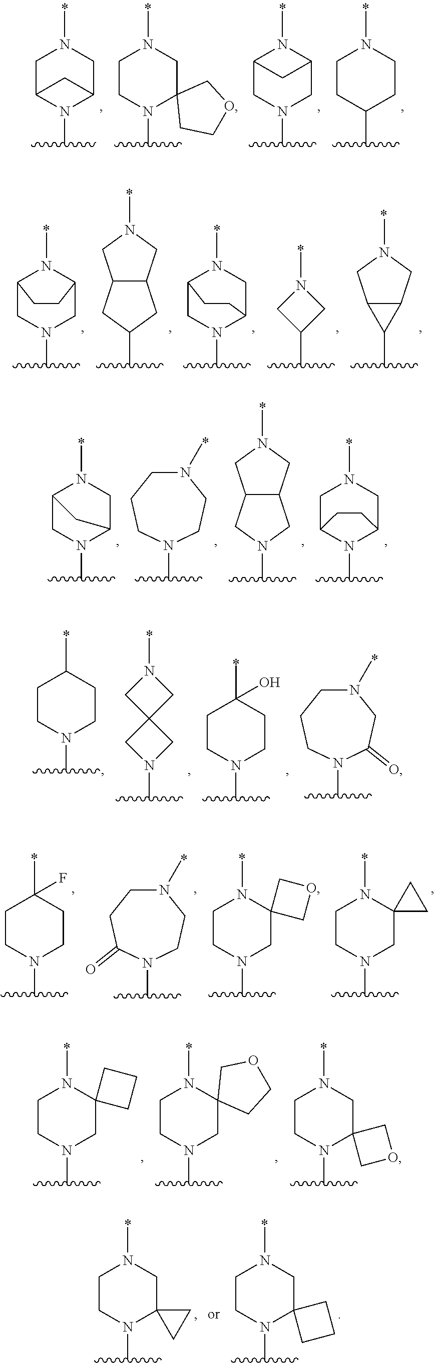

28. The compound of any one of embodiments 11 to 17 or 20 to 25, wherein D is

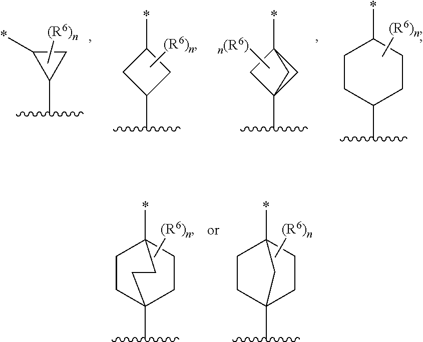

wherein * and [symbol] denote points of attachment.

29. The compound of any one of embodiments 11 to 17, 20 to 25, or 28, wherein D is

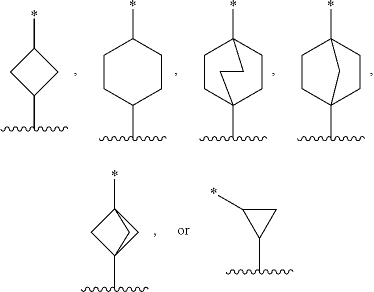

wherein * and [symbol] denote points of attachment.

30. The compound of any one of embodiments 1 to 17 or 20 to 29, wherein L is

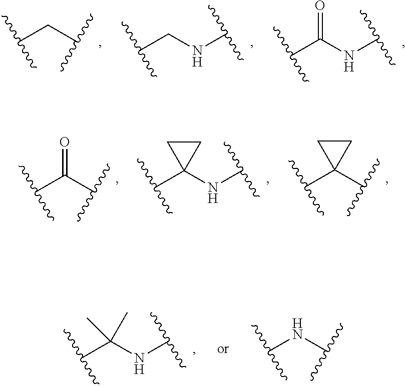

wherein [symbol] denotes points of attachment.

31. The compound of any one of embodiments 1 to 17 or 20 to 29, wherein L¹ is a bond,

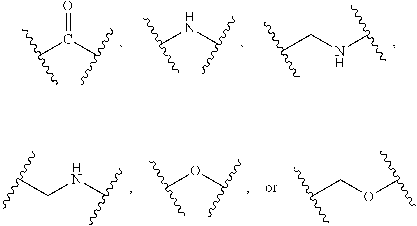

wherein [symbol] denotes points of attachment.

32. A compound having the structure of any one of the compounds in Table 1, or pharmaceutically acceptable salt thereof.

33. A pharmaceutical composition comprising the compound of any one of embodiments 1 to 32.

34. A method of treating a HR-deficient cancer, a BRCA1- or BRCA2-mutant cancer, or a cancer with a BRCAness phenotype in a subject, by administering an effective amount of the compound of any one of embodiments 1 to 33, or the pharmaceutical composition of embodiment 34, to a subject in need thereof.

35. The method of embodiment 34, wherein the cancer with a BRCAness phenotype is selected from the group consisting of breast cancer, ovarian cancer, prostate cancer, and pancreatic cancer.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 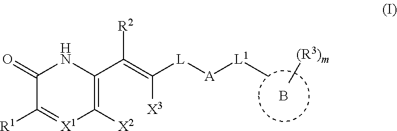
wherein:
A is an optionally substituted C₂₋₉ heterocyclyl comprising at least one nitrogen, or an optionally substituted C₃₋₈ cycloalkylene;
B is a C₆₋₁₀ aryl optionally substituted with one or more R³, or a C₂₋₉ heteroaryl optionally substituted with one or more R³;
L is a bond, —CR⁴R⁵—, —C(O)—, —O—, —S—, —S(O)—, —S(O)₂—, —CR⁴R⁵O—, —OCR⁴R⁵—, —NR⁴—, —C(R⁴R⁵)NR^{4′}—, —C(O)NR^{4′}— or —NR^{4′}C(R⁴R⁵)—;
wherein R⁴, R^{4′} and R⁵ are independently —H, C₁₋₆ alkyl, or C₃₋₆ cycloalkyl, or R⁴ and R⁵ combine with the atom or atoms to which they are attached to form a C₂₋₉ heterocyclyl or C₃₋₈ cycloalkyl;
L¹ is a bond, —C(O)—, —NH—, —CH₂NH—, —O—, —CH₂O—, or —CH₂—;
X¹ is N or CR¹;
R¹ is —H, C₁₋₆ alkyl, C₃₋₆ cycloalkyl, C₁₋₆ haloalkyl, or C₁₋₆ alkoxy;
X² and X³ are each independently N or CR² provided that both X² and X³ are not both N;
R² is —H, halogen, C₁₋₆ alkyl, C₃₋₆ cycloalkyl, C₁₋₆ haloalkyl, or C₁₋₆ alkoxy;
R³ is halogen, C₁₋₆ alkyl, C₁₋₆ haloalkyl, C₁₋₆ alkoxy, —C(O)NR⁴R⁵, or —NR⁴C(O)R⁵; and
m is 1, 2, 3, or 4;
with the proviso that when (i) X¹ is N, X² is CH, and X³ is CH, or (ii) when X¹ is CH, X² is N, and X³ is CH, then A is not unsubstituted 1,4-piperazine; and with the proviso that when (i) A is a bridged, bicyclic piperazine, then R³ is not chloro;
or a pharmaceutically acceptable salt thereof.

2. The compound of claim 1, wherein the compound is of Formula (II): 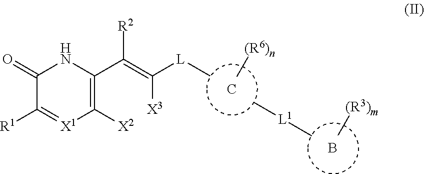
wherein C is a 4-8 membered heterocyclyl comprising at least one nitrogen atom;
R⁶ is a halogen, cyano, —OH, C₁₋₃ alkyl, or two R⁶ together form a carbonyl, C₃₋₆ spirocycl, C₄-6 spiroheterocyclyl, C₃₋₆ fused bicyclyl, or C₃₋₆ fused heterocyclyl;
L¹ is bond, —C(O)—, —O—, —CH₂O—, —NR⁷—, or —CH₂NR⁷—;
wherein R⁷=H, C₁₋₃ alkyl, or cyclopropyl and;
n is 0, 1, 2, 3, or 4;
or a pharmaceutically acceptable salt thereof.

3. The compound of claim 2, wherein C is a spiro heterocyclyl, a fused heterocyclyl, or a bridged heterocyclyl.

4. The compound of claim 2, wherein C is a monocyclic heterocyclyl.

5. The compound of claim 2, wherein the compound is of Formula (II-a):

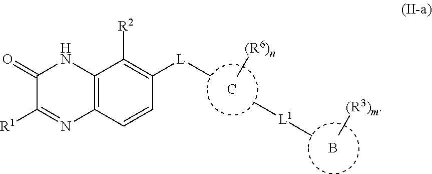

6. The compound of claim 2, wherein the compound is of Formula (II-b):

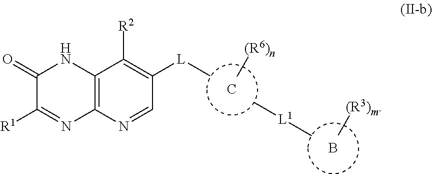

7. The compound of claim 2, wherein the compound is of Formula (II-c):

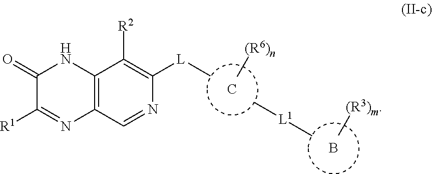

8. The compound of claim 2, wherein the compound is of Formula (II-d):

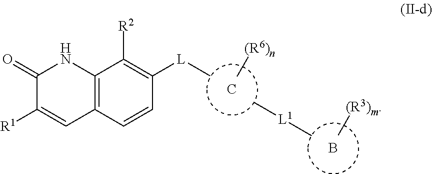

9. The compound of claim 2, wherein the compound is of Formula (II-e):

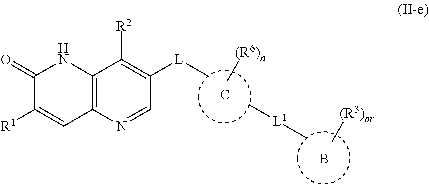

10. The compound of claim 2, wherein the compound is of Formula (II-f):

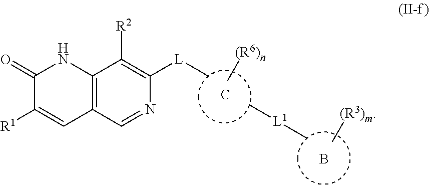

11. The compound of claim 1, wherein the compound is of Formula (III): 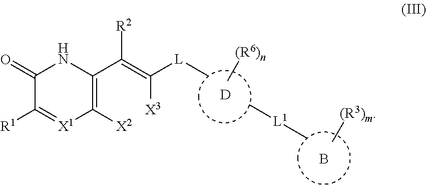
Wherein D is a 3-8 membered carbocyclyl or bridged carbobicyclyl; and
R⁷ is H, or C₁₋₆ alkyl;
or a pharmaceutically acceptable salt thereof.

12. The compound of claim 11, wherein the compound is of Formula (III-a):

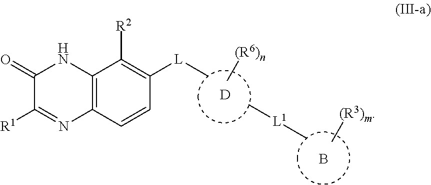

13. The compound of claim 11, wherein the compound is of Formula (III-b):

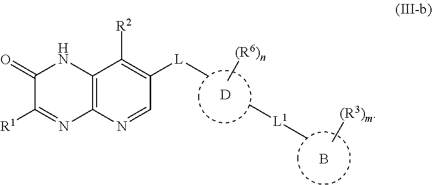

14. The compound of claim 11, wherein the compound is of Formula (III-c):

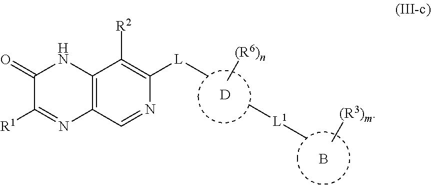

15. The compound of claim 11, wherein the compound is of Formula (III-d):

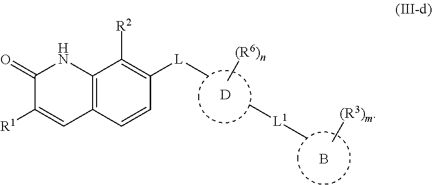

16. The compound of claim 11, wherein the compound is of Formula (III-e):

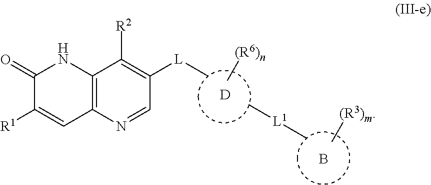

17. The compound of claim 11, wherein the compound is of Formula (III-f):

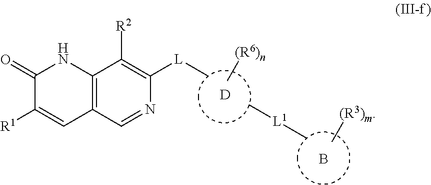

18. The compound of claim 11, wherein L is —CR⁴R⁵—NR^{4′}— or —C(O)NR^{4′}—.

19. The compound of claim 1, wherein:
R¹ is ethyl, X¹ is CH, X² is N, X³ is CH, and R² is H;
R¹ is methyl, X¹ is N, X² is CH, X³ is CH, and R² is fluoro;
R¹ is ethyl, X¹ is N, X² is N, X³ is CH, and R² is H;
R¹ is ethyl, X¹ is N, X² is CH, X³ is N, and R² is H;
R¹ is ethyl, X¹ is CH, X² is CH, X³ is CH, and R² is H;
R¹ is ethyl, X¹ is CH, X² is CH, X³ is N, and R² is H;
R¹ is methyl, X¹ is N, X² is CH, X³ is CH, and R² is H;
R¹ is trifluoromethyl, X¹ is N, X² is CH, X³ is CH, and R² is H; or
R¹ is 1,1,1-trifluoroethyl, X¹ is N, X² is CH, X³ is CH, and R² is H.

20. The compound of claim 1, wherein B is a C₂₋₉ heteroaryl optionally substituted with one or more R³.

21. The compound of claim 20, wherein B is selected from

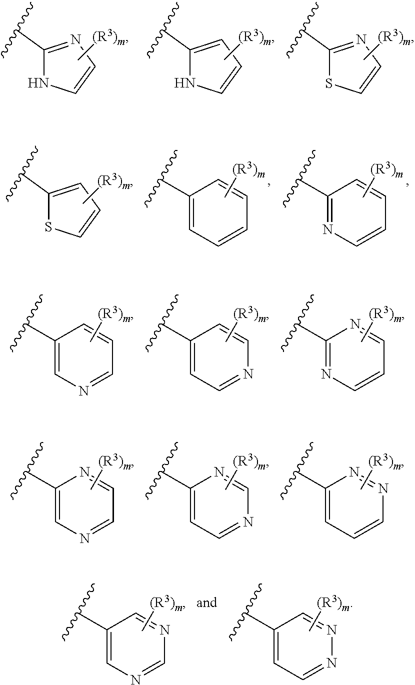

22. The compound of claim 21, wherein B is 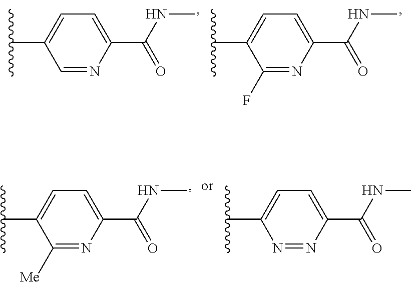
wherein [symbol] denotes points of attachment.

23. The compound of claim 1, wherein B is a C₆₋₁₀ aryl optionally substituted with one or more R³.

24. The compound of claim 23, wherein B is 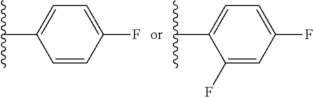
wherein [symbol] denotes points of attachment.

25. The compound of claim 2, wherein C is 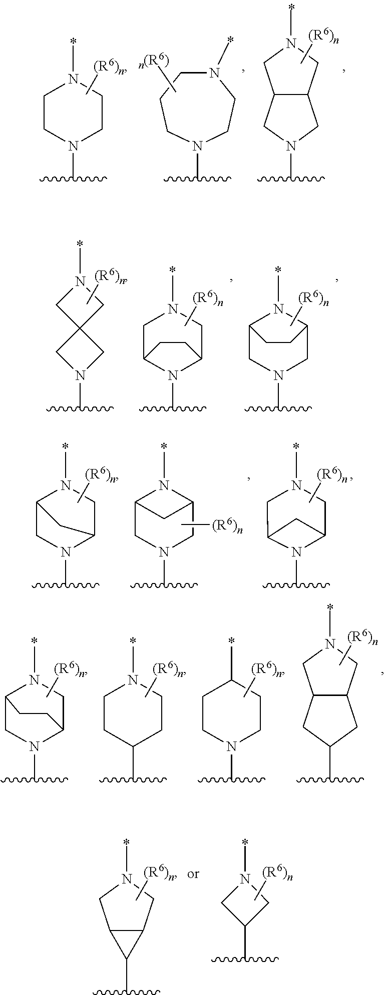
wherein * and [symbol] denote points of attachment.

26. The compound of claim 2, wherein C is

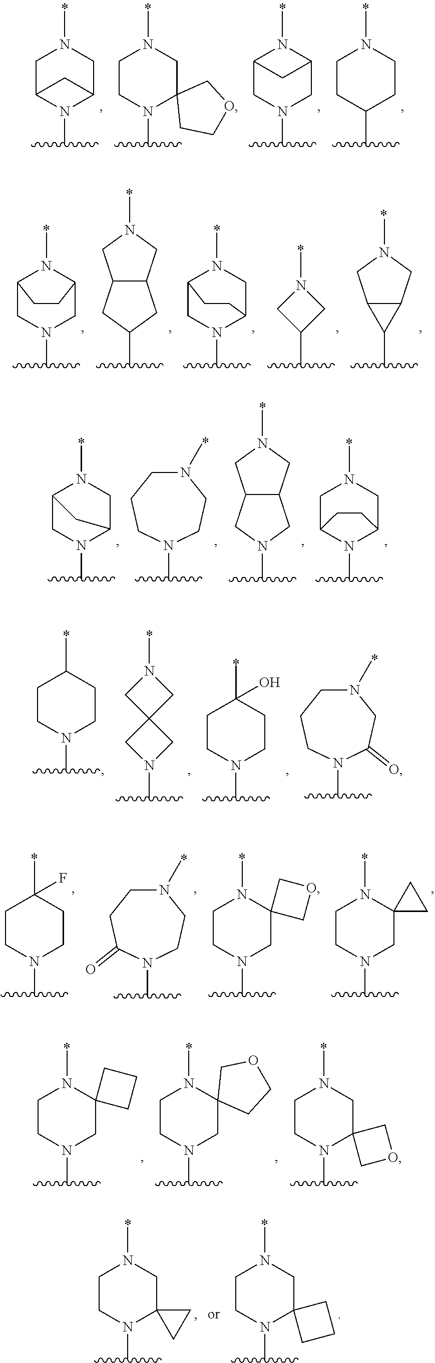

27. The compound of claim 11, wherein D is 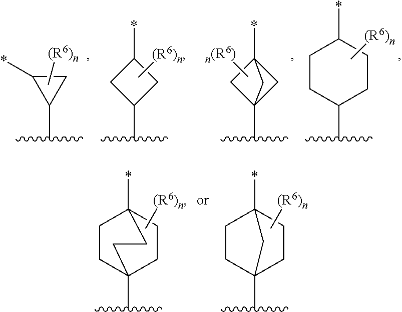
wherein * and [symbol] denote points of attachment.

28. The compound of claim 11, wherein D is 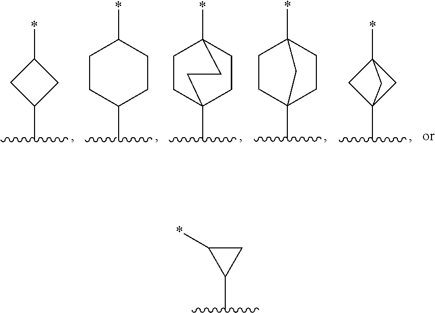
or wherein * and [symbol] denote points of attachment.

29. The compound claim 1, wherein L is 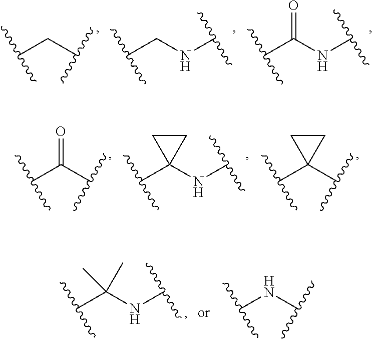
wherein [symbol] denotes points of attachment.

30. The compound of claim 1, wherein L¹ is a bond 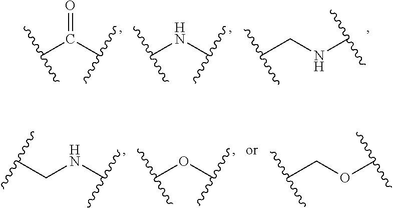
wherein [symbol] denotes points of attachment.

31. A compound having the structure of any one of the compounds in Table 1, or pharmaceutically acceptable salt thereof.

32. A pharmaceutical composition comprising the compound of claim 1, or a pharmaceutically acceptable salt thereof, and a pharmaceutically acceptable excipient.

33. A method of treating a cancer comprising administering an effective amount of the compound of claim 1, or the pharmaceutical composition of claim 32, to a subject in need thereof.

34. The method of claim 33, wherein the cancer is a breast cancer, ovarian cancer, prostate cancer, or pancreatic cancer.

35. The method of claim 33, wherein the cancer is an HR-deficient cancer, a BRCA1- or BRCA2-mutant cancer, or a cancer with a BRCAness phenotype.
